(19) United States
(12) Patent Application Publication
(10) Pub. No.: US 2013/0338176 A1
(43) Pub. Date: Dec. 19, 2013

(54) METHODS OF PREVENTING AND TREATING HYPERLIPIDEMIA OR ATHEROSCLEROSIS

(71) Applicants: Gerard Karsenty, New York, NY (US); Grzegorz Sumara, Wuerzburg (DE)

(72) Inventors: Gerard Karsenty, New York, NY (US); Grzegorz Sumara, Wuerzburg (DE)

(73) Assignee: The Trustees of Columbia University in the City of New York, New York, NY (US)

(21) Appl. No.: 13/882,394

(22) PCT Filed: Oct. 28, 2011
(86) PCT No.: PCT/US11/58396

§ 371 Date: Sep. 6, 2013

Related U.S. Application Data
(60) Provisional application No. 61/408,265, filed on Oct. 29, 2010.

(51) Int. Cl. A61K 31/505; A61K 45/06

(52) U.S. Cl. CPC A61K 31/505; A61K 45/06
USPC 514/272

(57) ABSTRACT

The present invention provides methods and therapeutic agents for lowering serum or plasma serotonin levels in a patient in order to prevent or treat hyperlipidemia or atherosclerosis. In preferred embodiments, the patient is known to have, or to be at risk for, hyperlipidemia or atherosclerosis and the agents are TPH1 inhibitors.

### CROSS-REFERENCE TO RELATED APPLICATIONS

This application claims priority from U.S. Provisional Patent Application Ser. No. 61/408,265, filed Oct. 29, 2010, the disclosures of which are incorporated herein by reference in their entirety.

### STATEMENT OF GOVERNMENT INTEREST

The invention was made with Government support under NIH 2 ROI DK 067936. The Government has certain rights in the invention.

### FIELD OF THE INVENTION

The invention is in the field of prevention and therapy of hyperlipidemia or atherosclerosis and provides methods for lowering plasma levels of cholesterol, triglycerides, glycerol, and free fatty acids in mammals, particularly in humans.

### BACKGROUND OF THE INVENTION

Atherosclerosis and its complications are one of the most common causes of death in Western societies. Atherosclerosis is a multistep disease initiated by the formation of fatty streak lesions in blood vessels, followed by lesion progression and the formation of plaques, plaque rupture, and thrombosis (Glass & Witztum, 2001, Cell 104:503-516; Libby, 2002, Nature 420:868-874).

In normal circumstances the endothelial monolayer in contact with flowing blood resists firm adhesion of monocytes. However, upon exposure to pro-inflammatory factors there is a steady increase in the expression of various leukocyte adhesion molecules in endothelial cells, which enables monocytes to adhere to the endothelial cell membranes (Libby, 2002, Nature 420:868-874). Adhesion is followed by transmigration of monocytes through the endothelial layer into the intima. This process is governed by chemotactic factors produced in the subendothelial layer and in the intima. Once they have migrated, monocytes become tissue-resident macrophages, which in turn develop into lipid-loaded foam cells upon exposure to modified lipoproteins (Osterud and Bjorklid, 2003, Physiol. Rev. 83:1069-1112). Migration of the vascular smooth muscle cells (VSMC) into intima is the hallmark of the transition of fatty streak into the more complex plaques. VSMC proliferate in the intima, take up modified lipoproteins, and eventually become foam cells. Moreover, they secrete substantial amounts of extracellular matrix proteins, promoting development of a fibrous cap (Glass and Witztum, 2001, Cell 104:503-516). Advanced atherosclerotic lesions cause narrowing of the blood vessels, which results in ischemic changes in peripheral organs. In addition, at this stage, atherosclerotic plaques after rupture expose tissue factors and plaque lipids to blood and initiate the process of blood coagulation and the formation of thrombus cap, often resulting in myocardial infarction (Glass and Witztum, 2001, Cell 104:503-516).

High blood cholesterol and triglyceride levels are the main triggers for development of atherosclerotic plaques. In the blood, cholesterol and triglycerides are transported in complexes with certain proteins known as lipoproteins. Three main classes of lipoproteins can be distinguished; very low density lipoproteins (VLDL), low density lipoproteins (LDL), and high density lipoproteins (HDL). The distribution of cholesterol over different classes of lipoproteins is crucial for its pro-atherogenic effects. In general, high blood levels of VLDL and LDL predispose towards the development of atherosclerosis. In contrast, high levels of HDL protect against atherogenesis (Rader and Daugherty, 2008, Nature 451:904-913).

Serotonin (5-hydroxytryptamine, 5-HT) is a biogenic amine that functions both as a neurotransmitter in the mammalian central nervous system and as a hormone in the periphery, where most of it is produced (Gershon et al., 1990, Neuropsychopharmacology, 3:385-395). Serotonin is generated through an enzymatic cascade in which L-tryptophan is converted into L-5-hydroxytryptophan by an enzyme called tryptophan hydroxylase (TPH). This intermediate product is then converted to serotonin by an aromatic L-amino acid decarboxylase. There are two TPH encoding genes, TPH1 and TPH2, which are 71% identical in amino acid sequence and about 90% similar in their catalytic domains. While TPH1 controls serotonin synthesis in the periphery, TPH2 is responsible for serotonin synthesis in the brain (Walther et al., 2003, Science 299:76). Given that serotonin cannot cross the blood-brain barrier, these two genes are therefore solely responsible for regulating the level of this molecule in the periphery and in the brain, respectively.

TPH1 is expressed almost exclusively in cells of the duodenum, and it is responsible for the synthesis of peripheral serotonin, which represents 95% of total serotonin (Gershon & Tack, 2007, Gastroenterology 132:397-414). TPH1 expression in any tissues other than duodenum is at least 100-1000 fold lower. Thus, TPH1 can be viewed as a duodenum-specific gene and peripheral serotonin production as a duodenum-specific process.

Besides its role as a neuromediator, and because of its abundance in the general circulation, serotonin has been implicated in a variety of developmental and physiological processes in peripheral tissues, including heart development, gastrointestinal movement, liver regeneration and mammary gland development (Lesurtel et al., 2006, Science, 312:104-107; Matsuda et al., 2004, Dev. Cell, 6:193-203; Nebigil et al., 2000, Proc. Natl. Acad. Sci. USA 97:9508-9513). To carry out its functions, serotonin can bind to at least 14 receptors, most of them being G-protein coupled receptors (GPCRs). One or several serotonin receptors are present in most cell types.

Mice genetically deficient for the TPH1 gene (“knockout mice”) have been reported. In one case, the mice reportedly expressed normal amounts of serotonin in classical serotonergic brain regions, but largely lacked serotonin in the periphery. In another, the knockout mice exhibited abnormal cardiac activity, which was attributed to a lack of peripheral serotonin (Cote et al., 2003, Proc. Natl. Acad. Sci. USA 100:13525-13530).

International Patent Application No. PCT/US2009/038817, published as WO 2009/123978, the disclosure of which is incorporated herein in its entirety, is directed to methods of diagnosing, preventing, and treating bone mass diseases using therapeutic agents for lowering or increasing serum serotonin levels. International Patent Application No. PCT/US2009/064383, published as WO 2010/056992, the disclosure of which is incorporated herein in its entirety, is also directed to methods of diagnosing, preventing, and treating bone mass diseases using therapeutic agents for lowering or increasing serum serotonin levels.

LP-533401 is an inhibitor of TPH1 having the following structure:

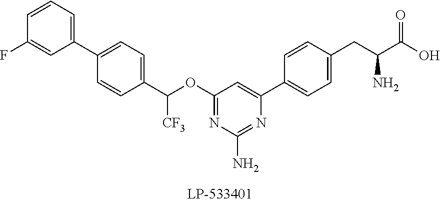 [SMILES: Nc1nc(OC(c2ccc(-c3cccc(F)c3)cc2)C(F)(F)F)cc(-c2ccc(C[C@H](N)C(=O)O)cc2)n1]

See, e.g., Liu et al., 2008, J. Pharmacol. Exp. Ther. 325:47-55.

Other inhibitors of TPH1 are disclosed in International Patent Publications WO 09/123978; WO 10/056992; WO 08/073933; WO 09/002964; WO 09/002970; WO 09/009561; WO 09/014972; WO 09/029499; WO 09/042733; WO 09/048864; WO 10/065333; and WO 07/089335. Other disclosures of TPH1 inhibitors appear in U.S. Pat. No. 7,553,840 and U.S. Patent Application Publications Nos. US 2007/0191370; US 2008/0153852; US 2009/0005381; US 2009/0005382; US 2009/0029993; US 2009/0054308; US 2009/0062540; US 2009/0088447; and US 2009/0099206.

### SUMMARY OF THE INVENTION

The present invention provides therapeutic agents that are inhibitors of tryptophan hydroxylase 1 (TPH1), the enzyme responsible for the first step of serotonin synthesis in enterochromaffin cells of the duodenum, for use in the treatment and/or prevention of hyperlipidemia or atherosclerosis. Also provided are pharmaceutical compositions comprising the therapeutic agents, for use in the treatment and/or prevention of hyperlipidemia or atherosclerosis.

It has been discovered that serum or plasma serotonin in mammals is involved in the control of blood levels of cholesterol, triglycerides, glycerol, and free fatty acids. High levels of cholesterol, triglycerides, glycerol, and free fatty acids have been linked to hyperlipidemia and atherosclerosis. Thus, in certain embodiments, the present invention provides a method of treating or preventing hyperlipidemia or atherosclerosis in a patient known or suspected to be in need of such treatment or prevention comprising administering to the patient known or suspected to be in need of such treatment or prevention a therapeutically effective amount of a TPH1 inhibitor.

In certain embodiments, the present invention provides a method of lowering plasma cholesterol, lowering plasma triglycerides, lowering plasma glycerol, or lowering plasma free fatty acids in a patient known or suspected to be in need of such lowering of plasma cholesterol, lowering of plasma triglycerides, lowering of plasma glycerol, or lowering of plasma free fatty acids comprising administering to the patient a therapeutically effective amount of a TPH1 inhibitor.

Other methods disclosed herein are directed to diagnosing a person at risk of developing hyperlipidemia or atherosclerosis by determining if the person's level of serum or plasma serotonin is abnormally high (about 25% or more) compared to normal individuals, taking into account the age, gender, or other factors that affect serum or plasma serotonin levels. Such a person at risk may be treated with therapeutic agents that decrease serum or plasma serotonin to prevent hyperlipidemia or atherosclerosis from developing, or to slow the development of hyperlipidemia or atherosclerosis. Those of skill in the art will understand that serum or plasma serotonin levels may vary among individuals depending on certain factors and will be able to take those factors into account to determine whether a person has abnormally high serum or plasma serotonin levels. One possible range which those skilled in the art may consider to be normal serum or plasma serotonin levels is 101-283 ng/ml (nanograms per milliliter).

Since elevated serum or plasma serotonin may not be the only cause of hyperlipidemia or atherosclerosis, methods other than those measuring serum or plasma serotonin levels may also be used to determine if a person is at risk of developing hyperlipidemia or atherosclerosis and should be treated with therapeutic agents that decrease serum or plasma serotonin.

The present invention provides a method of lowering serum or plasma serotonin levels in a patient known or suspected to be in need of lowering of serum or plasma serotonin levels in order to treat or prevent hyperlipidemia or atherosclerosis comprising administering a TPH1 inhibitor to the patient known or suspected to be in need of lowering of serum or plasma serotonin levels in order to treat or prevent hyperlipidemia or atherosclerosis.

The present invention also provides a method of treating or preventing hyperlipidemia or atherosclerosis in a patient known or suspected to be in need of such treatment or prevention comprising administering to the patient known or suspected to be in need of such treatment or prevention a therapeutically effective amount of a therapeutic agent that lowers the level of serum or plasma serotonin.

In preferred embodiments, the therapeutic agent is a TPH1 inhibitor that does not cross the blood brain barrier. In other embodiments, the therapeutic agent is a TPH1 inhibitor that does not significantly inhibit TPH2.

In one embodiment, the present invention provides a method of treating or preventing hyperlipidemia or atherosclerosis in a patient known or suspected to be in need of such treatment or prevention comprising administering to the patient known or suspected to be in need of such treatment or prevention a therapeutically effective amount of a TPH1 inhibitor.

In one embodiment, the present invention provides a method of lowering blood levels of cholesterol, triglycerides, glycerol, and/or free fatty acids in a patient known or suspected to be in need of such lowering comprising administering to the patient known or suspected to be in need of such lowering a therapeutically effective amount of a TPH1 inhibitor.

In certain embodiments, the TPH1 inhibitor is selected from the following or from pharmaceutically acceptable salts and/or solvates thereof:

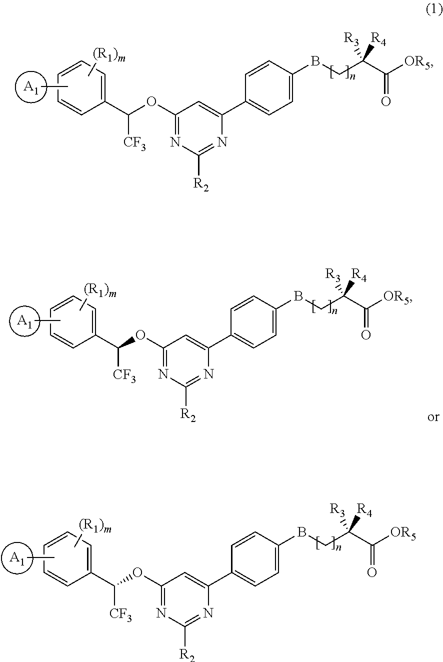

where A₁ is optionally substituted heterocycle or 3-fluorophenyl; B is O, N, or —CH₂—; each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is halogen, hydrogen, C(O)R_{A}, OR_{A}, NR_{B}R_{C}, S(O₂)R_{A}, or optionally substituted alkyl, alkyl-aryl or alkyl-heterocycle; either R₃ is NHR₆ and R₄ is hydrogen or, alternatively, R₃ and R₄ together form ═O; R₅ is hydrogen or optionally substituted alkyl, alkyl-aryl, alkyl-heterocycle, aryl, or heterocycle; R₆ is hydrogen, C(O)R_{A}, C(O)OR_{A}, or optionally substituted alkyl, alkyl-aryl, alkyl-heterocycle, aryl, or heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; m is 0-4; and n is 0 or 1.

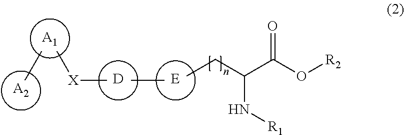

where each of A₁ and A₂ is independently a monocyclic optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; E is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; each R₃ is independently hydrogen, alkoxy, amino, cyano, halogen, hydroxyl, or optionally substituted alkyl; each R₄ is independently hydrogen, alkoxy, amino, cyano, halogen, hydroxyl, or optionally substituted alkyl or aryl; each R₅ is independently hydrogen or optionally substituted alkyl or aryl; and n is 0-3.

Compounds encompassed by the formula immediately above include those wherein A₁ and/or A₂ is optionally substituted cycloalkyl (e.g., 6-membered and 5-membered). In some, A₁ and/or A₂ is optionally substituted aryl (e.g., phenyl or naphthyl). In others, A₁ and/or A₂ is optionally substituted heterocycle (e.g., 6-membered and 5-membered). Examples of 6-membered heterocycles include pyridine, pyridazine, pyrimidine, pyrazine, and triazine. Examples of 5-membered heterocycles include pyrrole, imidazole, triazole, thiazole, thiophene, and furan. In some compounds, A₁ and/or A₂ is aromatic. In others, A₁ and/or A₂ is not aromatic.

Particular compounds include those wherein D is optionally substituted aryl (e.g., phenyl or naphthyl). In others, D is optionally substituted heterocycle (e.g., 6-membered and 5-membered). Examples of 6-membered heterocycles include pyridine, pyridazine, pyrimidine, pyrazine, and triazine. Examples of 5-membered heterocycles include pyrrole, imidazole, triazole, thiazole, thiophene, and furan. In some compounds, D is aromatic. In others, D is not aromatic. In some, D is an optionally substituted bicyclic moiety (e.g., indole, iso-indole, pyrrolo-pyridine, or napthylene).

Particular compounds include those wherein E is optionally substituted aryl (e.g., phenyl or naphthyl). In others, E is optionally substituted heterocycle (e.g., 6-membered and 5-membered). Examples of 6-membered heterocycles include pyridine, pyridazine, pyrimidine, pyrazine, and triazine. Examples of 5-membered heterocycles include pyrrole, imidazole, triazole, thiazole, thiophene, and furan. In some compounds, E is aromatic. In others, E is not aromatic. In some, E is an optionally substituted bicyclic moiety (e.g., indole, iso-indole, pyrrolo-pyridine, or napthylene).

Particular compounds include those wherein R₁ is hydrogen or optionally substituted alkyl.

In some compounds, R₂ is hydrogen or optionally substituted alkyl.

In some compounds, n is 1 or 2.

In some compounds, X is a bond or S. In others, X is —C(R₄)═, ═C(R₄)—, —C(R₃R₄)—, —C(R₄)═C(R₄)—, or —C≡C—, and, for example, R₄ is independently hydrogen or optionally substituted alkyl. In others, X is —O—, —C(R₃R₄)O—, or —OC(R₃R₄)—, and, for example, R₃ is hydrogen or optionally substituted alkyl, and R₄ is hydrogen or optionally substituted alkyl. In some, R₃ is hydrogen and R₄ is trifluoromethyl. In some compounds, X is —S(O₂)—, —S(O₂)N(R₅)—, —N(R₅)S(O₂)—, —C(R₃R₄)S(O₂)—, or —S(O₂)C(R₃R₄)—, and, for example, R₃ is hydrogen or optionally substituted alkyl, R₄ is hydrogen or optionally substituted alkyl, and R₅ is hydrogen or optionally substituted alkyl. In others, X is —N(R₅)—, —N(R₅)C(O)N(R₅)—, —C(R₃R₄)N(R₅)—, or —N(R₅)C(R₃R₄)—, and, for example, R₃ is hydrogen or optionally substituted alkyl, R₄ is hydrogen or optionally substituted alkyl, and each R₅ is independently hydrogen or optionally substituted alkyl.

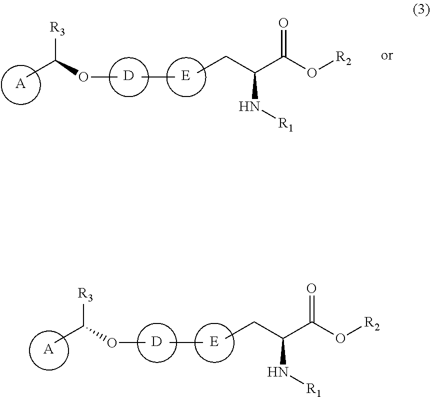

where A is optionally substituted cycloalkyl, aryl, or heterocycle; D is optionally substituted aryl or heterocycle; E is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; and R₃ is trifluoromethyl.

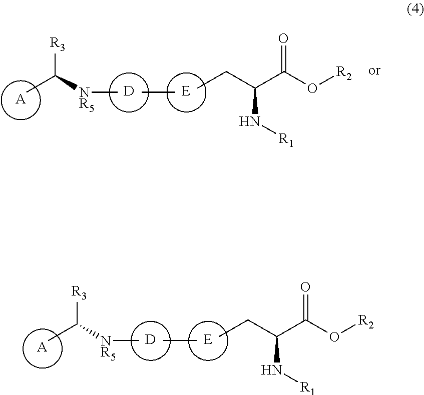

where A is optionally substituted cycloalkyl, aryl, or heterocycle; D is optionally substituted aryl or heterocycle; E is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen; and R₅ is hydrogen or optionally substituted alkyl or aryl.

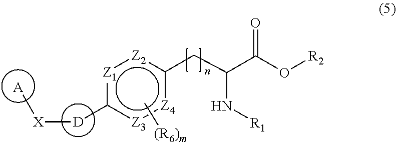

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; each of Z₁, Z₂, Z₃, and Z₄ is independently N or CR₆; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; each R₃ is independently hydrogen, alkoxy, amino, cyano, halogen, hydroxyl, or optionally substituted alkyl; each R₄ is independently hydrogen, alkoxy, amino, cyano, halogen, hydroxyl, or optionally substituted alkyl or aryl; each R₅ is independently hydrogen or optionally substituted alkyl or aryl; each R₆ is independently hydrogen, cyano, halogen, OR₇, NR₈R₉, amino, hydroxyl, or optionally substituted alkyl, alkyl-aryl or alkyl-heterocycle; each R₇ is independently hydrogen or optionally substituted alkyl, alkyl-aryl or alkyl-heterocycle; each R₈ is independently hydrogen or optionally substituted alkyl, alkyl-aryl or alkyl-heterocycle; each R₉ is independently hydrogen or optionally substituted alkyl, alkyl-aryl or alkyl-heterocycle; m is 1-4; and n is 1-3.

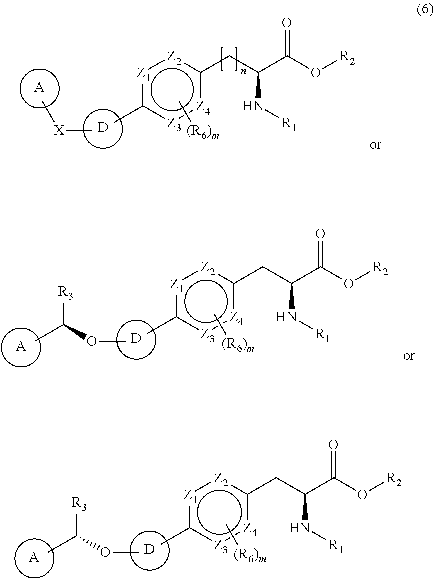

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; each of Z₁, Z₂, Z₃, and Z₄ is independently N or CR₆; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is trifluoromethyl; each R₄ is independently hydrogen, alkoxy, amino, cyano, halogen, hydroxyl, or optionally substituted alkyl or aryl; each R₅ is independently hydrogen or optionally substituted alkyl or aryl; each R₆ is independently hydrogen, cyano, halogen, OR₇, NR₈R₉, amino, hydroxyl, or optionally substituted alkyl, alkyl-aryl or alkyl-heterocycle; each R₇ is independently hydrogen or optionally substituted alkyl, alkyl-aryl or alkyl-heterocycle; each R₈ is independently hydrogen or optionally substituted alkyl, alkyl-aryl or alkyl-heterocycle; each R₉ is independently hydrogen or optionally substituted alkyl, alkyl-aryl or alkyl-heterocycle; m is 1-4; and n is 1-3.

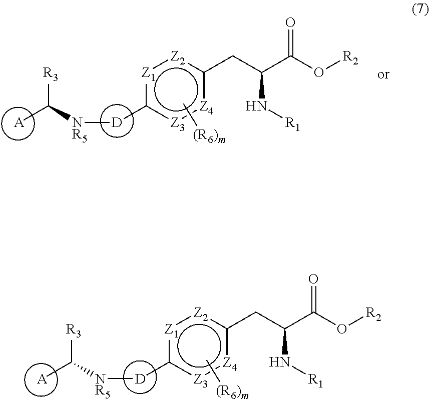

where A is optionally substituted cycloalkyl, aryl, or heterocycle; D is optionally substituted aryl or heterocycle; each of Z₁, Z₂, Z₃, and Z₄ is independently N or CR₆; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen; R₅ is hydrogen or optionally substituted alkyl or aryl; each R₆ is independently hydrogen, cyano, halogen, OR₇, NR₈R₉, amino, hydroxyl, or optionally substituted alkyl, alkyl-aryl or alkyl-heterocycle; each R₇ is independently hydrogen or optionally substituted alkyl, alkyl-aryl or alkyl-heterocycle; each R₈ is independently hydrogen or optionally substituted alkyl, alkyl-aryl or alkyl-heterocycle; each R₉ is independently hydrogen or optionally substituted alkyl, alkyl-aryl or alkyl-heterocycle; and m is 1-4.

Some compounds are such that all of Z₁, Z₂, Z₃, and Z₄ are N. In others, only three of Z₁, Z₂, Z₃, and Z₄ are N. In others, only two of Z₁, Z₂, Z₃, and Z₄ are N. In others, only one of Z₁, Z₂, Z₃, and Z₄ is N. In others, none of Z₁, Z₂, Z₃, and Z₄ are N.

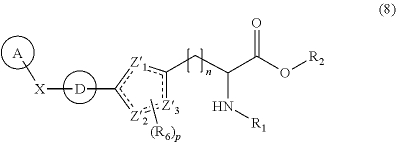

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; each of Z′₁, Z′₂, and Z′₃, is independently N, NH, S, O or CR₆; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; each R₃ is independently hydrogen, alkoxy, amino, cyano, halogen, hydroxyl, or optionally substituted alkyl; each R₄ is independently hydrogen, alkoxy, amino, cyano, halogen, hydroxyl, or optionally substituted alkyl or aryl; each R₅ is independently hydrogen or optionally substituted alkyl or aryl; each R₆ is independently amino, cyano, halogen, hydrogen, OR₇, SR₇, NR₈R₉, or optionally substituted alkyl, alkyl-aryl or alkyl-heterocycle; each R₇ is independently hydrogen or optionally substituted alkyl, alkyl-aryl or alkyl-heterocycle; each R₈ is independently hydrogen or optionally substituted alkyl, alkyl-aryl or alkyl-heterocycle; each R₉ is independently hydrogen or optionally substituted alkyl, alkyl-aryl or alkyl-heterocycle; n is 1-3; and p is 1-3.

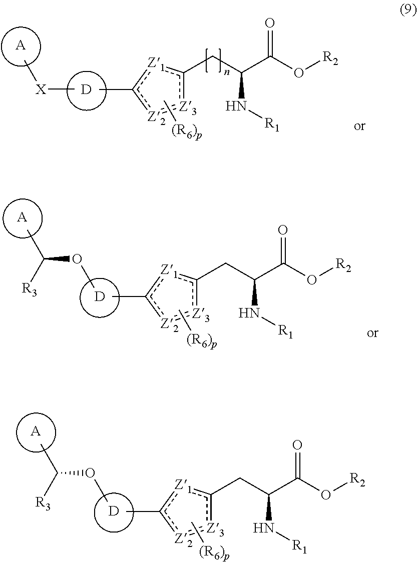

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; each of Z′₁, Z′₂, and Z′₃, is independently N, NH, S, O or CR₆; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; each R₃ is independently hydrogen, alkoxy, amino, cyano, halogen, hydroxyl, or optionally substituted alkyl or aryl; each R₄ is independently hydrogen, alkoxy, amino, cyano, halogen, hydroxyl, or optionally substituted alkyl or aryl; each R₅ is independently hydrogen or optionally substituted alkyl or aryl; each R₆ is independently amino, cyano, halogen, hydrogen, OR₇, SR₇, NR₃R₉, or optionally substituted alkyl, alkyl-aryl or alkyl-heterocycle; each R₇ is independently hydrogen or optionally substituted alkyl, alkyl-aryl or alkyl-heterocycle; each R₈ is independently hydrogen or optionally substituted alkyl, alkyl-aryl or alkyl-heterocycle; each R₉ is independently hydrogen or optionally substituted alkyl, alkyl-aryl or alkyl-heterocycle; n is 1-3; and p is 1-3.

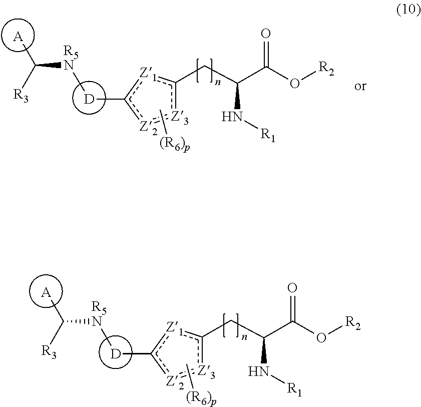

where A is optionally substituted cycloalkyl, aryl, or heterocycle; D is optionally substituted aryl or heterocycle; each of Z′₁, Z′₂, and Z′₃, is independently N, NH, S, O or CR₆; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen; R₅ is hydrogen or optionally substituted alkyl or aryl; each R₆ is independently amino, cyano, halogen, hydrogen, OR₇, SR₇, NR₈R₉, or optionally substituted alkyl, alkyl-aryl or alkyl-heterocycle; each R₇ is independently hydrogen or optionally substituted alkyl, alkyl-aryl or alkyl-heterocycle; each R₈ is independently hydrogen or optionally substituted alkyl, alkyl-aryl or alkyl-heterocycle; each R₉ is independently hydrogen or optionally substituted alkyl, alkyl-aryl or alkyl-heterocycle; and p is 1-3.

Some compounds are such that all of Z′₁, Z′₂, and Z′₃ are N or NH. In others, only two of Z′₁, Z′₂, and Z′₃ are N or NH. In others, only one of Z′₁, Z′₂, and Z′₃ is N or NH. In others, none of Z′₁, Z′₂, and Z′₃ are N or NH.

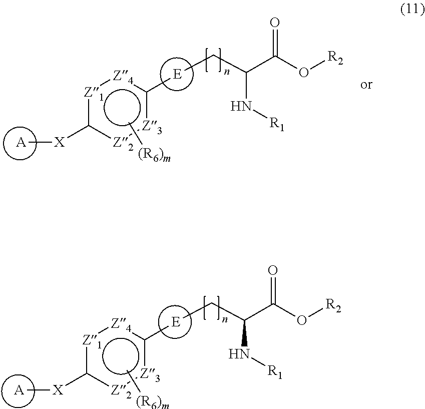

where A is optionally substituted cycloalkyl, aryl, or heterocycle; E is optionally substituted aryl or heterocycle; X is a bond, —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; each of Z″₁, Z″₂, Z″₃, and Z″₄ is independently N or CR₁₀; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; each R₃ is independently hydrogen, alkoxy, amino, cyano, halogen, hydroxyl, or optionally substituted alkyl; each R₄ is independently hydrogen, alkoxy, amino, cyano, halogen, hydroxyl, or optionally substituted alkyl or aryl; each R₅ is independently hydrogen or optionally substituted alkyl or aryl; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; each R₁₃ is independently hydrogen or optionally substituted alkyl, alkyl-aryl or alkyl-heterocycle; and n is 1-3.

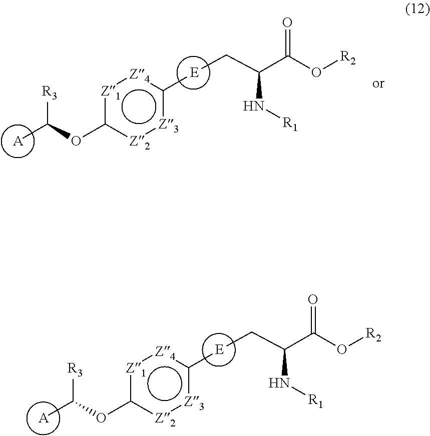

where A is optionally substituted cycloalkyl, aryl, or heterocycle; E is optionally substituted aryl or heterocycle; each of Z″₁, Z″₂, Z″₃, and Z″₄ is independently N or CR₁₀; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is trifluoromethyl; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; and each R₁₃ is independently hydrogen or optionally substituted alkyl, alkyl-aryl or alkyl-heterocycle.

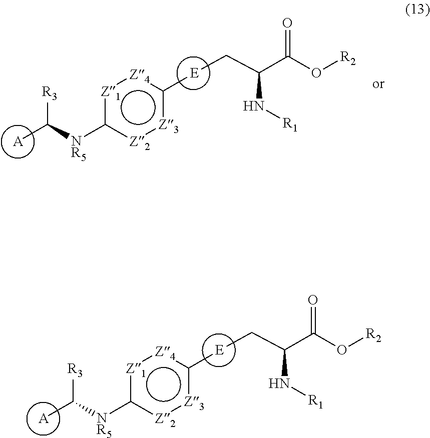

where A is optionally substituted cycloalkyl, aryl, or heterocycle; E is optionally substituted aryl or heterocycle; each of Z″₁, Z″₂, Z″₃, and Z″₄ is independently N or CR₁₀; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen; R₅ is hydrogen or optionally substituted alkyl or aryl; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; and each R₁₃ is independently hydrogen or optionally substituted alkyl, alkyl-aryl or alkyl-heterocycle.

Some compounds are such that all of Z″₁, Z″₂, Z″₃, and Z″₄ are N. In others, only three of Z″₁, Z″₂, Z″₃, and Z″₄ are N. In others, only two of Z″₁, Z″₂, Z″₃, and Z″₄ are N. In others, only one of Z″₁, Z″₂, Z″₃, and Z″₄ is N. In others, none of Z″₁, Z″₂, Z″₃, and Z″₄ are N.

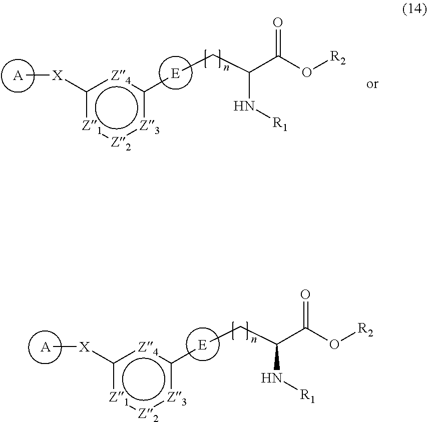

where A is optionally substituted cycloalkyl, aryl, or heterocycle; E is optionally substituted aryl or heterocycle; X is a bond, —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; each of Z″₁, Z″₂, Z″₃, and Z″₄ is independently N or CR₁₀; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; each R₃ is independently hydrogen, alkoxy, amino, cyano, halogen, hydroxyl, or optionally substituted alkyl; each R₄ is independently hydrogen, alkoxy, amino, cyano, halogen, hydroxyl, or optionally substituted alkyl or aryl; each R₅ is independently hydrogen or optionally substituted alkyl or aryl; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; each R₁₃ is independently hydrogen or optionally substituted alkyl, alkyl-aryl or alkyl-heterocycle; and n is 1-3.

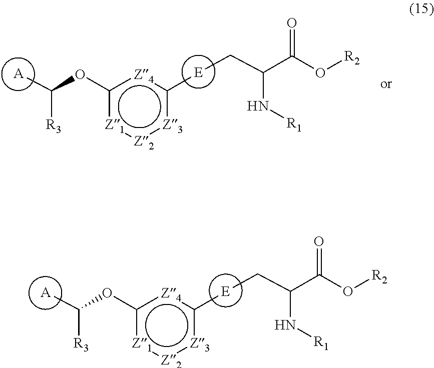

where A is optionally substituted cycloalkyl, aryl, or heterocycle; E is optionally substituted aryl or heterocycle; each of Z″₁, Z″₂, Z″₃, and Z″₄ is independently N or CR₁₀; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is trifluoromethyl; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; and each R₁₃ is independently hydrogen or optionally substituted alkyl, alkyl-aryl or alkyl-heterocycle.

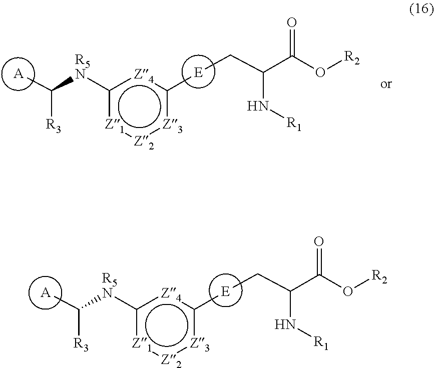

where A is optionally substituted cycloalkyl, aryl, or heterocycle; E is optionally substituted aryl or heterocycle; each of Z″₁, Z″₂, Z″₃, and Z″₄ is independently N or CR₁₀; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen; R₅ is hydrogen or optionally substituted alkyl or aryl; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; and each R₁₃ is independently hydrogen or optionally substituted alkyl, alkyl-aryl or alkyl-heterocycle.

Some compounds are such that all of Z″₁, Z″₂, Z″₃, and Z″₄ are N. In others, only three of Z″₁, Z″₂, Z″₃, and Z″ are N. In others, only two of Z″₁, Z″₂, Z″₃, and Z″₄ are N. In others, only one of Z″₁, Z″₂, Z″₃, and Z″₄ is N. In others, none of Z″₁, Z″₂, Z″₃, and Z″₄ are N.

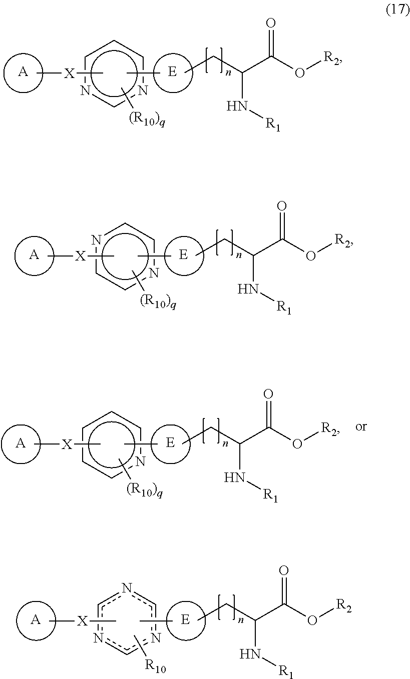

where A is optionally substituted cycloalkyl, aryl, or heterocycle; E is optionally substituted aryl or heterocycle; X is a bond, —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; each R₃ is independently hydrogen, alkoxy, amino, cyano, halogen, hydroxyl, or optionally substituted alkyl; each R₄ is independently hydrogen, alkoxy, amino, cyano, halogen, hydroxyl, or optionally substituted alkyl or aryl; each R₅ is independently hydrogen or optionally substituted alkyl or aryl; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; each R₁₃ is independently hydrogen or optionally substituted alkyl, alkyl-aryl or alkyl-heterocycle; n is 1-3; q is 0-2; and r is 0-2.

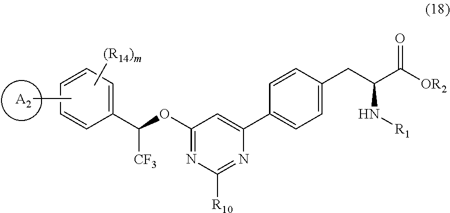

where A₂ is optionally substituted cycloalkyl, aryl, or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₁₀ is amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; R₁₂ is hydrogen or optionally substituted alkyl, alkyl-aryl or alkyl-heterocycle; R₁₃ is hydrogen or optionally substituted alkyl, alkyl-aryl or alkyl-heterocycle; each R₁₄ is independently amino, halogen, hydrogen, C(O)R_{A}, OR_{A}, NR_{B}R_{C}, S(O₂)R_{A}, or optionally substituted alkyl, alkyl-aryl or alkyl-heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; and m is 1-4.

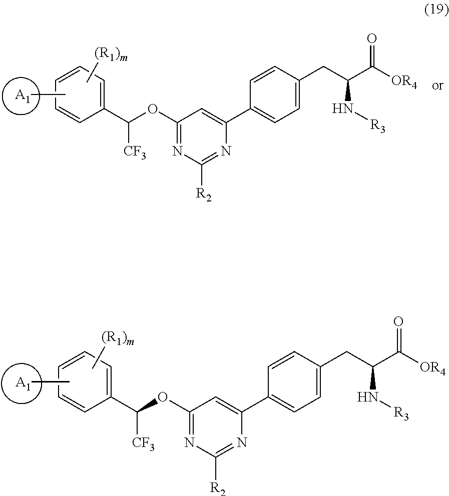

where A₁ is optionally substituted heterocycle; each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is independently halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen, C(O)R_{A}, C(O)OR_{A}, or optionally substituted alkyl, alkyl-aryl, alkyl-heterocycle, aryl, or heterocycle; R₄ is hydrogen or optionally substituted alkyl, alkyl-aryl, alkyl-heterocycle, aryl, or heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; and m is 1-4.

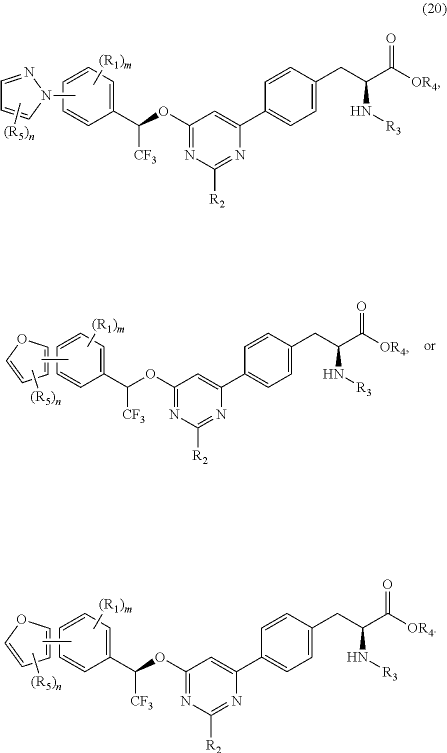

where each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is independently halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen, C(O)R_{A}, C(O)OR_{A}, or optionally substituted alkyl, alkyl-aryl, alkyl-heterocycle, aryl, or heterocycle; R₄ is hydrogen or optionally substituted alkyl, alkyl-aryl, alkyl-heterocycle, aryl, or heterocycle; each R₅ is independently halogen, hydrogen, C(O)R_{A}, OR_{A}, NR_{B}R_{C}, S(O₂)R_{A}, or optionally substituted alkyl, alkyl-aryl or alkyl-heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; m is 1-4; and n is 1-3.

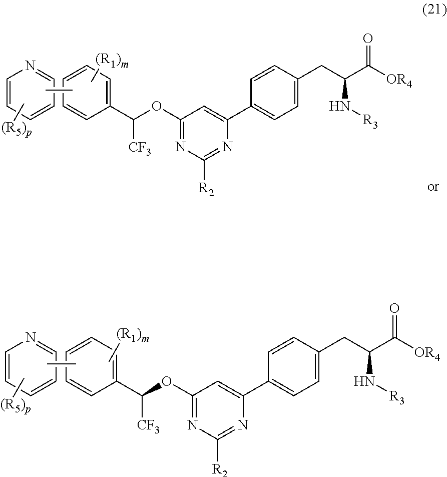

where each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is independently halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen, C(O)R_{A}, C(O)OR_{A}, or optionally substituted alkyl, alkyl-aryl, alkyl-heterocycle, aryl, or heterocycle; R₄ is hydrogen or optionally substituted alkyl, alkyl-aryl, alkyl-heterocycle, aryl, or heterocycle; each R₅ is independently halogen, hydrogen, C(O)R_{A}, OR_{A}, NR_{B}R_{C}, S(O₂)R_{A}, or optionally substituted alkyl, alkyl-aryl or alkyl-heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; m is 1-4; and p is 1-3.

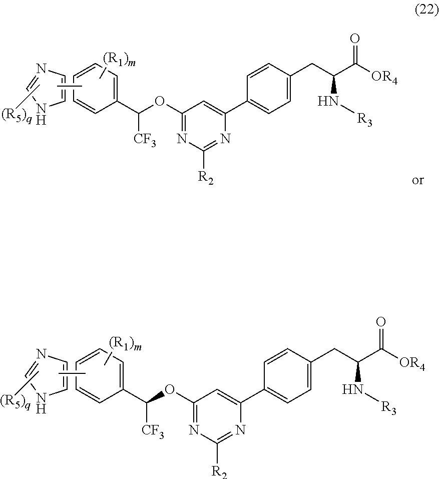

where each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is independently halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen, C(O)R_{A}, C(O)OR_{A}, or optionally substituted alkyl, alkyl-aryl, alkyl-heterocycle, aryl, or heterocycle; R₄ is hydrogen or optionally substituted alkyl, alkyl-aryl, alkyl-heterocycle, aryl, or heterocycle; each R₅ is independently halogen, hydrogen, C(O)R_{A}, OR_{A}, NR_{B}R_{C}, S(O₂)R_{A}, or optionally substituted alkyl, alkyl-aryl or alkyl-heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; m is 1-4; and q is 1-2.

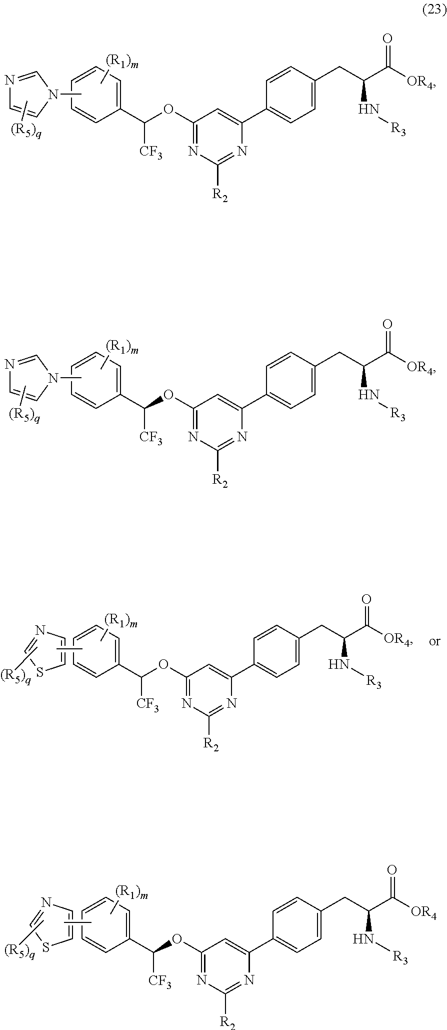

where each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is independently halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen, C(O)R_{A}, C(O)OR_{A}, or optionally substituted alkyl, alkyl-aryl, alkyl-heterocycle, aryl, or heterocycle; R₄ is hydrogen or optionally substituted alkyl, alkyl-aryl, alkyl-heterocycle, aryl, or heterocycle; each R₅ is independently halogen, hydrogen, C(O)R_{A}, OR_{A}, NR_{B}R_{C}, S(O₂)R_{A}, or optionally substituted alkyl, alkyl-aryl or alkyl-heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; m is 1-4; n is 1-3; and q is 1-2.

In particular compounds above, A₁ is aromatic. In others, A₁ is not aromatic. In some, A₁ is optionally substituted with one or more of halogen or lower alkyl.

In some, R₁ is hydrogen or halogen.

In some, m is 1.

In some, R₂ is hydrogen or amino.

In some, R₃ is hydrogen or lower alkyl. In others, R₃ is C(O)OR_{A} and R_{A} is alkyl.

In some, R₄ is hydrogen or lower alkyl.

In some, R₅ is hydrogen or lower alkyl (e.g., methyl).

In some, n is 1.

In some, p is 1.

In some, q is 1.

(24)

[symbol]

### (S)-2-Amino-3-(4-{2-amino-6-[2,2,2-trifluoro-1-(4-pyridin-4-yl-phenyl)-ethoxy]-pyrimidin-4-yl}-phenyl)-propionic acid

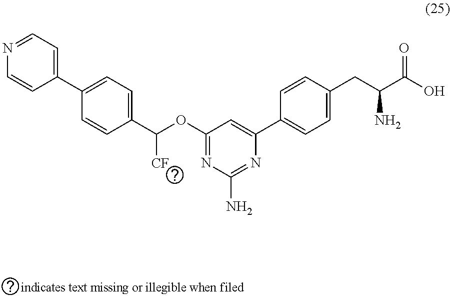

### (S)-2-Amino-3-(4-{6-[2,2,2-trifluoro-1-(2-pyridin-4-yl-phenyl)-ethoxy]-pyrimidin-4-yl}-phenyl)-propionic acid

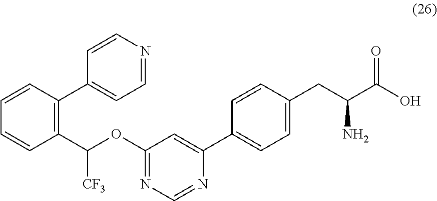 [SMILES: N[C@@H](Cc1ccc(-c2cc(OC(c3ccccc3-c3ccncc3)C(F)(F)F)ncn2)cc1)C(=O)O]

### (S)-2-Amino-3-(4-{2-amino-6-[2,2,2-trifluoro-1-(2-(4-methylthiophen-3-yl)-phenyl]-ethoxy}-pyrimidin-4-yl)-phenyl]-propionic acid

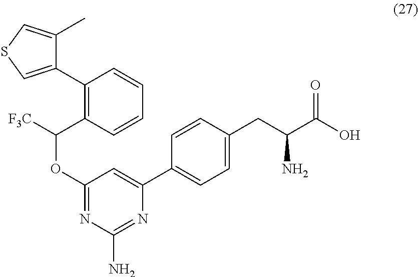 [SMILES: Cc1cscc1-c1ccccc1C(Oc1cc(-c2ccc(C[C@H](N)C(=O)O)cc2)nc(N)n1)C(F)(F)F]

### (S)-2-Amino-3-(4-{2-amino-6-[2,2,2-trifluoro-1-(2-(5-methyl-thiophen-3-yl-)-phenyl]-ethoxy}-pyrimidin-4-yl)-phenyl]-propionic acid

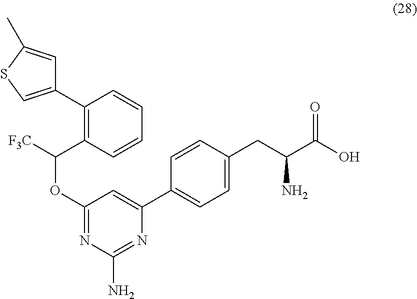 [SMILES: Cc1cc(-c2ccccc2C(Oc2cc(-c3ccc(C[C@H](N)C(=O)O)cc3)nc(N)n2)C(F)(F)F)cs1]

### (S)-2-Amino-3-(4-{2-amino-6-[2,2,2-trifluoro-1-(4-furan-3-yl-phenyl)-ethoxy]-pyrimidin-4-yl}-phenyl)-propionic acid

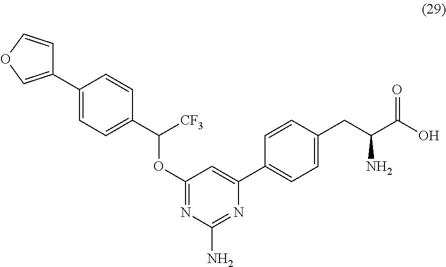 [SMILES: Nc1nc(OC(c2ccc(-c3ccoc3)cc2)C(F)(F)F)cc(-c2ccc(C[C@H](N)C(=O)O)cc2)n1]

### (S)-2-Amino-3-[4-{2-amino-6-{1-[2-(5-dimethylaminomethyl-furan-2-yl)-phenyl]-2,2,2-trifluoro-ethoxy}-pyrimidin-4-yl)-phenyl]-propionic acid

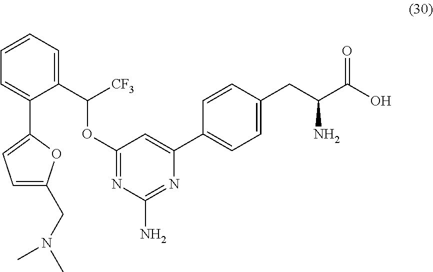 [SMILES: CN(C)Cc1ccc(-c2ccccc2C(Oc2cc(-c3ccc(C[C@H](N)C(=O)O)cc3)nc(N)n2)C(F)(F)F)o1]

### (S)-2-Amino-3[4-(2-amino-6-{1-[2-(6-cyano-pyridin-3-yl)-phenyl]-2,2,2-tri-fluoro-ethoxy}-pyrimidin-4-yl)-phenyl]-propionic acid

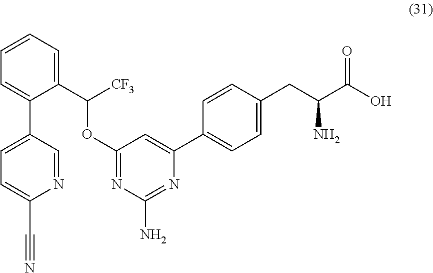 [SMILES: N#Cc1ccc(-c2ccccc2C(Oc2cc(-c3ccc(C[C@H](N)C(=O)O)cc3)nc(N)n2)C(F)(F)F)cn1]

### (S)-2-Amino-3-(4-{2-amino-6-[2,2,2-trifluoro-1-(2-imidazol-1-yl-phenyl)-ethoxy]-pyrimidin-4-yl}-phenyl)-propionic acid

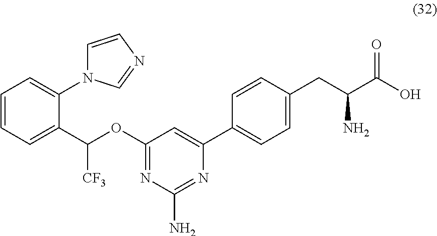 [SMILES: Nc1nc(OC(c2ccccc2-n2ccnc2)C(F)(F)F)cc(-c2ccc(C[C@H](N)C(=O)O)cc2)n1]

### (S)-2-Amino-3-(4-{6-[2,2,2-trifluoro-1-(2-pyrazol-1-yl-phenyl)-ethoxy]-pyrimidin-4-yl}-phenyl)-propionic acid

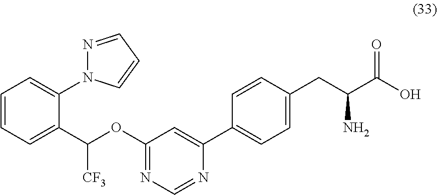 [SMILES: N[C@@H](Cc1ccc(-c2cc(OC(c3ccccc3-n3cccn3)C(F)(F)F)ncn2)cc1)C(=O)O]

### (S)-2-amino-3-[4-(2-amino-6-{2,2,2-trifluoro-1-[2-(3-trifluoromethyl-pyrazol-1-yl)-phenyl]-ethoxy}-pyrimidin-4-yl)-phenyl]-propionic acid

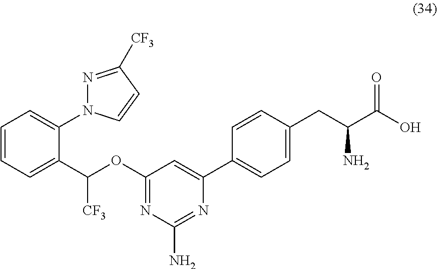 [SMILES: Nc1nc(OC(c2ccccc2-n2ccc(C(F)(F)F)n2)C(F)(F)F)cc(-c2ccc(C[C@H](N)C(=O)O)cc2)n1]

### (S)-2-Amino-3-[4-(2-amino-6-{1-[2-(3,5-dimethyl-pyrazol-1-yl)-phenyl]-2,2,2-trifluoro-ethoxy}-pyrimidin-4-yl)-phenyl]-propionic acid

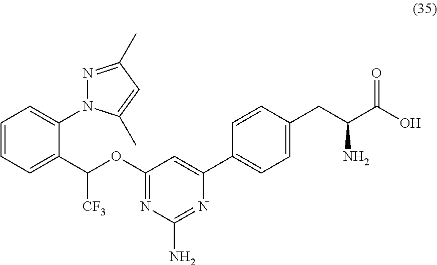 [SMILES: Cc1cc(C)n(-c2ccccc2C(Oc2cc(-c3ccc(C[C@H](N)C(=O)O)cc3)nc(N)n2)C(F)(F)F)n1]

### (S)-2-Amino-3-[4-(2-amino-6-{2,2,2-trifluoro-1-[2-(3-phenyl-pyrazol-1-yl)-phenyl]-ethoxy}-pyrimidin-4-yl)-phenyl]-propionic acid

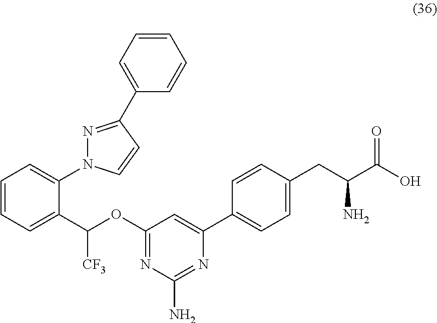 [SMILES: Nc1nc(OC(c2ccccc2-n2ccc(-c3ccccc3)n2)C(F)(F)F)cc(-c2ccc(C[C@H](N)C(=O)O)cc2)n1]

### (S)-2-Amino-3-[4-(2-amino-6-{2,2,2-trifluoro-1-[5-methoxy-2-(4-methyl-pyrazol-1-yl)-phenyl]-ethoxy}-pyrimidin-4-yl)-phenyl]-propionic acid

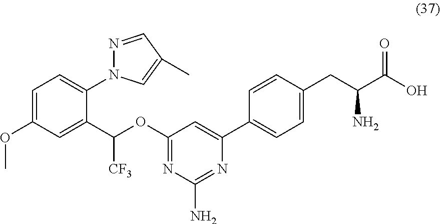 [SMILES: COc1ccc(-n2cc(C)cn2)c(C(Oc2cc(-c3ccc(C[C@H](N)C(=O)O)cc3)nc(N)n2)C(F)(F)F)c1]

### (S)-2-amino-3-[4-(2-amino-6-{(R)-2,2,2-trifluoro-1-[2-(3-methyl-pyrazol-1-yl)-phenyl]-ethoxy}-pyrimidin-4-yl)-phenyl]-propionic acid

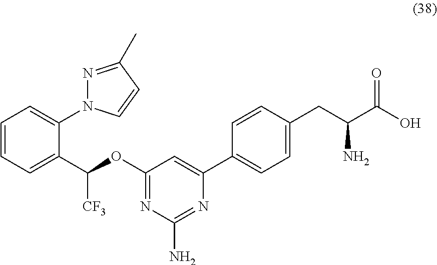 [SMILES: Cc1ccn(-c2ccccc2[C@@H](Oc2cc(-c3ccc(C[C@H](N)C(=O)O)cc3)nc(N)n2)C(F)(F)F)n1]

### (S)-2-amino-3-[4-(2-amino-6-{1-[4-chloro-2-(3-methyl-pyrazol-1-yl)-phenyl-]-2,2,2-trifluoro-ethoxy}-pyrimidin-4-yl)-phenyl]-propionic acid

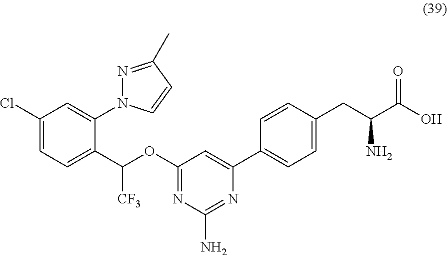 [SMILES: Cc1ccn(-c2cc(Cl)ccc2C(Oc2cc(-c3ccc(C[C@H](N)C(=O)O)cc3)nc(N)n2)C(F)(F)F)n1]

### (S)-2-Amino-3-[4-(2-amino-6-{R-1-[4-chloro-2-(3-methyl-pyrazol-1-yl)-phenyl]-2,2,2-trifluoro-ethoxy}-pyrimidin-4-yl)-phenyl]-propionic acid ethyl ester

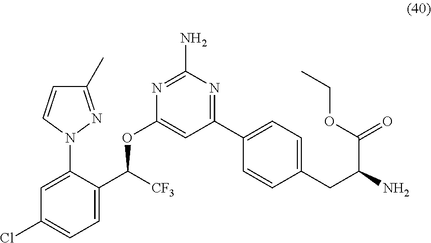 [SMILES: CCOC(=O)[C@@H](N)Cc1ccc(-c2cc(O[C@H](c3ccc(Cl)cc3-n3ccc(C)n3)C(F)(F)F)nc(N)n2)cc1]

### (S)-2-amino-3-(4-(2-amino-6-((R)-1-(4-chloro-2-(3-methyl-1H-pyrazol-1-yl)-phenyl)-2,2,2-trifluoroethoxy)pyrimidin-4-yl)phenyl)propanoic acid

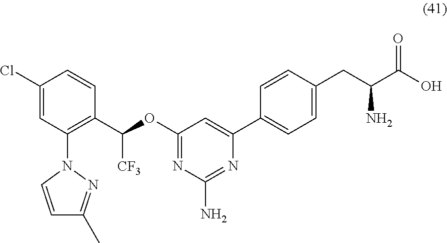 [SMILES: Cc1ccn(-c2cc(Cl)ccc2[C@@H](Oc2cc(-c3ccc(C[C@H](N)C(=O)O)cc3)nc(N)n2)C(F)(F)F)n1]

### (S)-2-Amino-3-(4-{2-amino-6-[2,2,2-trifluoro-1-(2-thiazol-2-yl-phenyl)-ethoxy]-pyrimidin-4-yl}-phenyl)-propionic acid

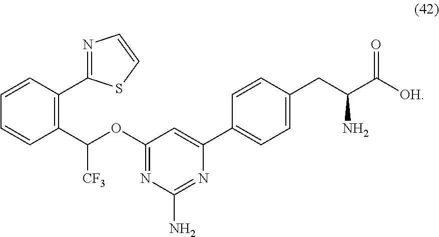 [SMILES: Nc1nc(OC(c2ccccc2-c2nccs2)C(F)(F)F)cc(-c2ccc(C[C@H](N)C(=O)O)cc2)n1]

### (S)-2-Amino-3-[4-(2-amino-6-{2,2,2-trifluoro-1-[2-(pyridin-3-yloxy)-phenyl-1}-ethoxy]-pyrimidin-4-yl)-phenyl]-propionic acid

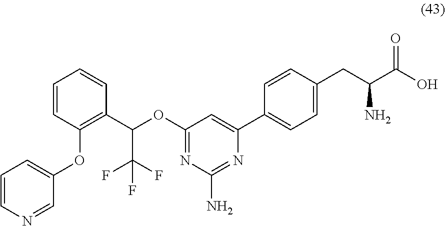 [SMILES: Nc1nc(OC(c2ccccc2Oc2cccnc2)C(F)(F)F)cc(-c2ccc(C[C@H](N)C(=O)O)cc2)n1]

### (S)-2-Amino-3-[4-(2-amino-6-{2,2,2-trifluoro-1-[4-(pyridin-3-yloxy)-phenyl]-ethoxy}-pyrimidin-4-yl)-phenyl]-propionic acid

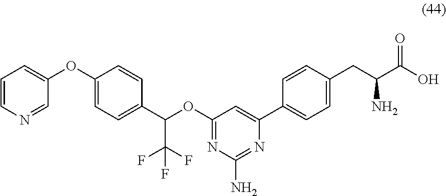 [SMILES: Nc1nc(OC(c2ccc(Oc3cccnc3)cc2)C(F)(F)F)cc(-c2ccc(C[C@H](N)C(=O)O)cc2)n1]

### (S)-2-Amino-3-[4-(6-{2,2,2-trifluoro-1-[4-(pyridin-3-yloxy)-phenyl]-ethoxy}-pyrimidin-4-yl)-phenyl]-propionic acid

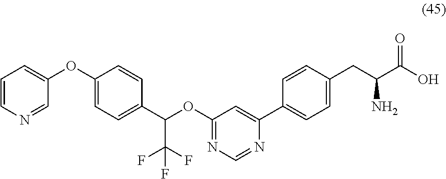 [SMILES: N[C@@H](Cc1ccc(-c2cc(OC(c3ccc(Oc4cccnc4)cc3)C(F)(F)F)ncn2)cc1)C(=O)O]

i

### (S)-2-Amino-3-(4-{2-amino-6-[2,2,2-trifluoro-1-(4-thiophen-2-yl-phenyl)-ethoxy]-pyrimidin-4-yl}-phenyl)-propionic acid

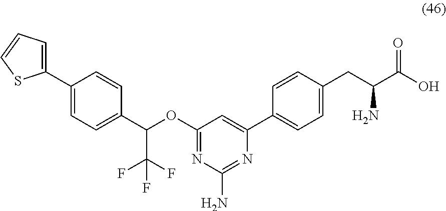 [SMILES: Nc1nc(OC(c2ccc(-c3cccs3)cc2)C(F)(F)F)cc(-c2ccc(C[C@H](N)C(=O)O)cc2)n1]

### (S)-2-Amino-3-(4-{6-[2,2,2-trifluoro-1-(4-imidazol-1-yl-phenyl)-ethoxy]-pyrimidin-4-yl}-phenyl)-propionic acid

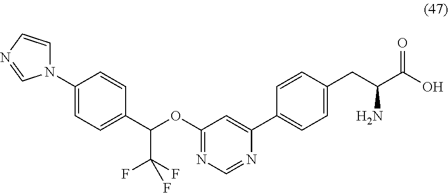 [SMILES: N[C@@H](Cc1ccc(-c2cc(OC(c3ccc(-n4ccnc4)cc3)C(F)(F)F)ncn2)cc1)C(=O)O]

### (S)-2-Amino-3-(4-{2-amino-6-[2,2,2-trifluoro-1-(4-[1,2,4]triazol-1-yl-phenyl)-ethoxy]-pyrimidin-4-yl}-phenyl)-propionic acid

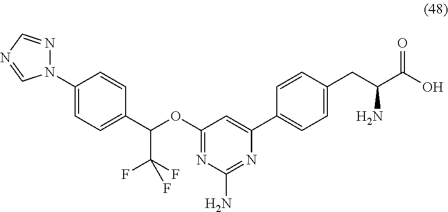 [SMILES: Nc1nc(OC(c2ccc(-n3cncn3)cc2)C(F)(F)F)cc(-c2ccc(C[C@H](N)C(=O)O)cc2)n1]

### (S)-2-Amino-3-(4-{2-amino-6-[2,2,2-trifluoro-1-(4-fluoro-2-thiophen-3-yl-phenyl)ethoxy]-pyrimidin-4-yl}-phenyl)-propionic acid

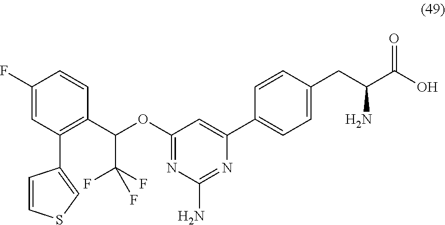 [SMILES: Nc1nc(OC(c2ccc(F)cc2-c2ccsc2)C(F)(F)F)cc(-c2ccc(C[C@H](N)C(=O)O)cc2)n1]

### (S)-2-Amino-3-[4-(2-amino-6-{2,2,2-trifluoro-1-[4-fluoro-2-(4-methyl-thiophen-2-yl)-phenyl]-ethoxy}-pyrimidin-4-yl)-phenyl]-propionic acid

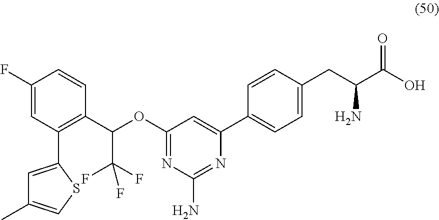 [SMILES: Cc1csc(-c2cc(F)ccc2C(Oc2cc(-c3ccc(C[C@H](N)C(=O)O)cc3)nc(N)n2)C(F)(F)F)c1]

### (S)-2-Amino-3-[4-(2-amino-6-{1-[2-(3,5-dimethyl-isoxazol-4-yl)-4-fluoro-phenyl]-2,2,2-trifluoro-ethoxy}-pyrimidin-4-yl)-phenyl]-propionic acid

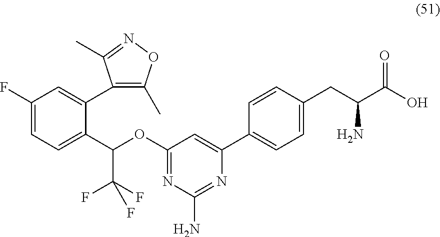 [SMILES: Cc1noc(C)c1-c1cc(F)ccc1C(Oc1cc(-c2ccc(C[C@H](N)C(=O)O)cc2)nc(N)n1)C(F)(F)F]

### (S)-2-amino-3-[4-(2-amino-6-{2,2,2-trifluoro-1-[5-fluoro-2-(3-methyl-pyrazol-1-yl)-phenyl]-ethoxy}-pyrimidin-4-yl)-phenyl]-propionic acid

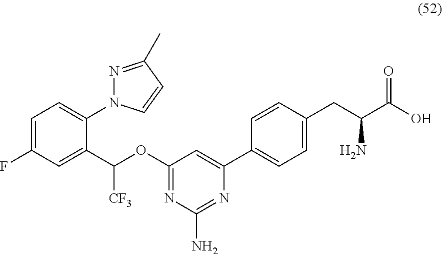 [SMILES: Cc1ccn(-c2ccc(F)cc2C(Oc2cc(-c3ccc(C[C@H](N)C(=O)O)cc3)nc(N)n2)C(F)(F)F)n1]

### (S)-2-amino-3-[4-(2-amino-6{2,2,2-trifluoro-1-[5-chloro-2-(3-methyl-pyrazol-1-yl)-phenyl]-ethoxy}-pyrimidin-4-yl)-phenyl]-propionic acid

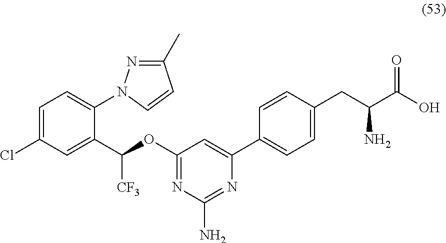 [SMILES: Cc1ccn(-c2ccc(Cl)cc2[C@@H](Oc2cc(-c3ccc(C[C@H](N)C(=O)O)cc3)nc(N)n2)C(F)(F)F)n1]

### (S)-2-amino-3-[4-(2-amino-6-{2,2,2-trifluoro-1-[4-(2-oxo-pyrrolidin-1-yl)-phenyl]-ethoxy}-pyrimidin-4-yl)-phenyl]-propionic acid

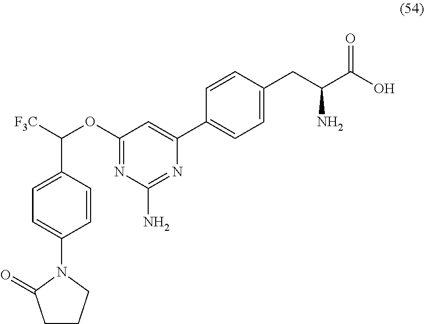 [SMILES: Nc1nc(OC(c2ccc(N3CCCC3=O)cc2)C(F)(F)F)cc(-c2ccc(C[C@H](N)C(=O)O)cc2)n1]

### (S)-2-Amino-3-[4-(2-amino-6-{(R)-2,2,2-trifluoro-1-[5-fluoro-2-(3-methyl-pyrazol-1-yl)-phenyl]ethoxy}-pyrimidin-4-yl)-phenyl]-propionic acid

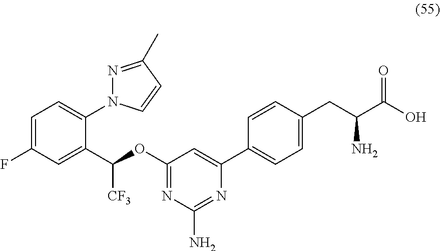 [SMILES: Cc1ccn(-c2ccc(F)cc2[C@@H](Oc2cc(-c3ccc(C[C@H](N)C(=O)O)cc3)nc(N)n2)C(F)(F)F)n1]

### (S)-2-Amino-3-[4-(2-amino-6-{2,2,2-trifluoro-1-[4-(6-methoxy-pyridin-2-yl)-phenyl]-ethoxy}-pyrimidin-4-yl)-phenyl]-propionic acid

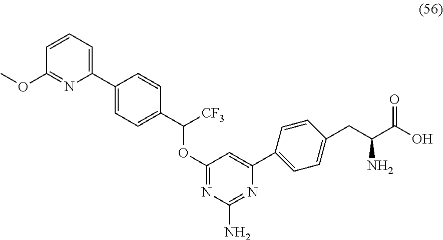 [SMILES: COc1cccc(-c2ccc(C(Oc3cc(-c4ccc(C[C@H](N)C(=O)O)cc4)nc(N)n3)C(F)(F)F)cc2)n1]

### (S)-2-Amino-3-[4-(2-amino-6-{2,2,2-trifluoro-1-[2-fluoro-4-(5-methoxy-pyridin-3-yl)-phenyl]-ethoxy}-pyrimidin-4-yl)-phenyl]-propionic acid

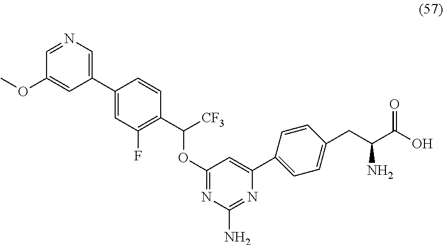 [SMILES: COc1cncc(-c2ccc(C(Oc3cc(-c4ccc(C[C@H](N)C(=O)O)cc4)nc(N)n3)C(F)(F)F)c(F)c2)c1]

### (S)-2-Amino-3-[4-(2-amino-6-{(S)-2,2,2-trifluoro-1-[4-(2-fluoro-pyridin-4-yl)-phenyl]-ethoxy}-pyrimidin-4-yl)-phenyl]-propionic acid

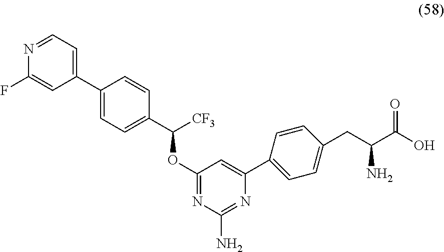 [SMILES: Nc1nc(O[C@@H](c2ccc(-c3ccnc(F)c3)cc2)C(F)(F)F)cc(-c2ccc(C[C@H](N)C(=O)O)cc2)n1]

### (S)-2-Amino-3-[4-(2-amino-6-{(S)-2,2,2-trifluoro-1-[4-(5-methoxy-pyridin-3-yl)-phenyl]-ethoxy}-pyrimidin-4-yl)-phenyl]-propionic acid

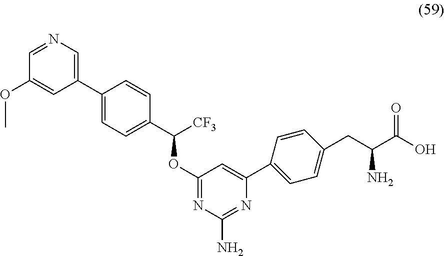 [SMILES: COc1cncc(-c2ccc([C@H](Oc3cc(-c4ccc(C[C@H](N)C(=O)O)cc4)nc(N)n3)C(F)(F)F)cc2)c1]

### (S)-2-Amino-3-[4-(2-amino-6-{(S)-2,2,2-trifluoro-1-[4-(4-trifluoromethyl-pyridin-3-yl)-phenyl]-ethoxy}-pyrimidin-4-yl)-phenyl]-propionic acid

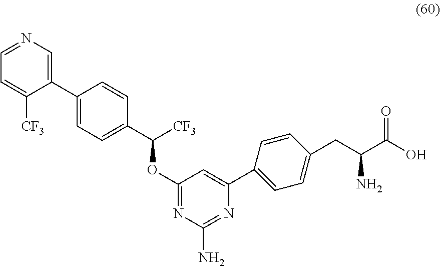 [SMILES: Nc1nc(O[C@@H](c2ccc(-c3cnccc3C(F)(F)F)cc2)C(F)(F)F)cc(-c2ccc(C[C@H](N)C(=O)O)cc2)n1]

### (S)-2-Amino-3-(4-{2-amino-6-[(S)-2,2,2-trifluoro-1-(4-isoxazol-4-yl-pheny-1)-ethoxy]-pyrimidin-4-yl}-phenyl)-propionic acid

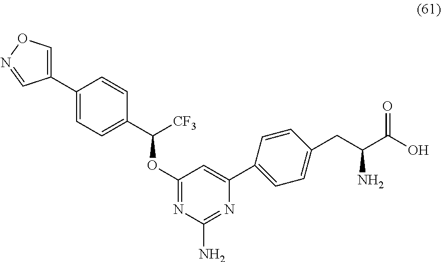 [SMILES: Nc1nc(O[C@@H](c2ccc(-c3cnoc3)cc2)C(F)(F)F)cc(-c2ccc(C[C@H](N)C(=O)O)cc2)n1]

### (S)-2-Amino-3-(4-{2-amino-6-[2,2,2-trifluoro-1-(2-pyrimidin-5-yl-phenyl)-ethoxy]-pyrimidin-4-yl}-phenyl)-propionic acid

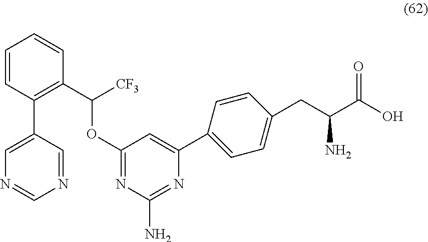 [SMILES: Nc1nc(OC(c2ccccc2-c2cncnc2)C(F)(F)F)cc(-c2ccc(C[C@H](N)C(=O)O)cc2)n1]

### (S)-2-amino-3-(4-{2-amino-6-[2,2,2-trifluoro-1-(2-thiophen-3-yl-phenyl)-ethoxy]-pyrimidin-4-yl}-phenyl)-propionic acid

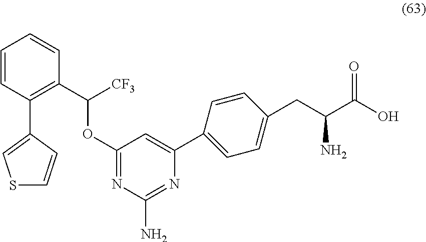 [SMILES: Nc1nc(OC(c2ccccc2-c2ccsc2)C(F)(F)F)cc(-c2ccc(C[C@H](N)C(=O)O)cc2)n1]

### (S)-2-Amino-3-[4-(2-amino-6-{2,2,2-trifluoro-1-[2-(1-methyl-1H-pyrazol-4-yl)-phenyl]-ethoxy}-pyrimidin-4-yl)-phenyl]-propionic acid

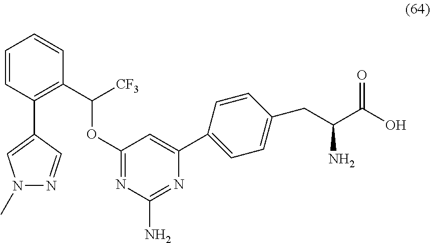 [SMILES: Cn1cc(-c2ccccc2C(Oc2cc(-c3ccc(C[C@H](N)C(=O)O)cc3)nc(N)n2)C(F)(F)F)cn1]

### (S)-2-amino-3-(4-{6-[2,2,2-trifluoro-1-(2-furan-3-yl-phenyl)-ethoxy]-pyrimidin-4-yl}-phenyl)-propionic acid

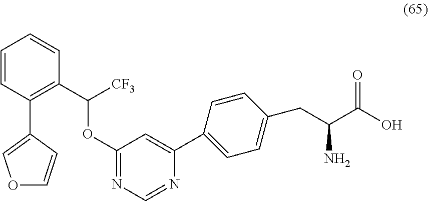 [SMILES: N[C@@H](Cc1ccc(-c2cc(OC(c3ccccc3-c3ccoc3)C(F)(F)F)ncn2)cc1)C(=O)O]

### (S)-2-amino-3-(4-{6-[2,2,2-trifluoro-1-(2-furan-2-yl-phenyl)-ethoxy]-pyrimidin-4-yl}-phenyl)-propionic acid

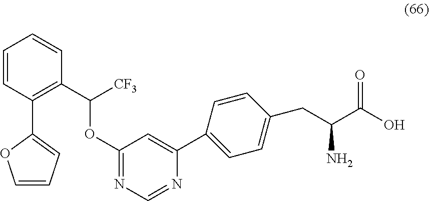 [SMILES: N[C@@H](Cc1ccc(-c2cc(OC(c3ccccc3-c3ccco3)C(F)(F)F)ncn2)cc1)C(=O)O]

### (2S)-2-amino-3-(4-(2-amino-6-(2,2,2-trifluoro-1-(4-(pyridin-3-yl)phenyl)ethoxy)pyrimidin-4-yl)phenyl)propanoic acid

### (67) (2S)-2-amino-3-(4-(2-amino-6-(2,2,2-trifluoro-1-(2-(2-methylpyridin-4-yl)phenyl)ethoxy)pyrimidin-4-yl)phenyl)propanoic acid

### (68) (2S)-2-amino-3-(4-(2-amino-6-(2,2,2-trifluoro-1-(2-(4-methylthiophen-3-yl)phenyl)ethoxy)pyrimidin-4-yl)phenyl)propanoic acid

### (69) (2S)-3-(4-(6-(1-(2-(1H-pyrazol-1-yl)phenyl)-2,2,2-trifluoroethoxy)-2-aminopyrimidin-4-yl)phenyl)-2-aminopropanoic acid

### (70) (2S)-2-amino-3-(4-(2-amino-6-(2,2,2-trifluoro-1-(4-(furan-2-yl)phenyl)ethoxy)pyrimidin-4-yl)phenyl)propanoic acid

### (71) (2S)-2-amino-3-(4-(6-(2,2,2-trifluoro-1-(2-(pyridin-3-yloxy)phenyl)ethoxy)pyrimidin-4-yl)phenyl)propanoic acid

### (72) (2S)-3-(4-(6-(1-(2-(1H-1,2,4-triazol-1-yl)phenyl)-2,2,2-trifluoroethoxy)-2-aminopyrimidin-4-yl)phenyl)-2-aminopropanoic acid

### (73) (2S)-2-amino-3-(4-(2-amino-6-(2,2,2-trifluoro-1-(2-(furan-3-yl)phenyl)ethoxy)pyrimidin-4-yl)phenyl)propanoic acid

### (74) (2S)-2-amino-3-(4-(2-amino-6-(2,2,2-trifluoro-1-(4-(furan-2-yl)-3-methoxyphenyl)ethoxy)pyrimidin-4-yl)phenyl)propanoic acid

### (75) (2S)-2-amino-3-(4-(5-(2,2,2-trifluoro-1-(2-(furan-2-yl)phenyl)ethoxy)pyrazin-2-yl)phenyl)propanoic acid

### (76) (2S)-3-(4-(5-(1-(2-(1H-pyrazol-1-yl)phenyl)-2,2,2-trifluoroethoxy)pyrazin-2-yl)phenyl)-2-aminopropanoic acid

### (77) (2S)-2-amino-3-(4-(2-amino-6-(1-(4,5-dimethoxy-2-(1H-pyrazol-1-yl)phenyl)-2,2,2-trifluoroethoxy)pyrimidin-4-yl)phenyl)propanoic acid

### (78) (2S)-2-amino-3-(4-(2-amino-6-(2,2,2-trifluoro-1-(2-(2-methyl-1H-imidazol-1-yl)phenyl)ethoxy)pyrimidin-4-yl)phenyl)propanoic acid

### (79) (2S)-2-amino-3-(4-(2-amino-6-(2,2,2-trifluoro-1-(2-(5-methylthiophen-2-yl)phenyl)ethoxy)pyrimidin-4-yl)phenyl)propanoic acid

### (80) (2S)-2-amino-3-(4-(2-amino-6-(1-(2-(5-(dimethylcarbamoyl)furan-2-yl)phenyl)-2,2,2-trifluoroethoxy)pyrimidin-4-yl)phenyl)propanoic acid

### (81) (2S)-2-amino-3-(4-(2-amino-6-(2,2,2-trifluoro-1-(4-fluoro-2-(thiophen-2-yl)phenyl)ethoxy)pyrimidin-4-yl)phenyl)propanoic acid

### (82) (2S)-2-amino-3-(4-(6-(2,2,2-trifluoro-1-(4-fluoro-2-(thiophen-2-yl)phenyl)ethoxy)pyrimidin-4-yl)phenyl)propanoic acid

### (83) (2S)-2-amino-3-(4-(6-(2,2,2-trifluoro-1-(4-fluoro-2-(thiophen-3-yl)phenyl)ethoxy)pyrimidin-4-yl)phenyl)propanoic acid

### (84) (2S)-2-amino-3-(4-(6-(2,2,2-trifluoro-1-(4-fluoro-2-(4-methylthiophen-2-yl)phenyl)ethoxy)pyrimidin-4-yl)phenyl)propanoic acid

### (85) (S)-2-amino-3-(4-(2-amino-6-((R)-2,2,2-trifluoro-1-(4-(6-fluoropyridin-3-yl)phenyl)ethoxy)pyrimidin-4-yl)phenyl)propanoic acid

### (86) (2S)-3-(4-(6-(1-(4-(1H-imidazol-1-yl)phenyl)-2,2,2-trifluoroethoxy)-2-aminopyrimidin-4-yl)phenyl)-2-aminopropanoic acid

### (87) (2S)-2-amino-3-(4-(6-(2,2,2-trifluoro-1-(4-(thiophen-2-yl)phenyl)ethoxy)pyrimidin-4-yl)phenyl)propanoic acid

### (88) (S)-2-amino-3-(4-(2-amino-6-((R)-2,2,2-trifluoro-1(4-(pyrimidin-5-yl)phenyl)ethoxy)pyrimidin-4-yl)phenyl)propanoic acid

### (89) (2S)-2-amino-3-(4-(6-(1-(2-(3,5-dimethylisoxazol-4-yl)-4-fluorophenyl)-2,2,2-trifluoroethoxy)pyrimidin-4-yl)phenyl)propanoic acid

### (90) (S)-2-amino-3-(4-(2-amino-6-((R)-2,2,2-trifluoro-1-(4-(2-methylpyridin-4-yl)phenyl)ethoxy)pyrimidin-4-yl)phenyl)propanoic acid

### (91) (2S)-3-(4-(6-(1-(4-(1H-1,2,4-triazol-1-yl)phenyl)-2,2,2-trifluoroethoxy)pyrimidin-4-yl)phenyl)-2-aminopropanoic acid

### (92) (2S)-2-amino-3-(4-(2-amino-6-(2,2,2-trifluoro-1-(4-(piperidin-1-ylmethyl)phenyl)ethoxy)pyrimidin-4-yl)phenyl)propanoic acid

### (93) (2S)-2-amino-3-(4-(2-amino-6-(2,2,2-trifluoro-1-(2-fluoro-4-(2-methylpyridin-4-yl)phenyl)ethoxy)pyrimidin-4-yl)phenyl)propanoic acid

### (94) (2S)-2-amino-3-(4-(2-amino-6-(1-(4-(6-chloropyridazin-3-yl)phenyl)-2,2,2-trifluoroethoxy)pyrimidin-4-yl)phenyl)propanoic acid

### (95) (2S)-2-amino-3-(4-(2-amino-6-(1-(4-(4-tert-butylthiazol-2-yl)phenyl)-2,2,2-trifluoroethoxy)pyrimidin-4-yl)phenyl)propanoic acid

### (96) (2S)-2-amino-3-(4-(2-amino-6-(2,2,2-trifluoro-1-(3′-methoxy-3-(3-methyl-1H-pyrazol-1-yl)biphenyl-4-yl)ethoxy)pyrimidin-4-yl)phenyl)propanoic acid

### (97) (2S)-2-amino-3-(4-(2-amino-6-(1-(5-chloro-2-(3-methyl-1H-pyrazol-1-yl)phenyl)-2,2,2-trifluoroethoxy)pyrimidin-4-yl)phenyl)propanoic acid

### (98) (S)-2-amino-3-(4-(2-amino-6-((R)-2,2,2-trifluoro-1-(3′-methoxybiphenyl-4-yl)ethoxy)pyrimidin-4-yl)phenyl)propanoate tosylate.

### (99) (S)-2-amino-3-(4-(2-amino-6-((R)-2,2,2-trifluoro-1-(3′-methoxybiphenyl-4-yl)ethoxy)pyrimidin-4-yl)phenyl)propanoate maleate

### (100) (S)-ethyl 2-amino-3-(4-(2-amino-6-((R)-1-(4-chloro-2-(3-methyl-1-H-pyrazol-1-yl)phenyl)-2,2,2-trifluoroethoxy)pyrimidin-4-yl)phenyl)propanoate hippurate

### (101) (S)-ethyl 2-amino-3-(4-(2-amino-6-((R)-1-(4-chloro-2-(3-methyl-1-H-pyrazol-1-yl)phenyl)-2,2,2-trifluoroethoxy)pyrimidin-4-yl)phenyl)propanoate succinate

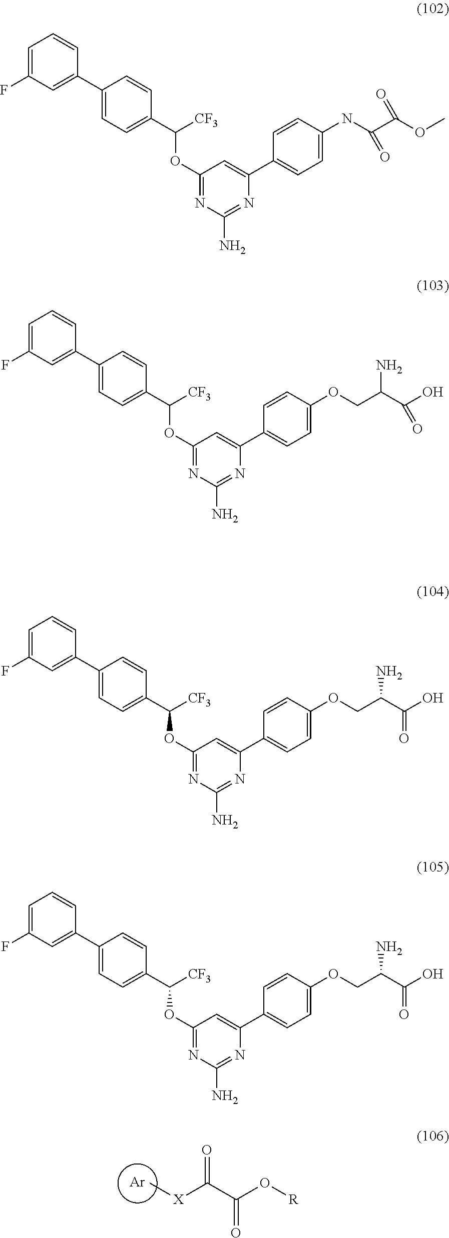

where Ar is a structure comprising multiple aryl or heterocycle rings;

### X is —CH₂— or N; and

R is hydrogen or optionally substituted alkyl, alkyl-aryl, alkyl-heterocycle, aryl, or heterocycle.

In some compounds, X is N.

In some compounds, R is methyl, ethyl, or isopropyl.

In some compounds, X is N and R is methyl.

In some compounds, Ar is a structure comprising 1-4 optionally substituted linked cycloalkyl, aryl, or heterocycle rings. In some compounds, Ar comprises 1 ring; in some compounds, Ar comprises 2 rings; in some compounds, Ar comprises 3 rings; in some compounds, Ar comprises 4 rings.

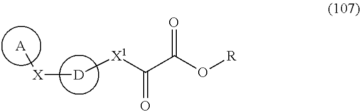

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; R is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; and X¹ is —CH₂— or N.

In some compounds, X¹ is N.

In some compounds, R is methyl, ethyl, or isopropyl.

In some compounds, X¹ is N and R is methyl.

In some compounds, X¹ is N, R is methyl, X is —C(R₃R₄)O—, R₃ is hydrogen, and R₄ is substituted alkyl.

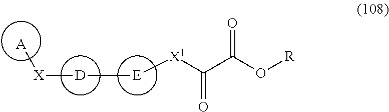

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; E is optionally substituted aryl or heterocycle; R is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; and X¹ is —CH₂— or N.

In some compounds, X¹ is N.

In some compounds, R is methyl, ethyl, or isopropyl.

In some compounds, X¹ is N and R is methyl.

In some compounds, A is fluoro-substituted biphenyl, X¹ is N, R is methyl, X is —C(R₃R₄)O—, R₃ is hydrogen, and R₄ is substituted alkyl. In some of these compounds, A is 3′-fluorobiphenyl. In some of these compounds, R₄ is halo-substituted methyl. In some of these compounds, D is substituted pyrimidinyl and E is phenyl. In some of these compounds, D is 2-substituted pyrimidinyl.

In some compounds, A is optionally substituted biphenyl, X is —C(R₃R₄)O—, R₃ is hydrogen, R₄ is optionally substituted lower alkyl, D is optionally substituted pyrimidinyl, E is phenyl, X¹ is N, and R is lower alkyl. In some of these compounds, A is 2′-, 3′-, 4′-, 5′, or 6′-halo-substituted biphenyl, X is —C(R₃R₄)O—, R₃ is hydrogen, R₄ is halo-substituted methyl or ethyl, D is 2-substituted pyrimidinyl, E is phenyl, X¹ is N, and R is methyl or ethyl. In some of these compounds, A is 2′-, 3′-, 4′-, 5′, or 6′-fluoro-substituted biphenyl, X is —C(R₃R₄)O—, R₃ is hydrogen, R₄ is fluoro-substituted methyl, D is 2-amino substituted pyrimidinyl, E is phenyl, X¹ is N, and R is methyl.

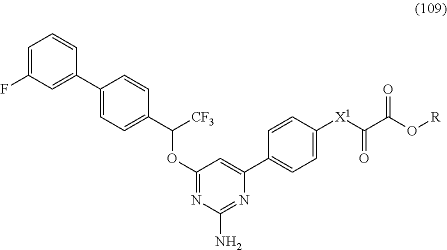

where: X¹ is —CH₂— or N; and
R is hydrogen or optionally substituted alkyl, alkyl-aryl, alkyl-heterocycle, aryl, or heterocycle.

In some compounds, X¹ is N.

In some compounds, R is methyl, ethyl, or isopropyl.

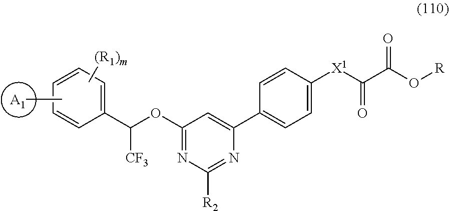

where A₁ is optionally substituted aryl or heterocycle; each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is independently halogen, hydrogen, C(O)R_{A}, OR_{A}, NR_{B}R_{C}, S(O₂)R_{A}, or optionally substituted alkyl, alkyl-aryl or alkyl-heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; X¹ is —CH₂— or N; R is hydrogen or optionally substituted alkyl, alkyl-aryl, alkyl-heterocycle, aryl, or heterocycle; and m is 1-4.

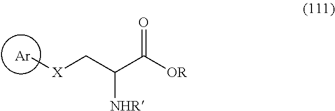

where:
Ar is a structure comprising multiple aryl or heterocycle rings;

### X is N, O, or S;

R is hydrogen or optionally substituted alkyl, alkyl-aryl, alkyl-heterocycle, aryl, or heterocycle; and
R′ is hydrogen or optionally substituted alkyl.

In some compounds, X is O.

In some compounds, X is O and R and R′ are hydrogen.

In some compounds, Ar is a structure comprising 1-4 optionally substituted linked cycloalkyl, aryl, or heterocycle rings. In some compounds, Ar comprises 1 ring; in some compounds, Ar comprises 2 rings; in some compounds, Ar comprises 3 rings; in some compounds, Ar comprises 4 rings.

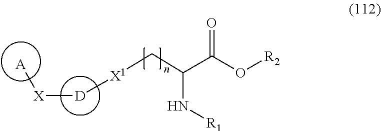

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; R₁ is hydrogen or alkyl; R₂ is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; n is 0-3; and X¹ is N, O, or S. In certain embodiments, the carbon having the HNR¹ group is in the S configuration.

In some compounds, X¹ is O and n is 1.

In some compounds, X¹ is O, n is 1, R₁ is hydrogen, and R₂ is hydrogen.

In some compounds, X¹ is O, n is 1, R₁ is hydrogen, R₂ is hydrogen, X is —C(R₃R₄)O—, R₃ is hydrogen, and R₄ is substituted alkyl.

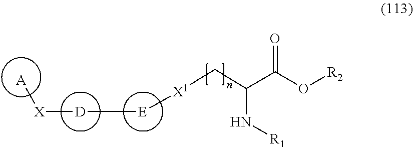

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; E is optionally substituted aryl or heterocycle; R₁ is H or alkyl; R₂ is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; n is 0-3; and X¹ is N, O, or S.

In some compounds, X¹ is O.

In some compounds, X¹ is O, n is 1, R₁ is hydrogen, and R₂ is hydrogen.

In some compounds, X¹ is O, n is 1, R₁ is hydrogen, R₂ is hydrogen, X is —C(R₃R₄)O—, R₃ is hydrogen, and R₄ is substituted alkyl. In some of these embodiments, A is fluoro-substituted biphenyl and R₃ is hydrogen. In some of these embodiments, A is 3′-fluorobiphenyl. In some of these embodiments, R₄ is halo-substituted methyl. In some of these embodiments, D is substituted pyrimidinyl and E is phenyl. In some of these embodiments, D is 2-substituted pyrimidinyl.

In some compounds, A is optionally substituted biphenyl, X is —C(R₃R₄)O—, R₃ is hydrogen, R₄ is optionally substituted lower alkyl, D is optionally substituted pyrimidinyl, E is phenyl, X¹ is O, n is 1, R₁ is hydrogen or lower alkyl, and R₂ is hydrogen or lower alkyl. In some of these compounds, A is 2′-, 3′-, 4′-, 5′, or 6′-halo-substituted biphenyl, X is —C(R₃R₄)O—, R₃ is hydrogen, R₄ is halo-substituted methyl or ethyl, D is 2-substituted pyrimidinyl, E is phenyl, X¹ is O, n is 1, R₁ is hydrogen, and R₂ is hydrogen. In some of these compounds, A is 2′-, 3′-, 4′-, 5′, or 6′-fluoro-substituted biphenyl, X is —C(R₃R₄)O—, R₃ is hydrogen, R₄ is fluoro-substituted methyl, D is 2-amino substituted pyrimidinyl, E is phenyl, X¹ is O, n is 1, R₁ is hydrogen, and R₂ is hydrogen.

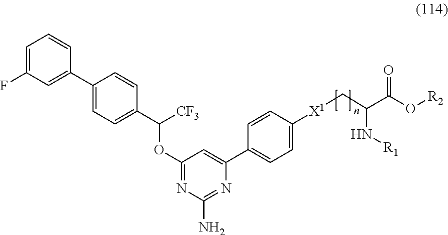

where:

### X¹ is N, O, or S;

R₁ is hydrogen or optionally substituted alkyl;
R₂ is hydrogen or optionally substituted alkyl, alkyl-aryl, alkyl-heterocycle, aryl, or heterocycle; and
n is 0-3.

In some compounds, X¹ is O and n is 1. In certain embodiments, the carbon having the HNR¹ group is in the S configuration.

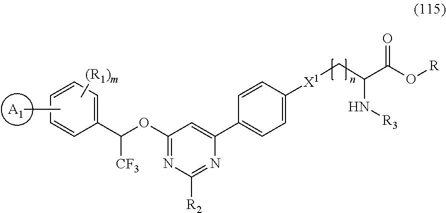

where A₁ is optionally substituted aryl or heterocycle; R is hydrogen or optionally substituted alkyl, alkyl-aryl, alkyl-heterocycle, aryl, or heterocycle; each R₁ is independently halogen, hydrogen, C(O)R_{A}, OR_{A}, NRₐR_{C}, S(O₂)R_{A}, or optionally substituted alkyl, alkyl-aryl or alkyl-heterocycle; R₂ is halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen or optionally substituted alkyl; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; X¹ is N, O, or S; m is 1-4; and n is 0-3. In certain embodiments, the carbon having the HNR₃ group is in the S configuration.

In some compounds, X¹ is O and n is 1.

In some compounds, X¹ is O, n is 1, R₁ is hydrogen, and R₂ is hydrogen.

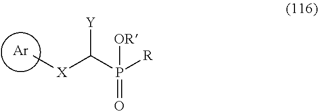

where:
Ar is a structure comprising multiple aryl or heterocycle rings;

### X is —CH₂— or N;

Y is hydrogen or NH₂;
R is optionally substituted alkyl or alkoxy; and
R′ is hydrogen or optionally substituted alkyl, alkyl-aryl, alkyl-heterocycle, aryl, or heterocycle.

In some compounds, X is N, Y is NH₂, R is optionally substituted alkyl, and R′ is hydrogen.

In some compounds, Ar is a structure comprising 1-4 optionally substituted linked cycloalkyl, aryl, or heterocycle rings. In some compounds, Ar comprises 1 ring; in some compounds, Ar comprises 2 rings; in some compounds, Ar comprises 3 rings; in some compounds, Ar comprises 4 rings.

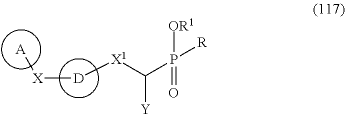

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; X¹ is —CH₂— or N; Y is hydrogen or NH₂; R is optionally substituted alkyl or alkoxy; and R¹ is hydrogen or optionally substituted alkyl, alkyl-aryl, alkyl-heterocycle, aryl, or heterocycle. In certain embodiments where Y is NH₂, the carbon to which Y is attached is in the S configuration. In other embodiments, the carbon to which Y is attached is in the R configuration.

In some compounds, X¹ is N, Y is NH₂, R is optionally substituted alkyl, and R′ is hydrogen.

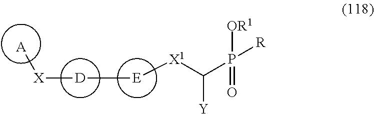

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; E is optionally substituted aryl or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; X¹ is —CH₂— or N; Y is hydrogen or NH₂; R is optionally substituted alkyl or alkoxy; and R¹ is hydrogen or optionally substituted alkyl, alkyl-aryl, alkyl-heterocycle, aryl, or heterocycle. In certain embodiments where Y is NH₂, the carbon to which Y is attached is in the S configuration. In other embodiments, the carbon to which Y is attached is in the R configuration.

In some compounds, X¹ is N, Y is NH₂, R is optionally substituted alkyl, and R′ is hydrogen.

In some compounds, A is optionally substituted biphenyl, X is —C(R₃R₄)O—, R₃ is hydrogen, R₄ is optionally substituted lower alkyl, D is optionally substituted pyrimidinyl, E is phenyl, X¹ is —CH₂— or N; Y is hydrogen or NH₂, R is optionally substituted alkyl, and R¹ is hydrogen or lower alkyl. In some of these compounds, A is 2′-, 3′-, 4′-, 5′, or 6′-halo-substituted biphenyl, X is —C(R₃R₄)O—, R₃ is hydrogen, R₄ is halo-substituted methyl or ethyl, D is 2-substituted pyrimidinyl, E is phenyl, X′ is N; Y is hydrogen, R is optionally substituted lower alkyl, and R¹ is hydrogen. In some of these compounds, A is 2′-, 3′-, 4′-, 5′, or 6′-fluoro-substituted biphenyl, X is —C(R₃R₄)O—, R₃ is hydrogen, R₄ is fluoro-substituted methyl, D is 2-amino substituted pyrimidinyl, E is phenyl, X¹ is N; Y is hydrogen, R is optionally substituted methyl or ethyl, and R¹ is hydrogen.

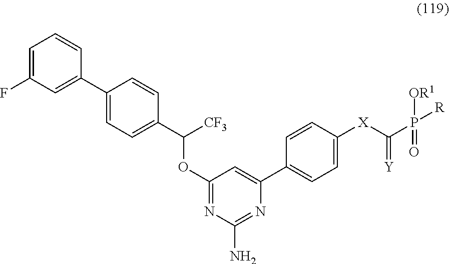

where:

### X is —CH₂— or N;

Y is hydrogen or NH₂;
R is hydrogen or optionally substituted alkyl or alkoxy; and
R′ is R is hydrogen or optionally substituted alkyl, alkyl-aryl, alkyl-heterocycle, aryl, or heterocycle. In certain embodiments where Y is NH₂, the carbon to which Y is attached is in the S configuration. In other embodiments, the carbon to which Y is attached is in the R configuration.

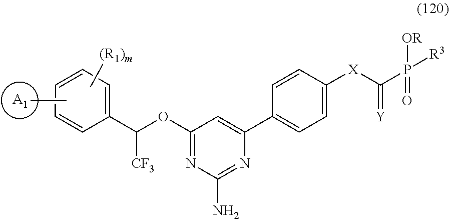

where A₁ is optionally substituted aryl or heterocycle; R is hydrogen or optionally substituted alkyl, alkyl-aryl, alkyl-heterocycle, aryl, or heterocycle; each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen or optionally substituted alkyl; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; X is —CH₂— or N; Y is hydrogen or NH₂; and m is 1-4. In certain embodiments where Y is NH₂, the carbon to which Y is attached is in the S configuration. In other embodiments, the carbon to which Y is attached is in the R configuration.

The present invention provides pharmaceutical compositions comprising a therapeutically effective amount of a TPH1 inhibitor disclosed herein and at least one pharmaceutically acceptable excipient. In certain embodiments, the TPH1 inhibitor may be in the form of a salt with a physiologically acceptable acid or base.

In certain embodiments, the patient's level of serum or plasma serotonin is measured prior to administering a therapeutic agent that lowers the level of serum or plasma serotonin. In other embodiments, the patient's level of serum or plasma serotonin is measured after administering the therapeutic agent that lowers the level of serum or plasma serotonin. In some embodiments, the patient's level of serum or plasma serotonin is measured before and after administering the therapeutic agent that lowers the level of serum or plasma serotonin.

In certain embodiments, the therapeutic agent that lowers the level of serum or plasma serotonin is repeatedly administered to the patient and the patient's level of serum or plasma serotonin is repeatedly measured until the patient's level of serum or plasma serotonin is reduced to a desired level, e.g., by at least about 10%, at least about 20%, at least about 30%, at least about 40%, at least about 50%, at least about 60%, at least about 70%, at least about 80%, or at least about 90%, compared to the level measured prior to the first administration of the therapeutic agent that lowers the level of serum or plasma serotonin.

In certain embodiments, the patient has been identified as having a serum or plasma serotonin level that is more than 10%, 25%, 35%, 50%, 75%, 100%, or 200% higher than the normal level of serum or plasma serotonin.

In certain embodiments, the patient's level of serum or plasma serotonin is lowered by at least about 10%, at least about 20%, at least about 30%, at least about 40%, at least about 50%, at least about 60%, at least about 70%, at least about 80%, or at least about 90% compared to the level before administering the therapeutic agent that lowers the level of serum or plasma serotonin.

In certain embodiments, the therapeutic agent that lowers the level of serum or plasma serotonin is administered in an amount of from about 1 mg/day to about 2 g/day.

The present invention provides a pharmaceutical composition comprising an amount of a therapeutic agent that lowers the level of serum or plasma serotonin in a patient to whom the composition is administered by at least about 10%, at least about 20%, at least about 30%, at least about 40%, at least about 50%, at least about 60%, at least about 70%, at least about 80%, or at least about 90%.

The present invention provides a pharmaceutical composition comprising a therapeutically effective amount of a therapeutic agent that lowers the level of serum or plasma serotonin in a patient to whom the pharmaceutical composition is administered, where the pharmaceutical composition also lowers blood levels of cholesterol, triglycerides, glycerol, and/or free fatty acids in a patient to whom the pharmaceutical composition is administered.

The present invention also provides a method for identifying a patient having hyperlipidemia or atherosclerosis or at risk of developing hyperlipidemia or atherosclerosis and treating the patient, comprising:

a) determining the level of serum or plasma serotonin in a biological sample taken from the patient and in a biological sample taken from a normal subject;

b) administering to the patient a therapeutically effective amount of a therapeutic agent disclosed herein if the level of serum or plasma serotonin in the sample from the patient is elevated by at least about 25% above the serum or plasma serotonin level in the sample from the normal subject;

whereby the patient's serum or plasma serotonin level is lowered and hyperlipidemia or atherosclerosis is thereby treated.

In certain embodiments, “determining the level of serum or plasma serotonin” of step (a) includes the formation of a complex between the serum or plasma serotonin from the biological sample and a reagent that specifically binds to serotonin.

In certain embodiments, “determining the level of serum or plasma serotonin” of step (a) includes a process whereby the serum or plasma serotonin is transformed into a derivative of serotonin, e.g., N-acylserotonin.

In certain embodiments, the “administering to the patient a therapeutically effective amount of a therapeutic agent” of step b) results in the lowering of blood levels of cholesterol, triglycerides, glycerol, and/or free fatty acids in the patient.

### BRIEF DESCRIPTION OF THE FIGURES

FIG. 1. Schematic of the design of the experiments described in the Examples herein.

FIG. 2. Administration of TPH1 inhibitor in ApoE−/− mice results in decreased atheroma formation. Left panel (FIG. 2A): representative picture of en face projection of Oil Red O stained (stained for neutral lipids) aortas from ApoE−/− mice treated with placebo or TPH1 inhibitor LP-533401 (atherosclerotic plaques seen as dark areas in the vessel). Middle panel (FIG. 2B): quantification of area occupied by atherosclerotic plaques; white bar—placebo control (n=6), black bar—TPH1 inhibitor treated animals (n=7). Right panel (FIG. 2C): serotonin plasma levels in ApoE−/− mice treated with placebo (white bar, n=6) or TPH1 inhibitor (black bar, n=7).

FIG. 3. Administration of TPH1 inhibitor in LDLR−/− mice lowers atheroma formation. Left panel (FIG. 3A): representative picture of en face projection of Oil Red O stained aortas from LDLR−/− mice treated with placebo or TPH1 inhibitor LP-533401 (atherosclerotic plaques seen as dark areas in the vessel). Middle panel (FIG. 3B): quantification of area occupied by atherosclerotic plaques; white bar—placebo control (n=6), black bar—TPH1 inhibitor treated mice (n=6). Right panel (FIG. 3C): serotonin plasma levels in LDLR−/− animals treated with placebo (white bar, n=6) or TPH1 inhibitor (black bar, n=6).

FIG. 4. TPH1 inhibitor attenuates cholesterol levels in ApoE−/− and LDLR−/− mice. Left panel (FIG. 4A): cholesterol levels in placebo (white bar, n=6) and TPH1 inhibitor treated (black bar, n=7) ApoE−/− mice fed a high cholesterol diet. Middle panel (FIG. 4B): plasma cholesterol levels in placebo (white bar, n=6) and TPH1 inhibitor treated (black bar, n=6) LDLR−/− mice fed a high cholesterol diet. Right panel (FIG. 4C): cholesterol levels in wild type mice treated with TPH1 inhibitor (black bar, n=5 and) and placebo treated control (white bar, n=5).

FIG. 5. TPH1 inhibitor does not alter triglyceride levels in ApoE−/− and LDLR−/− mice but lowers triglyceride levels in wild-type mice. Left panel (FIG. 5A): triglyceride levels in placebo (white bar, n=6) and TPH1 inhibitor treated (black bar, n=7) ApoE−/− mice fed a high cholesterol diet. Middle panel (FIG. 5B): levels of triglycerides in placebo (white bar, n=6) and TPH1 inhibitor treated (black bar, n=6) LDLR−/− mice fed a high cholesterol diet. Right panel (FIG. 5C): triglycerides levels in wild type mice treated with placebo (white bar, n=5) or TPH1 inhibitor (black bar, n=5).

FIG. 6. TPH1 inhibitor attenuates glycerol levels. Left panel (FIG. 6A): glycerol levels in placebo (white bar, n=6) and TPH1 inhibitor treated (black bar, n=7) ApoE−/− mice fed a high cholesterol diet. Middle panel (FIG. 6B): glycerol levels in placebo (white bar, n=5) and TPH1 inhibitor treated (black bar, n=5) ApoE−/− mice fed a rodent chow diet. Right panel (FIG. 6C): glycerol levels in placebo (white bar, n=6) and TPH1 inhibitor treated (black bar, n=6) wild type mice.

FIG. 7. TPH1 inhibitor attenuates free fatty acid (FFA) levels. Left panel (FIG. 7A): FFA levels in placebo (white bar, n=6) and TPH1 inhibitor treated (black bar, n=7) ApoE−/− mice fed a high cholesterol diet. Middle panel (FIG. 7B): FFA levels in placebo (white bar, n=5) and TPH1 inhibitor treated (black bar, n=5) ApoE−/− mice fed a rodent chow diet. Right panel (FIG. 7C): FFA levels in placebo (white bar, n=6) and TPH1 inhibitor treated (black bar, n=6) wild type mice.

FIG. 8. Serotonin promotes lipolysis in adipose tissue. Upper panel (FIG. 8A): rate of FFA release from adipose tissue explants upon stimulation with serotonin. Lower panel (FIG. 8B): glycerol release from adipose tissue explants upon stimulation with increasing concentrations of serotonin.

FIG. 9. Stimulation of the expression of lipolytic enzymes evoked by serotonin. Expression of mRNA of the indicated genes involved in the regulation of lipolysis in adipose tissue explants upon stimulation with 25 μM serotonin. Data are presented as relative expression levels compared to the untreated control (n=5 for each condition).

FIG. 10. Increase of blood serotonin levels in fasted mice. Mice were fasted for the indicated times. Data represent the average from 5 mice in each condition.

### DETAILED DESCRIPTION OF THE INVENTION

The term “substituted,” when used to describe a chemical structure or moiety, refers to a derivative of that structure or moiety wherein one or more of its hydrogen atoms is substituted with an atom, chemical moiety or functional group such as, but not limited to, alcohol, aldehyde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxygen (i.e., to provide an oxo group), phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

The term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

The term “alkyl” means a straight chain, branched and/or cyclic (“cycloalkyl”) hydrocarbon having from to 20 (e.g., 1 to 10 or 1 to 4) carbon atoms. Alkyl moieties having from 1 to 4 carbons are referred to as “lower alkyl.” Examples of alkyl groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

The term “alkoxy” means an —O-alkyl group. Examples of alkoxy groups include —OCH₃, —OCH₂CH₃, —O(CH₂)₂CH₃, —O(CH₂)₃CH₃, —O(CH₂)₄CH₃, and —O(CH₂)₅CH₃.

Term “alkylaryl” or “alkyl-aryl” means an alkyl moiety bound to an aryl moiety.

The term “alkylheteroaryl” or “alkyl-heteroaryl” means an alkyl moiety bound to a heteroaryl moiety.

The term “alkylheterocycle” or “alkyl-heterocycle” means an alkyl moiety bound to a heterocycle moiety.

The term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

The term “aryl” means an aromatic ring or an aromatic or partially aromatic ring system composed of carbon and hydrogen atoms. An aryl moiety may comprise multiple rings bound or fused together. Examples of aryl moieties include anthracenyl, azulenyl, biphenyl, fluorenyl, indan, indenyl, naphthyl, phenanthrenyl, phenyl, 1,2,3,4-tetrahydro-naphthalene, and tolyl.

The term “arylalkyl” or “aryl-alkyl” means an aryl moiety bound to an alkyl moiety.

The terms “halogen” and “halo” encompass fluorine, chlorine, bromine, and iodine.

The term “heteroalkyl” refers to an alkyl moiety (e.g., linear, branched or cyclic) in which at least one of its carbon atoms has been replaced with a heteroatom (e.g., N, O or S).

The term “heteroaryl” means an aryl moiety wherein at least one of its carbon atoms has been replaced with a heteroatom (e.g., N, O or S). Examples include acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

The term “heteroarylalkyl” or “heteroaryl-alkyl” means a heteroaryl moiety bound to an alkyl moiety.

The term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

The term “heterocyclealkyl” or “heterocycle-alkyl” refers to a heterocycle moiety bound to an alkyl moiety.

The term “heterocycloalkyl” refers to a non-aromatic heterocycle.

The term “heterocycloalkylalkyl” or “heterocycloalkyl-alkyl” refers to a heterocycloalkyl moiety bound to an alkyl moiety.

Prevention of or “preventing” hyperlipidemia or atherosclerosis means actively intervening as described herein prior to overt hyperlipidemia or atherosclerosis onset to prevent the development of hyperlipidemia or atherosclerosis or to minimize the extent of the hyperlipidemia or atherosclerosis or slow the course of development of the hyperlipidemia or atherosclerosis.

Treatment of or “treating” hyperlipidemia or atherosclerosis means actively intervening as described herein after the onset of hyperlipidemia or atherosclerosis to slow down, ameliorate symptoms of, minimize the extent of, or reverse the course of the hyperlipidemia or atherosclerosis in a patient who is known to have, is at risk of, or suspected to have, hyperlipidemia or atherosclerosis.

Unless otherwise indicated, a “therapeutically effective amount” of a therapeutic agent is an amount that provides a therapeutic benefit in the treatment or management of a disease or condition such as hyperlipidemia or atherosclerosis, delays or minimizes one or more symptoms associated with the disease or condition, or enhances the therapeutic efficacy of another therapeutic agent against the disease or condition. A therapeutic agent is said to be administered in a “therapeutically effective amount” if the amount administered results in a desired change in the physiology of a recipient mammal (e.g., decreasing the plasma levels of cholesterol, triglycerides, glycerol, and/or free fatty acids in a mammal having or at risk of developing hyperlipidemia or atherosclerosis) compared to pre-treatment levels. That is, the therapy results in treatment, i.e., modulates the recipient mammal's physiology to more closely resemble that of corresponding non-diseased state.

A “patient” is a mammal, preferably a human, but can also be a companion animal such as dogs or cats, or farm animals such as horses, cattle, pigs, or sheep.

In some embodiments, a patient “in need of prevention or treatment” for hyperlipidemia or atherosclerosis may include a patient known or suspected of having, or being at risk of developing, hyperlipidemia or atherosclerosis. Such a patient in need of treatment could be, e.g., a person known to have elevated levels of cholesterol, triglycerides, glycerol, and/or free fatty acids. Elevated levels of cholesterol may include blood levels of total cholesterol above 200 mg/dL, above 240 mg/dL, or above 300 mg/dL, or blood levels of low density lipoprotein cholesterol above 100 mg/dL, above 120 mg/dL, above 180 mg/dL, or above 220 mg/dL, when blood levels of total cholesterol or low density lipoprotein cholesterol are measured according to standard methods (e.g., under fasting conditions). Elevated levels of triglycerides may include blood levels above 150 mg/dL, above 200 mg/dL, or above 250 mg/dL, as measured under standard conditions, e.g., fasting conditions. Elevated levels of glycerol may include blood levels of above 1.5 mg/dL, above 2 mg/dL, above 5 mg/dL, above 7.5 mg/dL, or above 10 mg/dL, as measured under standard conditions, e.g., fasting conditions. Elevated levels of free fatty acids may include levels of above 200 μeq/L, above 300 μeq/L, above 400 μeq/L, or above 500 μeq/L, as measured under standard conditions, e.g., fasting conditions.

In some embodiments, a patient “in need of prevention or treatment” for hyperlipidemia or atherosclerosis may include a patient known or suspected of having, a ratio of total cholesterol divided by high density lipoprotein cholesterol of greater than 3.5:1, greater than 4:1, greater than 4.5:1, greater than 5:1, or greater than 5.5:1.

In some embodiments, a patient at risk of developing hyperlipidemia or atherosclerosis could include the elderly and the obese. Other persons in need of treatment or prevention by the methods of the present invention include persons who are known to be in need of therapy to decrease serum or plasma serotonin levels in order to treat or prevent hyperlipidemia or atherosclerosis. In some embodiments, such persons might include persons who have been identified as having a serum or plasma serotonin level that is about 25% or more above that of serum or plasma serotonin levels in normal subjects.

In some embodiments, a patient at risk of developing hyperlipidemia or atherosclerosis could include patients who have been diagnosed as having familial hypercholesterolemia.

In one embodiment, a patient in need of treatment or prevention for hyperlipidemia or atherosclerosis by the methods of the present invention does not include a patient being treated with a TPH1 inhibitor where the patient is being treated with the TPH1 inhibitor only for a purpose other than to treat hyperlipidemia or atherosclerosis. Thus, a patient in need of treatment or prevention for hyperlipidemia or atherosclerosis by the methods of the present invention does not include a patient being treated with a TPH1 inhibitor only for the purpose of treating chemotherapy-induced emesis, carcinoid syndrome, or gastrointestinal disorders such as irritable bowel syndrome.

A patient in need of treatment or prevention for hyperlipidemia or atherosclerosis by the methods of the present invention does not include a patient being treated with a TPH1 inhibitor only for the purpose of treating gastrointestinal diseases and certain other disorders. Examples of specific diseases and disorders include abdominal pain (e.g., associated with medullary carcinoma of the thyroid), anxiety, carcinoid syndrome, celiac disease, constipation (e.g., constipation having an iatrogenic cause, and idiopathic constipation), Crohn's disease, depression, diabetes, diarrhea (e.g., bile acid diarrhea, enterotoxin-induced secretory diarrhea, diarrhea having an iatrogenic cause, idiopathic diarrhea (e.g., idiopathic secretory diarrhea), and traveler's diarrhea), emesis, functional abdominal pain, functional anorectal disorders, functional bloating, functional dyspepsia, functional gallbladder disorders, irritable bowel syndrome (IBS; including IBD-d, IBS-c and IBS-a), lactose intolerance, MEN types I and II, nausea, Ogilvie's syndrome, Pancreatic Cholera Syndrome, pancreatic insufficiency, pheochromacytoma, scleroderma, somatization disorder, sphincter of Oddi disorders, ulcerative colitis, and Zollinger-Ellison Syndrome. A patient in need of treatment or prevention for hyperlipidemia or atherosclerosis by the methods of the present invention does not include a patient being treated with a TPH1 inhibitor only for the purpose of treating these diseases and disorders.

A patient in need of treatment or prevention for hyperlipidemia or atherosclerosis by the methods of the present invention also does not include a patient being treated with a TPH1 inhibitor only for the purpose of treating the following diseases and disorders: acute and chronic hypertension, chronic obstructive pulmonary disease (COPD), pulmonary embolism (e.g., bronchoconstriction and pulmonary hypertension following pulmonary embolism), pulmonary hypertension (e.g., pulmonary hypertension associated with portal hypertension), and radiation pneumonitis (including that giving rise to or contributing to pulmonary hypertension). Others include abdominal migraine, adult respiratory distress syndrome (ARDS), carcinoid crisis, CREST syndrome (calcinosis, Raynaud's phenomenon, esophageal dysfunction, sclerodactyl), telangiectasia), serotonin syndrome, and subarachnoid hemorrhage.

A “TPH1 inhibitor” is a substance that reduces the amount of 5-hydroxytryptophan produced from tryptophan by TPH1 in a suitable assay, as compared to the amount of 5-hydroxytryptophan produced from tryptophan by TPH1 in the assay in the absence of the substance. Preferably, the reduction in the amount of 5-hydroxytryptophan produced is at least about 10%, at least about 10%, at least about 20%, at least about 40%, at least about 60%, at least about 80%, or at least about 90%. Examples of assays for determining the level of TPH1 inhibition by a substance are described in U.S. Patent Application Publication US 2009/0029993.

### Methods of Diagnosis and Treatment

The results disclosed herein show that elevated serum or plasma serotonin increases plasma levels of cholesterol, triglycerides, glycerol, and free fatty acids, leading to hyperlipidemia and atherosclerosis. Thus, certain embodiments of the invention are directed to methods for diagnosing and treating persons at risk of developing hyperlipidemia or atherosclerosis and to methods for treating or preventing hyperlipidemia or atherosclerosis by administering therapeutic agents that decrease the level of serum or plasma serotonin and thus decrease the plasma levels of cholesterol, triglycerides, glycerol, and free fatty acids.

One embodiment of the invention is directed to a method for determining if a patient is at risk of developing hyperlipidemia or atherosclerosis by measuring the patient's level of serum or plasma serotonin and then administering a therapeutic agent disclosed herein that is a TPH1 inhibitor to the patient if the patient's level of serum or plasma serotonin is elevated so as to indicate that the patient is at risk of developing hyperlipidemia or atherosclerosis. In one embodiment, if the patient's level of serum or plasma serotonin is known to be significantly higher (e.g., more than about 25% higher, more than about 50% higher, more than about 75% higher, more than about 100% higher) than the level in a normal subject, then the patient is at risk of developing hyperlipidemia or atherosclerosis and one or more TPH1 inhibitors that reduce serotonin synthesis, and thus serum or plasma serotonin levels, is administered to reduce (and preferably normalize) serum or plasma serotonin levels, thereby preventing hyperlipidemia or atherosclerosis from developing or minimizing the extent or the detrimental effects of hyperlipidemia or atherosclerosis, should hyperlipidemia or atherosclerosis develop. Patient monitoring will determine if an abnormal serum or plasma serotonin level is chronic. If it is chronic, then the patient may need to continue treatment over a prolonged period (e.g., for one month, six months, one year, two years, three years, or many years) to normalize serum or plasma serotonin levels and/or maintain normal levels of serum or plasma serotonin.

When a patient's level of serum or plasma serotonin is compared to the level of serum or plasma serotonin in a normal subject, it should be understood that “normal subject” refers to a person who is matched to the patient in those characteristics that would be expected to affect serum or plasma serotonin levels, e.g., gender, age, general health, medications being taken, etc.

### Methods of Treatment and Prevention of Hyperlipidemia and Atherosclerosis

The present invention provides a method of preventing or treating hyperlipidemia or atherosclerosis in a patient known or suspected to be in need of such prevention or treatment comprising administering to the patient a therapeutically effective amount of a therapeutic agent that decreases serum or plasma serotonin levels. In certain embodiments, the method comprises administering to the patient therapeutically effective amounts of two or more therapeutic agents disclosed herein that decrease serum or plasma serotonin levels.

TPH1 inhibitors that may be used in certain of the methods of the present invention include the following, including any racemic mixtures and individual enantiomers, pharmaceutically acceptable salts or solvates thereof:

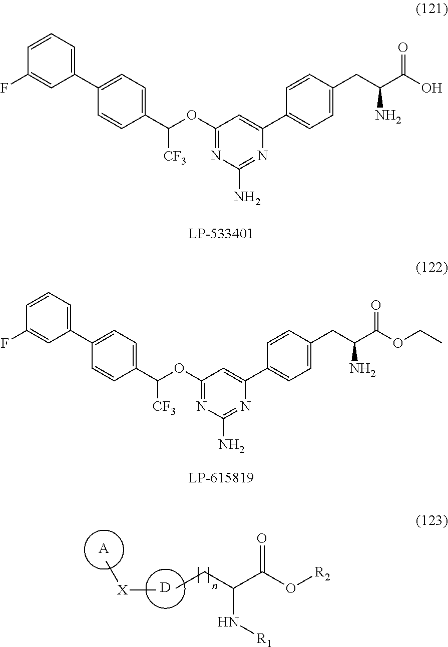

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or S(O₂)C(R₃R₄)—; D is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; and n is 0-3;

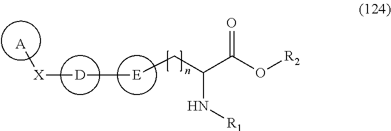

where A is optionally substituted cycloalkyl, aryl, or heterocycle; X is a bond (i.e., A is directly bound to D), —O—, —S—, —C(O)—, —C(R₄)═, ═C(R₄)—, —C(R₃R₄)—, —C(R₄)═C(R₄)—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; E is optionally substituted aryl or heterocycle; R₁ is hydrogen or optionally substituted alkyl, alkyl-aryl, alkyl-heterocycle, aryl, or heterocycle; R₂ is hydrogen or optionally substituted alkyl, alkyl-aryl, alkyl-heterocycle, aryl, or heterocycle; R₃ is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; and n is 0-3;

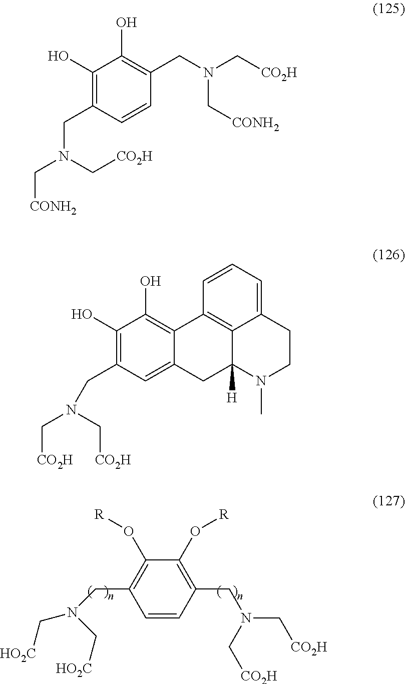

where R is hydrogen or lower alkyl; and
n is 1, 2, or 3;

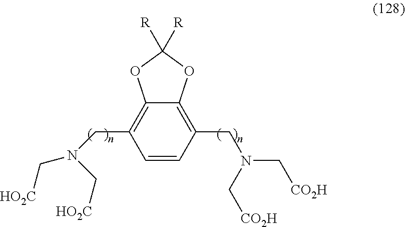

where R is hydrogen or lower alkyl; and
n is 1, 2, or 3;

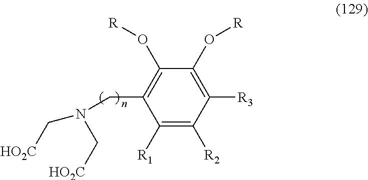

where R is hydrogen or lower alkyl;
R₁, R₂, and R₃, are independently:
hydrogen;
halogen;
lower alkyl;
alkoxy; or
amino; and
   n is 1, 2, or 3;

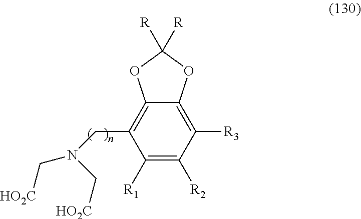

where R is hydrogen or lower alkyl;
R₁, R₂, and R₃, are independently:
hydrogen;
halogen;
lower alkyl;
alkoxy; or
amino; and
   n is 1, 2, or 3;

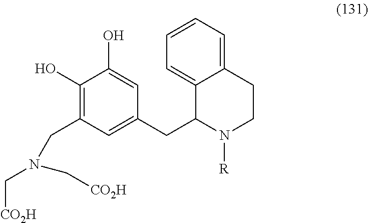

where R is hydrogen, lower alkyl, or cycloalkyl;

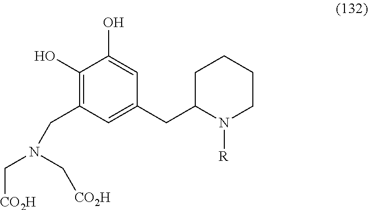

where R is hydrogen, lower alkyl, or cycloalkyl;

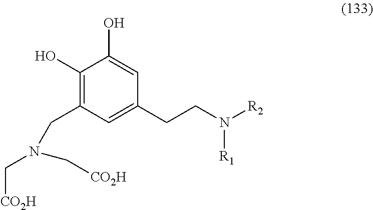

where R₁ and R₂ are independently hydrogen, lower alkyl, or cycloalkyl;

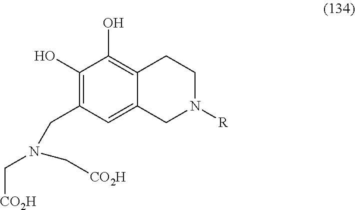

where R is hydrogen, lower alkyl, or cycloalkyl;

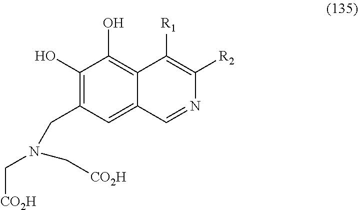

where R₁ and R₂ are independently hydrogen, lower alkyl, cycloalkyl, F, Cl, or OH;

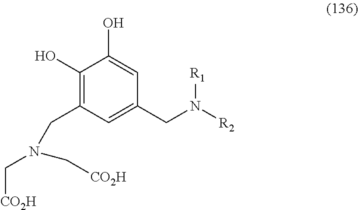

where R₁ and R₂ are independently hydrogen, lower alkyl, or cycloalkyl.

Other TPH1 inhibitors that may be used in the methods of the present invention include the following, including any racemic mixtures and individual enantiomers, pharmaceutically acceptable salts or solvates thereof:

[symbol]

### (137) (S)-2-Amino-3-(4-(4-amino-6-((R)-1-(naphthalen-2-yl)ethylamino)-1,3,5-triazin-2-yl)phenyl)propanoic acid;

### (138) (S)-2-Amino-3-(4-(4-amino-6-((4′-methylbiphenyl-4-yl)methylamino-1,3,5-triazin-2-yl)phenyl)propanoic acid;

### (139) (S)-2-Amino-3-(4-(4-morpholino-6-(naphthalen-2-ylmethylamino)-1,3,5-triazin-2-yl)phenyl)propanoic acid;

### (140) (2S)-2-Amino-3-(4-(2-amino-6-(2,2,2-trifluoro-1-(2-(trifluoromethyl)phenyl)ethoxy)pyrimidin-4-yl)phenyl)propanoic acid;

### (141) (2S)-2-Amino-3-(4-(2-amino-6-(2,2,2-trifluoro-1-p-tolylethoxy)pyrimidin-4-yl)phenyl)propanoic acid;

### (142) (2S)-2-Amino-3-(4-(2-amino-6-(1-cyclohexyl-2,2,2-trifluoroethoxy)pyrimidin-4-yl(phenyl)propanoic acid;

### (143) (S)-2-Amino-3-(4-(6-(2-fluorophenoxy)pyrimidin-4-yl)phenyl)propanoic acid;

### (144) (2S)-2-Amino-3-(4-(4-(3-(4-chlorophenyl)piperidin-1-yl)-1,3,5-triazin-2-yl)phenyl)propanoic acid;

### (145) (2S)-2-Amino-3-(4-(4-amino-6-(2,2,2-trifluoro-1-phenylethoxy)-1,3,5-triazin-2-yl)phenyl)propanoic acid;

### (146) (S)-2-Amino-3-(5-(4-amino-6-((R)-(naphthalen-2-yl)ethylamino)-1,3,5-triazin-2-yl)pyridin-2-yl)propanoic acid;

### (147) (S)-2-Amino-3-(3-(4-amino-6-(R)-1-(naphthalen-2-yl)ethylamino)-1,3,5-triazin-2-yl)-1H-pyrazol-1-yl)propanoic acid;

### (148) (S)-2-Amino-3-(4′-(3-(cyclopentyloxy)-4-methoxybenzylamino)biphenyl-4-yl)propanoic acid;

### (149) (S)-2-Amino-3-(4-(6-(3-(cyclopentyloxy)-4-methoxybenzylamino)pyrimidin-4-yl)phenyl)propanoic acid;

### (150) (S)-2-Amino-3-(4-(6-(3-(cyclopentyloxy)-4-methoxybenzylamino)pyrazin-2-yl)phenyl)propanoic acid;

### (151) (S)-2-Amino-3-(4-(5-((4′-methylbiphenyl-2-yl)methylamino)pyrazin-2-yl)phenyl)propanoic acid;

### (152) (2S)-2-Amino-3-(4-(6-(2,2,2-trifluoro-1-phenylethoxy)-pyrimidin-4-yl)phenyl)propanoic acid;

### (153) (2S)-2-Amino-3-(4-(6-(1-(3,4-difluorophenyl)-2,2,2-trifluoroethoxy)pyriinidin-4-yl)phenyl)propanoic acid;

### (154) (S)-2-Amino-3-(4-(5-(3-(cyclopentyloxy)-4-methoxybenzylamino)-pyrazin-2-yl)phenyl)propanoic acid;

### (155) (S)-2-Amino-3-(4-(5-((3-(cyclopentyloxy)-4-methoxybenzyl)-(methyl)amino)pyrazin-2-yl)phenyl)propanoic acid;

### (156) (S)-2-Amino-3-(4-(5-((1,3-dimethyl-1H-pyrazol-4-yl)methylamino)pyrazin-2-yl)phenyl)propanoic acid;

### (157) (S)-2-Amino-3-(4-(4-amino-6-((S)-1-(naphthalen-2-yl)ethylamino)-1,3,5-triazin-2-yloxy)phenyl)propanoic acid;

### (158) (S)-2-Amino-3-(4-(4-amino-6-((R)-1-(biphenyl-2-yl)-2,2,2-trifluoroethoxy)-1,3,5-triazin-2-yl)phenyl)propanoic acid;

### (159) (2S)-2-Amino-3-(4-(4-amino-6-(1-(6,8-difluoronaphthalen-2-yl)ethylamino)-1,3,5-triazin-2-yl)phenyl)propanoic acid;

### (160) (2S)-2-Amino-3-(4-(4-amino-6-(2,2,2-trifluoro-1-(3′-methylbiphenyl-2-yl)ethoxy)-1,3,5-triazin-2-yl)phenyl)propanoic acid;

### (161) (S)-2-Amino-3-(4-(5-(3,4-dimethoxyphenylcarbamoyl)-pyrazin-2-yl)phenyl)propanoic acid;

### (162) (S)-2-Amino-3-(4-(2-amino-6-(4-(2-(trifluoromethyl)phenyl)-piperidin-1-yl)pyrimidin-4-yl)phenyl)propanoic acid;

### (163) (S)-2-Amino-3-(4-(2-amino-6-((R)-1-(naphthalen-2-yl)ethylamino)pyrimidin-4-yl)phenyl)propanoic acid;

### (164) (S)-2-Amino-3-(4-(2-amino-6-(methyl(R)-1-(naphthalen-2-yl)ethyl)amino)pyrimidin-4-yl)phenyl)propanoic acid;

### (165) (S)-2-Amino-3-(4-(2-amino-6-((S)-2,2,2-trifluoro-1-(6-methoxynaphthalen-2-yl)ethoxy)pyrimidin-4-yl)phenyl)propanoic acid;

### (166) (S)-2-Amino-3-(4-(5-(biphenyl-4-ylmethylamino)pyrazin-2-yl)phenyl)propanoic acid;

### (167) (S)-2-Amino-3-(4-(5-(naphthalen-2-ylmethylamino)pyrazin-2-yl)phenyl)propanoic acid;

### (168) (S)-2-(Tert-butoxycarbonylamino)-3-(4-(5-(naphthalen-2-ylmethylamino)pyrazin-2-yl)phenyl)propanoic acid;

### (169) (S)-2-Morpholinoethyl 2-amino-3-(4-(5-(naphthalen-2-ylmethylamino)pyrazin-2-yl)phenyl)propanoate;

### (170) (S)-2-Amino-3-(4-(2-amino-6-(2,2,2-trifluoro-1-(3′-fluorobiphenyl-4-yl)ethoxy)pyrimidin-4-yl)phenyl)propanoic acid;

### (171) (S)-2-Amino-3-(4-(2-amino-6-(benzylthio)pyrimidin-4-yl)phenyl)propanoic acid;

### (172) (S)-2-Amino-3-(4-(2-amino-6-(naphthalen-2-ylmethylthio)pyrimidin-4-yl)phenyl)propanoic acid;

### (173) (2S)-2-Amino-3-(4-(2-amino-6-(1-(3,4-difluorophenyl)-2,2,2-trifluoroethoxy)pyrimidin-4-yl)phenyl)propanoic acid;

### (174) (2S)-2-Amino-3-(4-(2-amino-6-(2,2,2-trifluoro-1-(3′-methylbiphenyl-2-yl)ethoxy)pyrimidin-4-yl)phenyl)propanoic acid;

### (175) (S)-2-Amino-3-(4-(5-(3-(cyclopentyloxy)-4-methoxybenzylamino)pyridin-3-yl)phenyl)propanoic acid;

### (176) 2-Amino-3-(3-(4-amino-6-((R)-1-(naphthalen-2-yl)ethylamino)-1,3,5-triazin-2-yl)phenyl)propanoic acid;

### (177) 2-Amino-3-(4-(4-amino-6-((R)-1-(naphthalen-2-yl)ethylamino)-1,3,5-triazin-2-yl)-2-fluorophenyl)propanoic acid;

### (178) (2S)-2-Amino-3-(4-(4-amino-6-(1-(adamantyl)ethylamino)-1,3,5-triazin-2-yl)phenyl)propanoic acid;

### (179) (S)-2-Amino-3-(4-(5-fluoro-4-((R)-1-(naphthalen-2-yl)ethylamino)pyrimidin-2-yl)phenyl)propanoic acid;

### (180) (S)-2-Amino-3-(4-(2-amino-6-(4-(trifluromethyl)-benzylamino)pyrimidin-4-yl)phenyl)propanoic acid;

### (181) 2-Amino-3-(5-(5-phenylthiophen-2-yl)-1H-indol-3-yl)propanoic acid;

### (182) (S)-2-Amino-3-(4-(4-(4-phenoxyphenyl)-1H-1,2,3-triazol-1-yl)phenyl)propanoic acid;

### (183) (S)-2-Amino-3-(4-(4-(4-(thiophene-2-carboxamido)phenyl)-1H-1,2,3-triazol-1-yl)phenyl)propanoic acid; and

### (184) (S)-2-Amino-3-(4-(2-amino-6-(phenylethynyl)pyrimidin-4-yl)phenyl)propanoic acid;

Additional TPH1 inhibitors that may be used in the present invention include:

[symbol]

### (185) N-[(1R,4R,9aS)-4-phenyl octahydropyrido[2,1-c][1,4]oxazin-1-yl]3,4,5-trimethoxybenzamide;

### (186) 2,6-Piperidinedione, 3-[3-(dimethylamino)propyl]-3-(3-methoxyphenyl)-4,4-dimethyl-, monohydrochloride;

### (187)

Triptosine (CAS registry number 86248-47-7; U.S. Pat. No. 4,472,387);

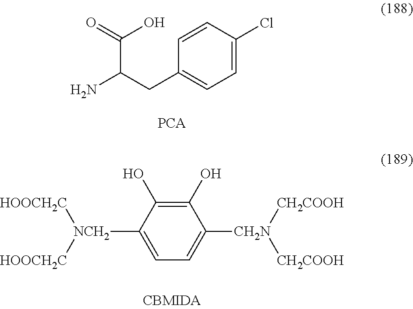

Additional TPH1 inhibitors that may be used in the present invention are listed in the table below.

TABLE 1
(S)-2-amino-3-(4-(5-(2-fluoro-4,5-
dimethoxybenzylamino)pyrazin-2-yl)phenyl)propanoic acid
(S)-2-amino-3-(4-(2-amino-6-(4-(2-methoxyphenyl)piperidin-1-
yl)pyrimidin-4-yl)phenyl)propanoic acid
(S)-2-amino-3-(4-(6-(3-(cyclopentyloxy)-4-
methoxybenzylamino)-2-(dimethylamino)pyrimidin-4-
yl)phenyl)propanoic acid
(S)-2-amino-3-(4-(5-(3,4-dimethylbenzylamino)pyrazin-2-
yl)phenyl)propanoic acid
(S)-2-amino-3-(4-(5-(biphenyl-2-ylmethylamino)pyrazin-2-
yl)phenyl)propanoic acid
(S)-ethyl 2-amino-3-(4-(2-amino-6-(4-
(trifluoromethyl)benzylamino)pyrimidin-4-yl)phenyl)propanoate
(S)-2-amino-3-(4-(5-(cyclopentylmethylamino)pyrazin-2-
yl)phenyl)propanoic acid
(2S)-2-amino-3-(4-(2-amino-6-(3-(2-
(trifluoromethyl)phenyl)pyrrolidin-l-yl)pyrimidin-4-
yl)phenyl)propanoic acid
(2S)-2-amino-3-(4-(2-amino-6-(1,2,3,4-tetrahydronaphthalen-1-
ylamino)pyrimidin-4-yl)phenyl)propanoic acid
(S)-2-amino-3-(4-(2-amino-6-((R)-1-(naphthalen-2-
yl)ethoxy)pyrimidin-4-yl)phenyl)propanoic acid
(2S)-2-amino-3-(4-(2-amino-6-(1,2-
diphenylethylamino)pyrimidin-4-yl)phenyl)propanoic acid
(S)-2- amino-3-(4-(2-amino-6-((R)-1-(4-(benzo[b]thiophen-3-
yl)phenyl)ethylamino)pyrimidin-4-yl)phenyl)propanoic acid
(S)-2-amino-3-(4-(4-amino-6-((R)-1-(4′-methoxybiphenyl-4-
yl)ethylamino)-1,3,5-triazin-2-yl)phenyl)propanoic acid
2-amino-3-(1-(4-amino-6-((R)-1-(naphthalen-2-yl)ethylamino)-
1,3,5-triazin-2-yl)piperidin-4-yl)propanoic acid
(2S)-2-amino-3-(4-(4-amino-6-(1-(4-fluoronaphthalen-1-
yl)ethylamino)-1,3,5-triazin-2-yl)phenyl)propanoic acid
(S)-2-amino-3-(4-(4-amino-6-((3′-fluorobiphenyl-4-
yl)methylamino)-1,3,5-triazin-2-yl)phenyl)propanoic acid
2-amino-3-(4-(4-amino-6-((R)-1-(naphthalen-2-yl)ethylamino)-
1,3,5-triazin-2-yl)-2-fluorophenyl)propanoic acid
(S)-2-amino-3-(4-(2-amino-6-((R)-2,2,2-trifluoro-1-(3′-
methoxybiphenyl-4-yl)ethoxy)pyrimidin-4-yl)phenyl)propanoic
acid
(2S)-2-amino-3-(4-(4-amino-6-(2,2,2-trifluoro-1-(3′-
fluorobiphenyl-2-yl)ethoxy)-1,3,5-triazin-2-yl)phenyl)propanoic
acid
(2S)-2-amino-3-(4-(4-amino-6-(1-(4-tert-
butylphenyl)ethylamino)-1,3,5-triazin-2-yl)phenyl)propanoic
acid
(2S)-2-amino-3-(4-(2-amino-6-(2,2,2-trifluoro-1-(3′-
fluorobiphenyl-4-yl)ethoxy)pyrimidin-4-yl)phenyl)propanoic
acid
(2S)-2-amino-3-(4-(4-amino-6-(6,7-dihydroxy-1-methy1-3,4-
dihydroisoquinolin-2(1H)-yl)-1,3,5-triazin-2-
yl)phenyl)propanoic acid
(2S)-2-amino-3-(4-(4-amino-6-(2,2,2-trifluoro-1-(3′-
methylbiphenyl-4-yl)ethoxy)-1,3,5-triazin-2-
yl)phenyl)propanoic acid
(S)-2-amino-3-(4-(4-amino-6-((R)-1-(naphthalen-2-
yl)ethylamino)pyrimidin-2-yl)phenyl)propanoic acid
(S)-2-amino-3-(4-(2-amino-6-(benzylthio)pyrimidin-4-
yl)phenyl)propanoic acid
(2S)-2-amino-3-(4-(2-amino-6-(2,2,2-trifluoro-1-(4′-
fluorobiphenyl-4-yl)ethoxy)pyrimidin-4-yl)phenyl)propanoic
acid
(2S)-2-amino-3-(4-(6-(3-(4-chlorophenoxy)piperidin-1-
yl)pyrimidin-4-yl)phenyl)propanoic acid
(S)-3-(4-(4-amino-6-((R)-1-(naphthalen-2-yl)ethylamino)-1,3,5-
triazin-2-yl)pheny1)-2-(2-aminoacetamido)propanoic acid
(S)-2-amino-3-(4-(6-((R)-1-(naphthalen-2-yl)ethylamino)-2-
(trifluoromethyl)pyrimidin-4-yl)phenyl)propanoic acid
(S)-2-amino-3-(4-(2-amino-6-(4-(3-chlorophenyl)piperazin-l-
yl)pyrimidin-4-yl)phenyl)propanoic acid
(S)-2-amino-3-(4-(2-amino-6-((R)-2,2,2-trifluoro-1-
phenylethoxy)pyrimidin-4-y1)phenyl)propanoic acid
(2S)-2-amino-3-(4-(2-amino-6-(1,4-
diphenylbutylamino)pyrimidin-4-yl)phenyl)propanoic acid
(2S)-2-amino-3-(4-(6-(1-(3′-chlorobipheny1-2-yl)-2,2,2-
trifluoroethoxy)pyrimidin-4-yl)phenyl)propanoic acid
(2S)-2-amino-3-(4-(4-amino-6-(1-(biphenyl-4-yl)-2,2,2-
trifluoroethoxy)-1,3,5-triazin-2-yl)phenyl)propanoic acid
(2S)-2-amino-3-(4-(2-amino-6-(2,2,3,3,3-pentafluoro-1-(3-
fluoro-4-methylphenyl)propoxy)pyrimidin-4-
yl)phenyl)propanoic acid
(S)-ethyl 2-amino-3-(4-(2-amino-6-((R)-2,2,2-trifluoro-1-(3′-
methoxybiphenyl-4-yl)ethoxy)pyrimidin-4-
yl)phenyl)propanoate
(S)-2-amino-3-(4-(2-amino-6-((S)-2,2,2-trifluoro-1-(3′-
methoxybiphenyl-4-yl)ethoxy)pyrimidin-4-yl)phenyl)propanoic
acid
(2S)-2-amino-3-(4-(2-amino-6-(2,2,2-trifluoro-1-(3-fluoro-3′-
methoxybiphenyl-4-yl)ethoxy)pyrimidin-4-yl)phenyl)propanoic
acid
(2S)-2-amino-3-(4-(2-amino-6-(1-(3′-(dimethylamino)biphenyl-
2-y1)-2,2,2-trifluoroethoxy)pyrimidin-4-yl)phenyl)propanoic
acid
(2S)-2-amino-3-(4-(2-amino-6-(2,2,2-trifluoro-1-(3′-methoxy-5-
methylbiphenyl-2-yl)ethoxy)pyrimidin-4-yl)phenyl)propanoic
acid
(2S)-2-amino-3-(4-(2-amino-6-(2,2,2-trifluoro-1-(4′-methoxy-5-
methylbiphenyl-2-yl)ethoxy)pyrimidin-4-yl)phenyl)propanoic
acid
(2S)-2-amino-3-(4-(2-amino-6-(2,2,2-trifluoro-1-(3′-methoxy-3-
(methylsulfonyl)biphenyl-4-yl)ethoxy)pyrimidin-4-
yl)phenyl)propanoic acid
(2S)-2-amino-3-(4-(2-amino-6-(1-(2-(cyclopropylmethoxy)-4-
fluorophenyl)-2,2,2-trifluoroethoxy)pyrimidin-4-
yl)phenyl)propanoic acid
(2S)-2-amino-3-(4-(6-(1-(2-(cyclopropylmethoxy)-4-
fluorophenyl)-2,2,2-trifluoroethoxy)pyrimidin-4-
yl)phenyl)propanoic acid
(2S)-2-amino-3-(4-(2-amino-6-(2,2,2-trifluoro-1-(2-
(isopentyloxy)phenyl)ethoxy)pyrimidin-4-yl)phenyl)propanoic
acid
(2S)-2-amino-3-(4-(5-(2,2,2-trifluoro-1-(3′-fluorobiphenyl-4-
yl)ethoxy)pyrazin-2-yl)phenyl)propanoic acid
(2S)-2-amino-3-(4-(2-amino-6-(2,2,2-trifluoro-1-(4′-
methoxybiphenyl-2-yl)ethoxy)pyrimidin-4-yl)phenyl)propanoic
acid
(2S)-2-amino-3-(4-(2-amino-6-(1-(3′-carbamoylbiphenyl-2-yl)-
2,2,2-trifluoroethoxy)pyrimidin-4-yl)phenyl)propanoic acid
(2S)-2-amino-3-(4-(2-amino-6-(1-(4′-carbamoylbiphenyl-2-yl)-
2,2,2-trifluoroethoxy)pyrimidin-4-yl)phenyl)propanoic acid
(2S)-2-amino-3-(4-(2-amino-6-(2,2,2-trifluoro-1-(4-(2-
methoxyphenoxy)phenyl)ethoxy)pyrimidin-4-
yl)phenyl)propanoic acid
(2S)-2-amino-3-(4-(6-(2,2,2-trifluoro-1-(4-(2-
methoxyphenoxy)phenyl)ethoxy)pyrimidin-4-
yl)phenyl)propanoic acid
(2S)-2-amino-3-(4-(6-(2,2,2-trifluoro-1-(2-
(isopentyloxy)phenyl)ethoxy)pyrimidin-4-yl)phenyl)propanoic
acid
(2S)-3-(4-(6-(1-(3′-acetamidobiphenyl-2-yl)-2,2,2-
trifluoroethoxy)-2-aminopyrimidin-4-yl)phenyl)-2-
aminopropanoic acid
(2S)-3-(4-(6-(1-(4′-acetamidobiphenyl-2-yl)-2,2,2-
trifluoroethoxy)-2-aminopyrimidin-4-yl)phenyl)-2-
aminopropanoic acid
(2S)-2-amino-3-(4-(2-amino-6-(1-(4-cyanophenyl)-2,2,2-
trifluoroethoxy)pyrimidin-4-yl)phenyl)propanoic acid
(S)-ethyl 2-amino-3-(4-(2-amino-6-((R)-2,2,2-trifluoro-l-p-
tolylethoxy)pyrimidin-4-yl)phenyl)propanoate
(2S)-2-amino-3-(4-(2-amino-6-(2,2,2-trifluoro-1-(1-
methoxybicyclo[2.2.2]oct-5-en-2-yl)ethoxy)pyrimidin-4-
yl)phenyl)propanoic acid
(2S)-2-amino-3-(4-(2-amino-6-(1-(4-(cyclopentyloxy)phenyl)-
2,2,2-trifluoroethoxy)pyrimidin-4-yl)phenyl)propanoic acid
(2S)-2-amino-3-(4-(6-(1-(4-(cyclopentyloxy)phenyl)-2,2,2-
trifluoroethoxy)pyrimidin-4-yl)phenyl)propanoic acid
(2S)-2-amino-3-(4-(2-amino-6-(2,2,2-trifluoro-1-(4-(3-
methoxyphenoxy)phenyl)ethoxy)pyrimidin-4-
yl)phenyl)propanoic acid
(2S)-2-amino-3-(4-(2-amino-6-(1-(4,5-dimethoxyhiphenyl-2-yl)-
2,2,2-trifluoroethoxy)pyrimidin-4-yl)phenyl)propanoic acid
(2S)-2-amino-3-(4-(2-amino-6-(1-(4,5-dimethoxy-3′-
methylbiphenyl-2-yl)-2,2,2-trifluoroethoxy)pyrimidin-4-
yl)phenyl)propanoic acid
(2S)-2-amino-3-(4-(5-(2,2,2-trifluoro-1-(2′-methylbiphenyl-2-
yl)ethoxy)pyrazin-2-yl)phenyl)propanoic acid
(2S)-2-amino-3-(4-(6-(2,2,2-trifluoro-1-(4-(3-
methoxyphenoxy)phenyl)ethoxy)pyrimidin-4-
yl)phenyl)propanoic acid
(2S)-2-amino-3-(4-(2-amino-6-(1-(2-(3,5-
difluorophenoxy)phenyl)-2,2,2-trifluoroethoxy)pyrimidin-4-
yl)phenyl)propanoic acid
(2S)-2-amino-3-(4-(2-amino-6-(2,2,2-trifluoro-1-(4-(4-
methoxyphenoxy)phenyl)ethoxy)pyrimidin-4-
yl)phenyl)propanoic acid
(2S)-2-amino-3-(4-(2-amino-6-(1-(4′-((S)-2-amino-2-
carboxyethyl)biphenyl-2-yl)-2,2,2-trifluoroethoxy)pyrimidin-4-
yl)phenyl)propanoic acid
(2S)-2-amino-3-(4-(2-arnino-6-(1-(2-bromophenyl)-2,2,2-
trifluoroethoxy)pyrimidin-4-yl)phenyl)propanoic acid
(2S)-2-amino-3-(4-(5-(2,2,2-trifluoro-1-(3′-methylbiphenyl-2-
yl)ethoxy)pyrazin-2-yl)phenyl)propanoic acid
(2S)-2-amino-3-(4-(2-amino-6-(2,2,2-trifluoro-1-(4-
methoxybiphenyl-2-yl)ethoxy)pyrimidin-4-yl)phenyl)propanoic
acid
(2S)-2-amino-3-(4-(5-(2,2,2-trifluoro-1-(2-(4-methylthiophen-3-
yl)phenyl)ethoxy)pyrazin-2-yl)phenyl)propanoic acid
(2S)-2-amino-3-(4-(2-amino-6-(2,2,2-trifluoro-1-(4-methoxy-3′-
methylbiphenyl-2-yl)ethoxy)pyrimidin-4-yl)phenyl)propanoic
acid
(2S)-2-amino-3-(4-(2-amino-6-(2,2,2-trifluoro-1-(3′-
(hydroxymethyl)biphenyl-2-yl)ethoxy)pyrimidin-4-
yl)phenyl)propanoic acid
(2S)-2-amino-3-(4-(2-amino-6-(1-(3′-cyanobiphenyl-2-yl)-2,2,2-
trifluoroethoxy)pyrimidin-4-yl)phenyl)propanoic acid
(2S)-2-amino-3-(4-(6-(1-(2-(3,5-difluorophenoxy)phenyl)-2,2,2-
trifluoroethoxy)pyrimidin-4-yl)phenyl)propanoic acid
(2S)-2-amino-3-(4-(6-(2,2,2-trifluoro-1-(4-(4-
methoxyphenoxy)phenyl)ethoxy)pyrimidin-4-
yl)phenyl)propanoic acid
(2S)-2-amino-3-(4-(2-amino-6-(2,2,2-trifluoro-1-(2-(4-
methylthiazol-2-yl)thiophen-3-yl)ethoxy)pyrimidin-4-
yl)phenyl)propanoic acid
(2S)-2-amino-3-(4-(2-amino-6-(2,2,2-trifluoro-1-(5-(4-
methoxyphenyl)isoxazol-3-yl)ethoxy)pyrimidin-4-
yl)phenyl)propanoic acid
(2S)-2-amino-3-(4-(2-amino-6-(2,2,2-trifluoro-1-(1-phenyl-5-
(trifluoromethyl)-1H-pyrazol-4-yl)ethoxy)pyrimidin-4-
yl)phenyl)propanoic acid
(2S)-2-amino-3-(4-(2-amino-6-(1-(2-(cyclohexyloxy)-4-
methylphenyl)-2,2,2-trifluoroethoxy)pyrimidin-4-
yl)phenyl)propanoic acid
(2S)-2-amino-3-(4-(2-amino-6-(1-(2-(cyclopentyloxy)-4-
methylphenyl)-2,2,2-trifluoroethoxy)pyrimidin-4-
yl)phenyl)propanoic acid
(2S)-2-amino-3-(4-(2-amino-6-(1-(benzo[d]thiazol-6-yl)-2,2,2-
trifluoroethoxy)pyrimidin-4-yl)phenyl)propanoic acid
(2S)-2-amino-3-(4-(2-amino-6-(2,2,2-trifluoro-1-(1-methyl-1H-
imidazol-5-yl)ethoxy)pyrimidin-4-yl)phenyl)proparioic acid
(2S)-2-amino-3-(4-(6-(1-(2-(cyclopentyloxy)-4-methylphenyl)-
2,2,2-trifluorethoxy)pyrimidin-4-yl)phenyl)propanoic acid
(2S)-2-amino-3-(4-(6-(1-(2-(cyclohexyloxy)-4-methylphenyl)-
2,2,2-trifluoroethoxy)pyrimidin-4-yl)phenyl)propanoic acid
(2S)-2-amino-3-(4-(2-amino-6-(2,2,2-trifluoro-1-(pyridin-3-
yl)ethoxy)pyrimidin-4-yl)phenyl)propanoic acid
(2S)-2-amino-3-(4-(2-amino-6-(1-(1,3-dimethyl-1H-pyrazol-5-
y1)-2,2,2-trifluoroethoxy)pyrimidin-4-yl)phenyl)propanoic acid
(S)-2-amino-3-(4-(2-amino-6-(3-hydroxyphenyl)pyrimidin-4-
yl)phenyl)propanoic acid
(2S)-2-amino-3 -(4-(2-amino-6-(2,2,2-trifluoro-1-(3′-
hydroxybiphenyl-2-yl)ethoxy)pyrimidin-4-yl)phenyl)propanoic
acid
(S)-2-amino-3-(4-(2-amino-6-(3,5-difluorophenyl)pyrimidin-4-
yl)phenyl)propanoic acid
(2S)-2-amino-3-(4-(2-amino-6-(1-(3′,5′-difluorobiphenyl-2-yl)-
2,2,2-trifluoroethoxy)pyrimidin-4-yl)phenyl)propanoic acid
(2S)-2-amino-3-(4-(6-(2,2,2-trifluoro-1-(3′-fluorobiphenyl-3-
yl)ethoxy)pyrazin-2-yl)phenyl)propanoic acid
(2S)-2-amino-3-(4-(2-amino-6-(1-(5-ethoxy-2-methy1-2,3-
dihydrobenzofuran-6-yl)-2,2,2-trifluoroethoxy)pyrimidin-4-
yl)phenyl)propanoic acid
(2S)-2-amino-3-(4-(2-amino-6-(1-(benzofuran-5-yl)-2,2,2-
trifluoroethoxy)pyrimidin-4-yl)phenyl)propanoic acid
(2S)-2-amino-3-(4-(2-amino-6-(2,2,2-trifluoro-1-(2-m-
tolylfuran-3-yl)ethoxy)pyrimidin-4-yl)phenyl)propanoic acid
(S)-ethyl 3-(4-(2-amino-6-((R)-2,2,2-trifluoro-1-(3′-
methoxybiphenyl-4-yl)ethoxy)pyrimidin-4-yl)phenyl)-2-(2-
aminoacetamido)propanoate
(2S)-2-amino-3-(4-(6-(2,2,2-trifluoro-1-(2-(4-methylthiophen-3-
yl)phenyl)ethoxy)pyrazin-2-yl)phenyl)propanoic acid
(2S)-2-amino-3-(4-(2-amino-6-(2,2,2-trifluoro-1-(5-methyl-3-
phenylisoxazol-4-yl)ethoxy)pyrimidin-4-yl)phenyl)propanoic
acid
(S)-2-amino-3-(4-(2-amino-6-(3-(methylthio)phenyl)pyrimidin-
4-yl)phenyl)propanoic acid
(2S)-2-amino-3-(4-(2-amino-6-(2,2,2-trifluoro-1-(3′-
(methylthio)biphenyl-2-yl)ethoxy)pyrimidin-4-
yl)phenyl)propanoic acid
(2S)-2-amino-3-(4-(2-amino-6-(1-(3′-
((dimethylamino)methyl)biphenyl-2-yl)-2,2,2-
trifluoroethoxy)pyrimidin-4-yl)phenyl)propanoic acid
(S)-2-amino-3-(4-(2-amino-6-(3-
(trifluoromethoxy)phenyl)pyrimidin-4-yl)phenyl)propanoic acid
(2S)-2-amino-3-(4-(2-amino-6-(2,2,2-trifluoro-1-(3′-
(trifluoromethoxy)biphenyl-2-yl)ethoxy)pyrimidin-4-
yl)phenyl)propanoic acid
(S)-3-(4-(2-amino-6-((R)-2,2,2-trifluoro-1-(3′-methoxybiphenyl-
4-yl)ethoxy)pyrimidin-4-yl)phenyl)-2-(2-
aminoacetamido)propanoic acid
(2S)-2-amino-3-(4-(2-amino-6-(2,2,2-trifluoro-1-(1-methyl-5-
phenyl-1H-pyrazol-4-yl)ethoxy)pyrimidin-4-
yl)phenyl)propanoic acid
(2S)-2-amino-3-(4-(2-amino-6-(2,2,2-trifluoro-1-(4-
(methylsulfonyl)phenyl)ethoxy)pyrimidin-4-
yl)phenyl)propanoic acid
(S)-2-amino-3-(4-(2-amino-6-((R)-1-(3′-
(dimethylamino)biphenyl-2-yl)-2,2,2-trifluoroethoxy)pyrimidin-
4-yl)phenyl)propanoic acid
(2S)-2-amino-3-(4-(2-amino-6-(1-(2-chloro-4-
(methylsulfonyl)phenyl)-2,2,2-trifluoroethoxy)pyrimidin-4-
yl)phenyl)propanoic acid
(2S)-2-amino-3-(4-(2-amino-6-(2,2,2-trifluoro-1-(3-(furan-2-
yl)thiophen-2-yl)ethoxy)pyrimidin-4-yl)phenyl)propanoic acid
(2S)-2-amino-3-(4-(2-amino-6-(1-(2-(cyclopentyloxy)-4-
fluorophenyl)-2,2,2-trifluoroethoxy)pyrimidin-4-
yl)phenyl)propanoic acid
(2S)-2-amino-3-(4-(2-amino-6-(2,2,2-trifluoro-1-(2-(3-
methoxyphenyl)cyclohex-1-enyl)ethoxy)pyrimidin-4-
yl)phenyl)propanoic acid
(2S)-2-amino-3-(4-(2-amino-6-(2,2,2-trifluoro-1-(pyrimidin-5-
yl)ethoxy)pyrimidin-4-yl)phenyl)propanoic acid
(2S)-2-amino-3-(4-(5-(2,2,2-trifluoro-1-(3′-methoxybiphenyl-3-
yl)ethoxy)pyrazin-2-yl)phenyl)propanoic acid
(S)-2-amino-3-(4-(2-amino-6-((S)-1-(3′-
(dimethylamino)biphenyl-2-yl)-2,2,2-trifluoroethoxy)pyrimidin-
4-yl)phenyl)propanoic acid
(2S)-2-amino-3-(4-(2-amino-6-(2,2,2-trifluoro-1-(2-(furan-2-
carboxamido)pheny)ethoxy)pyrimidin-4-yl)phenyl)propanoic
acid
(2S)-2-amino-3-(4-(2-amino-6-(1-(4-chloro-2-
(methylsulfonyl)phenyl)-2,2,2-trifluoroethoxy)pyrimidin-4-
yl)phenyl)propanoic acid
(S)-isopropyl 2-amino-3-(4-(2-amino-6-((R)-2,2,2-trifluoro-1-
(3′-methoxybiphenyl-4-yl)ethoxy)pyrimidin-4-
yl)phenyl)propanoate
(2S)-2-amino-3-(4-(6-(1-(2-(cyclopentyloxy)-4-fluorophenyl)-
2,2,2-trifluoroethoxy)pyrimidin-4-yl)phenyl)propanoic acid
(2S)-2-amino-3-(4-(6-(1-(2-(cyclohexyloxy)-4-fluoropheny1)-
2,2,2-trifluoroethoxy)pyrimidin-4-yl)phenyl)propanoic acid
(2S)-2-amino-3-(4-(2-amino-6-(2,2,2-trifluoro-1-(1-(thiophen-2-
yl)cyclohexyl)ethoxy)pyrimidin-4-yl)phenyl)propanoic acid
(2S)-2-amino-3-(4-(2-(2,2,2-trifluoro-1-(3'-methoxybipheny1-4-
yl)ethoxy)thiazol-5-yl)phenyl)propanoic acid
(2S)-2-amino-3-(4-(2-amino-6-(1-(2-(cyclohexyloxy)-4-
fluoropheny1)-2,2,2-trifluoroethoxy)pyrimidin-4-
yl)phenyl)propanoic acid
(2S)-2-amino-3-(4-(2-amino-6-(2,2,2-trifluoro-1-(1-(4-
methoxyphenyl)cyclohexyl)ethoxy)pyrimidin-4-
yl)phenyl)propanoic acid
(2S)-2-amino-3-(4-(6-(2,2,2-trifluoro-1-(4-fluoro-2-
methylphenyl)ethoxy)pyrimidin-4-yl)phenyl)propanoic acid
(2S)-2-amino-3-(4-(2-amino-6-(2,2,2-trifluoro-1-(4-fluoro-2-
methylphenyl)ethoxy)pyrimidin-4-yl)phenyl)propanoic acid
(2S)-2-amino-3-(4-(2-amino-6-(oxazol-2-
yl(phenyl)methoxy)pyrimidin-4-yl)phenyl)propanoic acid
(S)-2-amino-3-(4-(2-amino-6-(1-cyclohexyl-2,2,2-
trifluoroethylideneaminooxy)pyrimidin-4-yl)phenyl)propanoic
acid
(2S)-2-amino-3-(4-(2-amino-6-(1-(2-(3-
(dimethylainino)phenyl)furan-3-y1)-2,2,2-
trifluoroethoxy)pyrimidin-4-yl)phenyl)propanoic acid
(2S)-2-amino-3-(4-(2-amino-6-(2,2,2-trifluoro-1-(5-
phenylthiophen-2-yl)ethoxy)pyrimidin-4-yl)phenyl)propanoic
acid
(S)-phenyl 2-amino-3-(4-(2-amino-6-((R)-2,2,2-trifluoro-1-(3′-
methoxybiphenyl-4-yl)ethoxy)pyrimidin-4-
yl)phenyl)propanoate
(S)-2-amino-3-(4-(2-amino-6-((R)-1-(3′-
((dimethylamino)methyl)biphenyl-4-yl)-2,2,2-
trifluoroethoxy)pyrimidin-4-yl)phenyl)propanoic acid
(S)-2-amino-3-(4-(1-(3-methoxybenzoyl)-1H-pyrazol-4-
yl)phenyl)propanoic acid
(2S)-2-amino-3-(4-(6-(2,2,2-trifluoro-1-(5-phenylfuran-2-
yl)ethoxy)pyrimidin-4-yl)phenyl)propanoic acid
(2S)-2-amino-3-(4-(2-amino-6-(1-(4-chloro-2-fluorophenyl)-
2,2,2-trifluoroethoxy)pyrimidin-4-yl)phenyl)propanoic acid
(S,E)-2-amino-3-(4-(2-amino-6-(4-
(trifluoromethyl)styryl)pyrimidin-4-yl)phenyl)propanoic acid
(2S)-2-amino-3-(4-(2-amino-6-(1-(3,4-dichlorophenyl)-2,2,2-
trifluoroethoxy)pyrimidin-4-yl)phenyl)propanoic acid
(2S)-2-amino-3-(4-(2-amino-6-(1-(4-chloro-3-fluoropheny1)-
2,2,2-trifluoroethoxy)pyrimidin-4-yl)phenyl)propanoic acid
(S)-2-amino-3-(4-(2-amino-6-((R)-1-(3′-
(dimethylamino)biphenyl-4-yl)-2,2,2-trifluoroethoxy)pyrimidin-
4-y1)phenyl)propanoic acid
(2S)-2-amino-3-(4-(2-amino-6-(1-chloro-2,2,2-trifluoro-1-(4-
methoxybiphenyl-2-yl)ethoxy)pyrimidin-4-y1)phenyl)propanoic
acid
(2S)-2-amino-3-(4-(6-(2,2,2-trifluoro-1-(5-phenylthiophen-2-
yl)ethoxy)pyrimidin-4-yl)phenyl)propanoic acid
(S)-2-amino-3-(4-(5-(4-phenoxyphenyl)-1H-1,2,3-triazol-1-
yl)phenyl)propanoic acid
(S,E)-2-amino-3-(4-(2-amino-6-(2-(bipheny1-4-
yl)vinyl)pyrimidin-4-yl)phenyl)propanoic acid
(S)-2-amino-3-(4-(4-amino-6-((R)-2,2,2-trifluoro-1-(3′-
methoxybiphenyl-4-yl)ethoxy)pyrimidin-2-yl)phenyl)propanoic
acid
(S)-2-amino-3-(4-(4′-methoxybiphenyl-4-
ylsulfonamido)phenyl)propanoic acid
(2S)-2-amino-3-(4-(2-amino-6-(2,2,2-trifluoro-1-(6-(3-
methoxypheny)pyridin-3-yl)ethoxy)pyrimidin-4-
yl)phenyl)propanoic acid
(2S)-2-amino-3-(4-(2-amino-6-(2,2,2-trifluoro-1-(6-(2-fluoro-3-
methoxyphenyl)pyridin-3-yl)ethoxy)pyrimidin-4-
yl)phenyl)propanoic acid
2-amino-3-(5-(4′-methylbiphenyl-4-yl)-1H-indol-3-yl)propanoic
acid
2-amino-3-(5-m-tolyl-1H-indol-3-yl)propanoic acid
(2S)-2-amino-3-(4-(2-(2-methoxyphenyl)furan-3-
carboxamido)phenyl)propanoic acid
2-amino-3-(5-(1-benzyl-1H-pyrazol-4-yl)-1H-indol-3-
yl)propanoic acid
(2S)-2-amino-3-(4-(2-amino-6-(2,2,2-trifluoro-1-(6-(thiophen-2-
yl)pyridin-3-yl)ethoxy)pyrimidin-4-yl)phenyl)propanoic acid
2-amino-3-(6-(1-benzyl-1H-pyrazol-4-yl)-1H-indol-3-
yl)propanoic acid
(S)-2-amino-3-(4-((2-(4-(trifluoromethyl)phenyl)thiazol-4-
yl)methylamino)phenyl)propanoic acid
(S)-2-amino-3-(4-((4′-methoxybiphenyl-4
ylsulfonamido)methyl)phenyl)propanoic acid
(S)-2-amino-3-(4-(3-(2-methoxydibenzo[b,d]furan-3-
yl)ureido)phenyl)propanoic acid
(S)-2-amino-3-(4-(3-(2,2-
diphenylethyl)ureido)pheny)propanoic acid
(S)-2-amino-3-(4-(phenylethynyl)phenyl)propanoic acid
(S)-2-amino-3-(4-(2-amino-6-((5-(1-methyl-5-(trifluoromethyl)-
1H-pyrazol-3-yl)thiophen-2-yl)methoxy)pyrimidin-4-
yl)phenyl)propanoic acid
(2S)-2-amino-3-(4-(2-amino-6-(1,1,1-trifluoro-3-((R)-2,2,3-
trimethylcyclopent-3-enyl)propan-2-yloxy)pyrimidin-4-
yl)phenyl)propanoic acid
(2S)-2-amino-3-(4-(2-amino-6-(3-(2-
hydroxyethylcarbamoyl)piperidin-1-yl)pyrimidin-4-
yl)phenyl)propanoic acid
(2S)-2-amino-3-(4-(2-amino-6-(3-(pyridin-2-yloxy)piperidin-1-
yl)pyrimidin-4-yl)phenyl)propanoic acid
(S)-2-amino-3-(4-(2-amino-6-(4-chloro-3-(piperidine-1-
carbonyl)phenyl)pyrimidin-4-yl)phenyl)propanoic acid

In certain embodiments, the TPH1 inhibitors disclosed herein reduce serum or plasma serotonin to a level that is at least about 10% less than the level before treatment with the TPH1 inhibitor. In certain embodiments, the TPH1 inhibitor reduces serum or plasma serotonin to a level that is about 10% less, about 20% less, about 30% less, about 40% less, about 50% less, about 60% less, about 70% less, about 80% less, or about 90% less, than the level before treatment with the TPH1 inhibitor.

Synthesis of the compounds described herein can be carried out by methods similar to those disclosed in U.S. Patent Application Publication US 2007/0191370, U.S. Patent Application Publication US 2008/0153852, U.S. Patent Application Publication US 2009/0005381, and U.S. Patent Application Publication US 2009/0029993. Moieties such as A, X, D, and E can be prepared and linked according to the methods described in those patent applications. By choosing suitable starting materials for the remaining portion of the structures disclosed herein, the remaining portion can be incorporated with the A-X-D or A-X-D-E portion in the final structure and thus the compounds disclosed herein can be prepared.

One skilled in the art would be guided by other publications. For instance, one skilled in the art could consult the Examples in International Patent Publication WO 2010/056992 and could, for example, choose intermediates such as the following from Scheme 1 of Example 9

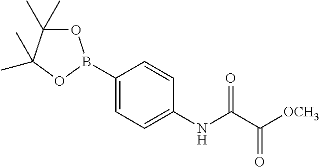 [SMILES: COC(=O)C(=O)Nc1ccc(B2OC(C)(C)C(C)(C)O2)cc1]

and link the intermediate compounds to suitable moieties such as A, X₁, D, and E that had been prepared according to the disclosures of the patent applications described above. By choosing other intermediates similar to the intermediate shown above, one skilled in the art could readily synthesize other TPH1 inhibitors disclosed herein.

Synthesis of specific compounds disclosed herein as well as the compounds within the generic formulas disclosed herein can also be carried out by methods similar to those disclosed in International Patent Publication WO 2007/089335 and International Patent Publication WO 2008/073933. Moieties such as A, X, D, and E can be prepared and linked according to the methods described in WO 2007/089335, in particular the methods disclosed at pages 35-41. Further methods that can be turned to for guidance are shown on pages 14-17 of WO 2008/073933. By choosing suitable starting materials for the remaining portion of the structures disclosed herein (e.g., the X¹-2-oxoacetate or X¹-2-oxoacetic acid moiety in certain of the generic formulas described above), the remaining portion can be incorporated with the A-X-D or A-X-D-E portion in the final structure and thus the compounds of the present invention can be prepared.

Certain compounds disclosed herein can be prepared according to the methods disclosed in International Patent Publication WO 2009/123978 or International Patent Publication WO 2010/056992, incorporated herein by reference in their entireties and specifically for the purpose of their disclosures of the synthesis of the compounds disclosed herein.

The present invention also encompasses the use of certain derivatives of the TPH1 inhibitors disclosed herein. For example, prodrugs of the TPH1 inhibitors could be produced by esterifying the carboxylic acid functions of the TPH1 inhibitors with a lower alcohol, e.g., methanol, ethanol, propanol, isopropanol, butanol, etc. The use of prodrugs of the TPH1 inhibitors that are not esters is also contemplated. For example, pharmaceutically acceptable carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters of the TPH1 inhibitors are also contemplated. In some embodiments, the prodrugs will contain a biohydrolyzable moiety (e.g., a biohydrolyzable amide, biohydrolyzable carbamate, biohydrolyzable carbonate, biohydrolyzable ester, biohydrolyzable phosphate, or biohydrolyzable ureide analog). Guidance for the preparation of prodrugs of the TPH1 inhibitors disclosed herein can be found in publications such as Design of Prodrugs, Bundgaard, A. Ed., Elsevier, 1985; Design and Application of Prodrugs, A Textbook of Drug Design and Development, Krosgaard-Larsen and H. Bundgaard, Ed., 1991, Chapter 5, pages 113-191; and Bundgaard, H., Advanced Drug Delivery Review, 1992, 8, pages 1-38.

In certain embodiments, the TPH1 inhibitor inhibits TPH1 without significantly affecting the level of brain-derived serotonin. Methods of obtaining such inhibitors include: (1) screening for compounds that inhibit TPH1 to a much greater extent than TPH2; and (2) screening for compounds that, while they inhibit both TPH1 and TPH2, cannot cross the blood brain barrier and thus are effectively specific for TPH1 when administered to the patient outside the central nervous system. Of course, compounds that both inhibit TPH1 to a much greater extent than TPH2 and cannot cross the blood brain barrier are also suitable. Preferably, compounds that inhibit TPH1 to a much greater extent than TPH2 have an IC₅₀ for TPH2 that is at least about 10-fold greater than their IC₅₀ for TPH1.

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

A chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol, and pyridin-4-ol.

If the stereochemistry of a structure or a portion of a structure is not indicated with, for example, bold or dashed lines or wedges, the structure or the portion of the structure is to be interpreted as encompassing all stereoisomers of it, unless the chemical name associated with the structure indicates otherwise. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences. When the stereochemistry of a structure or a portion of a structure is indicated with, for example, a bold or dashed line, the use of that structure in the methods describe herein encompasses the use of the indicated stereochemistry substantially free of any of the non-indicated structure. For example, such use includes the use of the indicated structure where the indicated structure is present in an enantiomeric excess of 95%, 96%, 97%, 98%, 99%, or 99.5%,

Notwithstanding the above, it is understood that the methods of the invention may encompass the use of pure R and S enantiomers of the compounds disclosed herein as well as racemic mixtures. Thus, the disclose of the use of a compound without indication of any particular stereochemistry should be considered a disclosure of the use of that compound in the form of all racemic mixtures (e.g., a mixture of about 50% R and 50% S enantiomers) as well as a disclosure of the use of essentially pure enantiomers (i.e., about 100% R or about 100% S enantiomers).

In certain embodiments of the invention, a therapeutically effective amount of one or more of the TPH1 inhibitors described herein is administered in combination with other compounds that are known to prevent or treat hyperlipidemia or atherosclerosis to a subject who has or is at risk of developing hyperlipidemia or atherosclerosis in order to treat or prevent hyperlipidemia or atherosclerosis.

In certain embodiments, the TPH1 inhibitor is administered in combination with an inhibitor of hormone sensitive lipase (HSL). For example, the TPH1 inhibitor may be administered with one or more of the following HSL inhibitors:

Methylphenylcarbamic acid 5-(4-isobutyl-2,6,-dioxopiperazin-1-yl)pyridinyl-2-yl ester

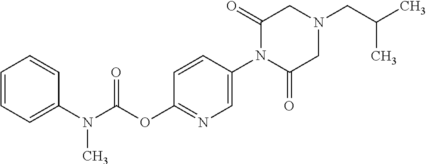 [SMILES: CC(C)CN1CC(=O)N(c2ccc(OC(=O)N(C)c3ccccc3)nc2)C(=O)C1]

### See International Patent Publication WO 2006/087308.

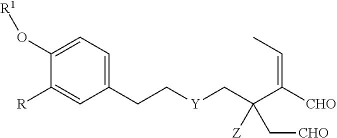

wherein R is hydrogen, hydroxyl or —OR¹;

wherein R¹ is hydrogen or a chemical moiety that can be cleaved in vivo to release a hydroxyl group and includes, for example, aliphatic or aromatic acyl (to form an ester bond) and the like. Such aliphatic or aromatic groups can include a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group, a carbalkoxy, a carbaryloxy, —SO₃H and —SO₃R³, —P(O)(OH)₂, or —P(O)(OR³)₂;

wherein R³ is a saturated or unsaturated aliphatic group, substituted or unsubstituted aliphatic group, substituted or unsubstituted saturated or unsaturated alicyclic group, substituted or unsubstituted aromatic group, substituted or unsubstituted heteroaromatic group, substituted or unsubstituted heterocyclic group, glucuronide or glucuronide ester, —P(O)(OH)₂, and —P(O)(OR³)₂;

wherein Y is —(CH₂)ₘ— where m=1 or 2, —OC(O)—, —O(CH₂)ₘ— where m=1 or 2, or —S(O)ₙ(CH₂)ₘ— where m=1 or 2; n=0, 1 or 2;

wherein Z is hydrogen, CH₃, F, Cl, Br or I.

### See International Patent Publication WO 2007/081808.

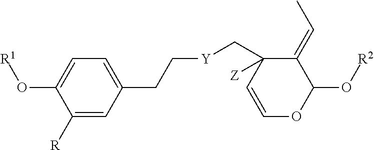

wherein R, R¹, R³, Y and Z are as defined in the previous paragraph; and,

wherein R² is hydrogen or a hydroxyl protecting group, as described in “Protective Groups in Organic Synthesis” by Therodora W. Greene, Peter G. M. Wuts, 1999, 3^{rd} edition, pp 17-200, —SO₃H and —SO₃R³ where R³ is a saturated or unsaturated aliphatic group; a substituted or unsubstituted saturated or unsaturated alicyclic group.

### See International Patent Publication WO 2007/081808.

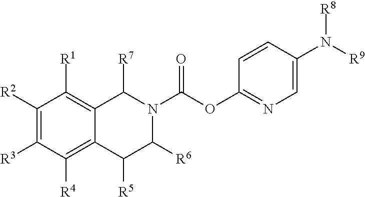

wherein R¹, R², R³, R⁴, R⁶, R⁶, and R⁷, independent of each other, each represents hydrogen, hydroxy, mercapto, amino, —CONH₂, —CSNH₂, —NH—CO—NH₂, —NH—CS—NH₂, halogen, —S(═O)₂(OH), C₁₋₆-alkyl, C₁₋₆-alkoxy, C₂₋₆-alkenyl, aryl, heteroaryl, C₃₋₈-heterocyclyl and C₃₋₁₃-cycloalkyl, wherein each of hydroxy, mercapto, amino, —CONH₂, —NH—CO—NH₂, —NH—CS—NH₂, —CSNH₂, —S(═O)₂(OH), C₁₋₆-alkyl, C₁₋₆-alkoxy, C₂₋₆-alkenyl, aryl, heteroaryl, C₃₋₈-heterocyclyl and C₃₋₁₃-cycloalkyl may optionally be substituted with one or more substituents independently selected from hydroxy, mercapto, oxo (═O), thioxo (═S), halogen, amino, —S(═O)₂(OH), C₁₋₆-alkyl, C₁₋₆-alkoxy, C₂₋₆-alkenyl, aryl, heteroaryl, C₃₋₈-heterocycyl, and C₃₋₁₃-cycloalkyl, wherein each of hydroxy, mercapto, —S(═O)₂(OH), C₁₋₆-alkyl, C₁₋₆-alkoxy, C₂₋₆-alkenyl, aryl, heteroaryl, C₃₋₈-heterocyclyl and C₃₋₁₃-cycloalkyl may optionally be substituted with one or more substituents independently selected from hydroxy, mercapto, oxo, halogen, amino, —S(═O)₂(OH), halo-C₁₋₄-alkyl, halo-C₁₋₄-alkoxy, C₁₋₆-alkyl, C₁₋₆-alkoxy, C₂₋₆-alkenyl, aryl, heteroaryl, C₃₋₈-heterocyclyl, and C₃₋₁₃-cycloalkyl; and either R⁸ is hydrogen and R⁹ represents C₃₋₈-heterocyclyl which, optionally, is substituted with one or more substituents independently selected from hydroxy, mercapto, oxo (═O), thioxo (═S), halogen, amino, —S(═O)₂(OH), C₁₋₆-alkyl, C₁₋₆-alkoxy, C₂₋₆-alkenyl, aryl, heteroaryl, C₃₋₈-heterocyclyl, and C₃₋₁₃-cycloalkyl; or R⁸ together with R⁹ and together with the adjacent nitrogen atom represents C₃₋₈-heterocyclyl which, optionally, is substituted with one or more substituents independently selected from hydroxy, mercapto, oxo (═O), thioxo (═S), halogen, amino, —S(═O)₂(OH), C₁₋₆-alkyl, C₁₋₆-alkoxy, C₂₋₆-alkenyl, aryl, heteroaryl, C₃₋₈-heterocycyl, and C₃₋₁₃-cycloalkyl; or a pharmaceutically acceptable salt thereof, or a pharmaceutically acceptable solvate thereof, or any tautomeric forms, stereoisomers, mixture of stereoisomers including a racemic mixture, or polymorphs.

### See International Patent Publication WO 2006/087309.

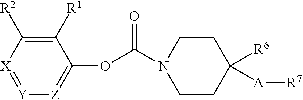

wherein
R¹ and R² are independently selected from hydrogen, hydroxy, sulfanyl, amino, halogen, sulfo, Cl₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₄-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

### X is N or C—R³; Y is N or C—R⁴; Z Is N or C—R⁶;

R³, R⁴ and R⁵ are independently selected from hydrogen, hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocycyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R⁸ is hydrogen or fluor;

A is selected from —O—, —S—, —S(═O)—, —S(═O)₂—, —CH₂O—, —CH₂S—, —CH₂CH₂—N(R⁸)—, —CH₂CHF—N(R⁸)—, —CH₂CF₂—N(R⁸)—, and —CHFCH₂—N(R⁸)—;

R⁸ is selected from hydrogen, C₁₋₆-alkyl, C₂₋₆-alkenyl, aryl, heteroaryl, C₃₋₈-heterocyclyl and C₃₋₁₀-cycloalkyl, wherein each of C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocycyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocycyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
R⁷ is selected from aryl or heteroaryl, which may optionally be substituted with one or more substituents selected from hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocycyl and C₃₋₁₀-cycloalkyl is optionally substituted with one or more substituents independently selected from hydroxy, sulfanyl, oxo, halogen, amino, C₁₋₆ alkyl, perhalomethyl and perhalomethoxy;

### See International Patent Publication WO 2004/111032.

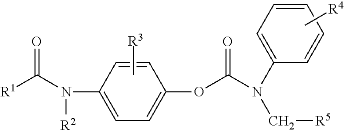

wherein
R¹ and R² are independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R³, R⁴ and R⁵ are independently selected from hydrogen, hydroxy, sulfanyl, fluor, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;

### See International Patent Publication WO 2004/111025.

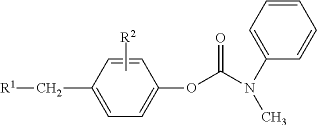

wherein
R¹ is selected from hydroxy, sulfanyl, sulfa, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₂₋₆-alkenyl, aryl, heteroaryl, C₃₋₈-heterocyclyl, and C₃₋₁₀-cycloalkyl;
R² is selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfa, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy; C₂₋₆-alkenyl, aryl, heteroaryl, C₃₋₈-heterocyclyl, and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl may optionally, be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

### See International Patent Publication WO 2004/111006.

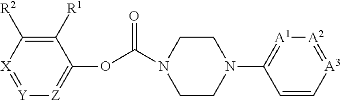

wherein

### X is N or C—R³, Y is N or C—R⁴, Z is N or C—R⁵;

A¹ is N or C—R⁶, A² is N or C—R⁷, A³ is N or C—R⁶; provided that at least one of A¹, A² and A³ is N;
R¹, R², R³, R⁴, R⁵, R⁶, R⁷ and R⁸ are independently selected from hydrogen, hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₆-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocycyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₂₋₆-alkenyl, perhalomethyl and perhalomethoxy;

### See International Patent Publication WO 2004/111007.

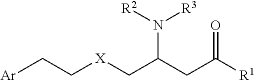

wherein:

Ar is an aryl or heteroaryl group;

X is —OC(O)—, —NR⁶C(O)—, —(CH₂)ₘ—, —O(CH₂)ₘ, —S(O)(CH₂)ₘ, or —S(O)O(CH₂)ₘ, wherein m is 1 or 2;

R¹ is selected from the group consisting of hydrogen. OH, C₁₋₁₀alkyl, aryl, heteroaryl, OC₁₋₁₀alkyl. O-aryl, O-heteroaryl, OC₁₋₁₀alkylenylaryl, OC₁₋₁₀alkylenylheteroaryl, and N(R⁴)R⁵;

R², R³, R⁴, R⁵, and R⁶ are each independently selected from the group consisting of hydrogen, C₁₋₁₀alkyl, C(O)C₁₋₁₀alkyl, C(O)C(O)C₁₋₁₀alkyl, C(O)NR⁷R⁸, and C(O)C₁₋₁₀haloalkyl; and

R⁷ and R⁸ are independently selected from the group consisting of hydrogen, C₁₋₁₀alkyl, aryl, and heteroaryl,

or a salt thereof.

In certain embodiments, the TPH1 inhibitor may be administered with one or more of the HSL inhibitors disclosed in International Patent Publication WO 2006/074957; International Patent Publication WO 2005/073199; International Patent Publication WO 2004/111031; International Patent Publication WO 2004/111004; International Patent Publication WO 2004/035550; International Patent Publication WO 2003/051841; International Patent Publication WO 2003/051842; or International Patent Publication WO 2001/066531.

The efficacy of therapy to prevent or treat hyperlipidemia or atherosclerosis by administering TPH1 inhibitors can be monitored by measuring plasma lipid levels (e.g., levels of cholesterol, triglycerides, glycerol, or free fatty acids) or by monitoring arterial plaque (e.g., by angiogram) before and over time after treatment to determine the efficacy of the therapy.

The amount of therapeutic agents such as TPH1 inhibitors disclosed herein to be administered to a patient depends on many factors, as discussed herein. However, in humans, for example, the amount may range from about 1 mg/day to about 2 g/day; preferably from about 15 mg/day to about 500 mg/day; or from about 20 mg/day to about 250 mg/day; or from about 40 mg/day to about 100 mg/day. Other preferred dosages include about 2 mg/day, about 5 mg/day, about 10 mg/day, about 15 mg/day, about 20 mg/day, about 25 mg/day, about 30 mg/day, about 40 mg/day, about 50 mg/day, about 60 mg/day, about 70 mg/day, about 80 mg/day, about 90 mg/day, about 100 mg/day, about 125 mg/day, about 150 mg/day, about 175 mg/day, about 200 mg/day, about 250 mg/day, about 300 mg/day, about 350 mg/day, about 400 mg/day, about 500 mg/day, about 600 mg/day, about 700 mg/day, about 800 mg/day, and about 900 mg/day.

Other dose ranges that may be used include from about 50 mg/day to about 15 g/day; from about 50 mg/day to about 10 g/day; from about 50 mg/day to about 5 g/day; from about 50 mg/day to about 1 g/day; from about 50 mg/day to about 900 mg/day; from about 50 mg/day to about 800 mg/day; from about 50 mg/day to about 700 mg/day; from about 50 mg/day to about 600 mg/day; from about 50 mg/day to about 500 mg/day; from about 50 mg/day to about 400 mg/day; from about 50 mg/day to about 300 mg/day; or from about 50 mg/day to about 200 mg/day.

Other dose ranges that may be used include from about 100 mg/day to about 15 g/day; from about 100 mg/day to about 10 g/day; from about 100 mg/day to about 5 g/day; from about 100 mg/day to about 1 g/day; from about 100 mg/day to about 900 mg/day; from about 100 mg/day to about 800 mg/day; from about 100 mg/day to about 700 mg/day; from about 100 mg/day to about 600 mg/day; from about 100 mg/day to about 500 mg/day; from about 100 mg/day to about 400 mg/day; from about 100 mg/day to about 300 mg/day; or from about 100 mg/day to about 200 mg/day.

Other dose ranges that may be used include from about 200 mg/day to about 15 g/day; from about 200 mg/day to about 10 g/day; from about 200 mg/day to about 5 g/day; from about 200 mg/day to about 1 g/day; from about 200 mg/day to about 900 mg/day; from about 200 mg/day to about 800 mg/day; from about 200 mg/day to about 700 mg/day; from about 200 mg/day to about 600 mg/day; from about 200 mg/day to about 500 mg/day; from about 200 mg/day to about 400 mg/day; or from about 200 mg/day to about 300 mg/day.

Other dose ranges that may be used include from about 300 mg/day to about 15 g/day; from about 300 mg/day to about 10 g/day; from about 300 mg/day to about 5 g/day; from about 300 mg/day to about 1 g/day; from about 300 mg/day to about 900 mg/day; from about 300 mg/day to about 800 mg/day; from about 300 mg/day to about 700 mg/day; from about 300 mg/day to about 600 mg/day; from about 300 mg/day to about 500 mg/day; or from about 300 mg/day to about 400 mg/day.

Other dose ranges that may be used include from about 400 mg/day to about 15 g/day; from about 400 mg/day to about 10 g/day; from about 400 mg/day to about 5 g/day; from about 400 mg/day to about 1 g/day; from about 400 mg/day to about 900 mg/day; from about 400 mg/day to about 800 mg/day; from about 400 mg/day to about 700 mg/day; from about 400 mg/day to about 600 mg/day; or from about 400 mg/day to about 500 mg/day.

Other dose ranges that may be used include from about 500 mg/day to about 15 g/day; from about 500 mg/day to about 10 g/day; from about 500 mg/day to about 5 g/day; from about 500 mg/day to about 4 g/day; from about 500 mg/day to about 3 g/day; from about 500 mg/day to about 2 g/day; from about 500 mg/day to about 1 g/day; from about 500 mg/day to about 900 mg/day; from about 500 mg/day to about 800 mg/day; from about 500 mg/day to about 700 mg/day; or from about 500 mg/day to about 600 mg/day.

Other dose ranges that may be used include from about 600 mg/day to about 15 g/day; from about 600 mg/day to about 10 g/day; from about 600 mg/day to about 5 g/day; from about 600 mg/day to about 4 g/day; from about 600 mg/day to about 3 g/day; from about 600 mg/day to about 2 g/day; from about 600 mg/day to about 1 g/day; from about 600 mg/day to about 900 mg/day; from about 600 mg/day to about 800 mg/day; or from about 600 mg/day to about 700 mg/day.

Other dose ranges that may be used include from about 700 mg/day to about 15 g/day; from about 700 mg/day to about 10 g/day; from about 700 mg/day to about 5 g/day; from about 700 mg/day to about 4 g/day; from about 700 mg/day to about 3 g/day; from about 700 mg/day to about 2 g/day; from about 700 mg/day to about 1 g/day; from about 700 mg/day to about 900 mg/day; or from about 700 mg/day to about 800 mg/day.

Other dose ranges that may be used include from about 800 mg/day to about 15 g/day; from about 800 mg/day to about 10 g/day; from about 800 mg/day to about 5 g/day; from about 800 mg/day to about 4 g/day; from about 800 mg/day to about 3 g/day; from about 800 mg/day to about 2 g/day; from about 800 mg/day to about 1 g/day; or from about 800 mg/day to about 900 mg/day.

Other dose ranges that may be used include from about 900 mg/day to about 15 g/day; from about 900 mg/day to about 10 g/day; from about 900 mg/day to about 5 g/day; from about 900 mg/day to about 4 g/day; from about 900 mg/day to about 3 g/day; from about 900 mg/day to about 2 g/day; or from about 900 mg/day to about 1 g/day.

Other dose ranges that may be used include from about 1 g/day to about 15 g/day; from about 1 g/day to about 10 g/day; from about 1 g/day to about 5 g/day; from about 1 g/day to about 4 g/day; from about 1 g/day to about 3 g/day; or from about 1 g/day to about 2 g/day.

Other dosages that may be used include from about 1 g/day, about 2 g/day, about 3 g/day, about 4 g/day, about 5 g/day, about 6 g/day, about 7 g/day, about 8 g/day, about 9 g/day, about 10 g/day, about 11 g/day, about 12 g/day, about 13 g/day, about 14 g/day, or about 15 g/day.

The amount of therapeutic agent disclosed herein to be administered to a patient may range from about 5 mg/kg/day to about 500 mg/kg/day, from about 5 mg/kg/day to about 400 mg/kg/day, from about 5 mg/kg/day to about 300 mg/kg/day, from about 5 mg/kg/day to about 250 mg/kg/day, from about 5 mg/kg/day to about 200 mg/kg/day, from about 5 mg/kg/day to about 150 mg/kg/day, from about 5 mg/kg/day to about 100 mg/kg/day, from about 5 mg/kg/day to about 75 mg/kg/day, from about 5 mg/kg/day to about 50 mg/kg/day, from about 5 mg/kg/day to about 40 mg/kg/day, from about 5 mg/kg/day to about 35 mg/kg/day, from about 5 mg/kg/day to about 30 mg/kg/day, from about 5 mg/kg/day to about 25 mg/kg/day, from about 5 mg/kg/day to about 24 mg/kg/day, from about 5 mg/kg/day to about 23 mg/kg/day, from about 5 mg/kg/day to about 22 mg/kg/day, from about 5 mg/kg/day to about 21 mg/kg/day, from about 5 mg/kg/day to about 20 mg/kg/day, from about 5 mg/kg/day to about 19 mg/kg/day, from about 5 mg/kg/day to about 18 mg/kg/day, from about 5 mg/kg/day to about 17 mg/kg/day, from about 5 mg/kg/day to about 16 mg/kg/day, from about 5 mg/kg/day to about 15 mg/kg/day, from about 5 mg/kg/day to about 14 mg/kg/day, from about 5 mg/kg/day to about 13 mg/kg/day, from about 5 mg/kg/day to about 12 mg/kg/day, from about 5 mg/kg/day to about 11 mg/kg/day, or from about 5 mg/kg/day to about 10 mg/kg/day.

Other dose ranges that may be used include from about 10 mg/kg/day to about 500 mg/kg/day, from about 10 mg/kg/day to about 400 mg/kg/day, from about 10 mg/kg/day to about 300 mg/kg/day, from about 10 mg/kg/day to about 250 mg/kg/day, from about 10 mg/kg/day to about 200 mg/kg/day, from about 10 mg/kg/day to about 150 mg/kg/day, from about 10 mg/kg/day to about 100 mg/kg/day, from about 10 mg/kg/day to about 75 mg/kg/day, from about 10 mg/kg/day to about 50 mg/kg/day, from about 10 mg/kg/day to about 45 mg/kg/day, from about 10 mg/kg/day to about 40 mg/kg/day, from about 10 mg/kg/day to about 35 mg/kg/day, from about 10 mg/kg/day to about 34 mg/kg/day, from about 10 mg/kg/day to about 33 mg/kg/day, from about 10 mg/kg/day to about 32 mg/kg/day, from about 10 mg/kg/day to about 31 mg/kg/day, from about 10 mg/kg/day to about 30 mg/kg/day, from about 10 mg/kg/day to about 29 mg/kg/day, from about 10 mg/kg/day to about 28 mg/kg/day, from about 10 mg/kg/day to about 27 mg/kg/day, from about 10 mg/kg/day to about 26 mg/kg/day, from about 10 mg/kg/day to about 25 mg/kg/day, from about 10 mg/kg/day to about 24 mg/kg/day, from about 10 mg/kg/day to about 23 mg/kg/day, from about 10 mg/kg/day to about 22 mg/kg/day, from about 10 mg/kg/day to about 21 mg/kg/day, from about 10 mg/kg/day to about 20 mg/kg/day, from about 10 mg/kg/day to about 19 mg/kg/day, from about 10 mg/kg/day to about 18 mg/kg/day, from about 10 mg/kg/day to about 17 mg/kg/day, from about 10 mg/kg/day to about 16 mg/kg/day, or from about 10 mg/kg/day to about 15 mg/kg/day.

Other dose ranges that may be used include from about 15 mg/kg/day to about 500 mg/kg/day, from about 15 mg/kg/day to about 400 mg/kg/day, from about 15 mg/kg/day to about 300 mg/kg/day, from about 15 mg/kg/day to about 250 mg/kg/day, from about 15 mg/kg/day to about 200 mg/kg/day, from about 15 mg/kg/day to about 150 mg/kg/day, from about 15 mg/kg/day to about 100 mg/kg/day, from about 15 mg/kg/day to about 75 mg/kg/day, from about 15 mg/kg/day to about 50 mg/kg/day, from about 15 mg/kg/day to about 40 mg/kg/day, from about 15 mg/kg/day to about 30 mg/kg/day, from about 15 mg/kg/day to about 25 mg/kg/day, or from about 15 mg/kg/day to about 20 mg/kg/day.

Other dose ranges that may be used include from about 20 mg/kg/day to about 500 mg/kg/day, from about 20 mg/kg/day to about 400 mg/kg/day, from about 20 mg/kg/day to about 300 mg/kg/day, from about 20 mg/kg/day to about 250 mg/kg/day, from about 20 mg/kg/day to about 200 mg/kg/day, from about 20 mg/kg/day to about 150 mg/kg/day, from about 20 mg/kg/day to about 100 mg/kg/day, from about 20 mg/kg/day to about 75 mg/kg/day, from about 20 mg/kg/day to about 50 mg/kg/day, from about 20 mg/kg/day to about 40 mg/kg/day, from about 20 mg/kg/day to about 30 mg/kg/day, or from about 20 mg/kg/day to about 25 mg/kg/day.

Other dose ranges that may be used include from about 25 mg/kg/day to about 500 mg/kg/day, from about 25 mg/kg/day to about 400 mg/kg/day, from about 25 mg/kg/day to about 300 mg/kg/day, from about 25 mg/kg/day to about 250 mg/kg/day, from about 25 mg/kg/day to about 200 mg/kg/day, from about 25 mg/kg/day to about 150 mg/kg/day, from about 25 mg/kg/day to about 100 mg/kg/day, from about 25 mg/kg/day to about 75 mg/kg/day, from about 25 mg/kg/day to about 50 mg/kg/day, from about 25 mg/kg/day to about 40 mg/kg/day, or from about 25 mg/kg/day to about 30 mg/kg/day.

Other dose ranges that may be used include from about 30 mg/kg/day to about 500 mg/kg/day, from about 30 mg/kg/day to about 400 mg/kg/day, from about 30 mg/kg/day to about 300 mg/kg/day, from about 30 mg/kg/day to about 250 mg/kg/day, from about 30 mg/kg/day to about 200 mg/kg/day, from about 30 mg/kg/day to about 150 mg/kg/day, from about 30 mg/kg/day to about 100 mg/kg/day, from about 30 mg/kg/day to about 75 mg/kg/day, from about 30 mg/kg/day to about 50 mg/kg/day, or from about 30 mg/kg/day to about 40 mg/kg/day.

Other dose ranges that may be used include from about 40 mg/kg/day to about 500 mg/kg/day, from about 40 mg/kg/day to about 400 mg/kg/day, from about 40 mg/kg/day to about 300 mg/kg/day, from about 40 mg/kg/day to about 250 mg/kg/day, from about 40 mg/kg/day to about 200 mg/kg/day, from about 40 mg/kg/day to about 150 mg/kg/day, from about 40 mg/kg/day to about 100 mg/kg/day, from about 40 mg/kg/day to about 75 mg/kg/day, from about 40 mg/kg/day to about 60 mg/kg/day, or from about 40 mg/kg/day to about 50 mg/kg/day.

Other dose ranges that may be used include from about 50 mg/kg/day to about 500 mg/kg/day, from about 50 mg/kg/day to about 400 mg/kg/day, from about 50 mg/kg/day to about 300 mg/kg/day, from about 50 mg/kg/day to about 250 mg/kg/day, from about 50 mg/kg/day to about 200 mg/kg/day, from about 50 mg/kg/day to about 175 mg/kg/day, from about 50 mg/kg/day to about 150 mg/kg/day, from about 50 mg/kg/day to about 125 mg/kg/day, from about 50 mg/kg/day to about 100 mg/kg/day, from about 50 mg/kg/day to about 75 mg/kg/day, or from about 50 mg/kg/day to about 60 mg/kg/day.

Other dose ranges that may be used include from about 60 mg/kg/day to about 500 mg/kg/day, from about 60 mg/kg/day to about 400 mg/kg/day, from about 60 mg/kg/day to about 300 mg/kg/day, from about 60 mg/kg/day to about 250 mg/kg/day, from about 60 mg/kg/day to about 200 mg/kg/day, from about 60 mg/kg/day to about 175 mg/kg/day, from about 60 mg/kg/day to about 150 mg/kg/day, from about 60 mg/kg/day to about 125 mg/kg/day, from about 60 mg/kg/day to about 100 mg/kg/day, or from about 60 mg/kg/day to about 75 mg/kg/day.

Other dose ranges that may be used include from about 70 mg/kg/day to about 500 mg/kg/day, from about 70 mg/kg/day to about 400 mg/kg/day, from about 70 mg/kg/day to about 300 mg/kg/day, from about 70 mg/kg/day to about 250 mg/kg/day, from about 70 mg/kg/day to about 200 mg/kg/day, from about 70 mg/kg/day to about 175 mg/kg/day, from about 70 mg/kg/day to about 150 mg/kg/day, from about 70 mg/kg/day to about 125 mg/kg/day, or from about 70 mg/kg/day to about 100 mg/kg/day.

Other dose ranges that may be used include from about 80 mg/kg/day to about 500 mg/kg/day, from about 80 mg/kg/day to about 400 mg/kg/day, from about 80 mg/kg/day to about 300 mg/kg/day, from about 80 mg/kg/day to about 250 mg/kg/day, from about 80 mg/kg/day to about 200 mg/kg/day, from about 80 mg/kg/day to about 175 mg/kg/day, from about 80 mg/kg/day to about 150 mg/kg/day, from about 80 mg/kg/day to about 125 mg/kg/day, or from about 80 mg/kg/day to about 100 mg/kg/day.

Other dose ranges that may be used include from about 90 mg/kg/day to about 500 mg/kg/day, from about 90 mg/kg/day to about 400 mg/kg/day, from about 90 mg/kg/day to about 300 mg/kg/day, from about 90 mg/kg/day to about 250 mg/kg/day, from about 90 mg/kg/day to about 200 mg/kg/day, from about 90 mg/kg/day to about 175 mg/kg/day, from about 90 mg/kg/day to about 150 mg/kg/day, from about 90 mg/kg/day to about 125 mg/kg/day, or from about 90 mg/kg/day to about 100 mg/kg/day.

Other dose ranges that may be used include from about 100 mg/kg/day to about 500 mg/kg/day, from about 100 mg/kg/day to about 400 mg/kg/day, from about 100 mg/kg/day to about 300 mg/kg/day, from about 100 mg/kg/day to about 250 mg/kg/day, from about 100 mg/kg/day to about 200 mg/kg/day, from about 100 mg/kg/day to about 175 mg/kg/day, from about 100 mg/kg/day to about 150 mg/kg/day, or from about 100 mg/kg/day to about 125 mg/kg/day.

Other dosages that may be used include about 5 mg/kg/day, about 6 mg/kg/day, about 7 mg/kg/day, about 8 mg/kg/day, about 9 mg/kg/day, about 10 mg/kg/day, about 11 mg/kg/day, about 12 mg/kg/day, about 13 mg/kg/day, about 14 mg/kg/day, about 15 mg/kg/day, about 16 mg/kg/day, about 17 mg/kg/day, about 18 mg/kg/day, about 19 mg/kg/day, about 20 mg/kg/day, about 21 mg/kg/day, about 22 mg/kg/day, about 23 mg/kg/day, about 24 mg/kg/day, about 25 mg/kg/day, about 26 mg/kg/day, about 27 mg/kg/day, about 28 mg/kg/day, about 29 mg/kg/day, about 30 mg/kg/day, about 31 mg/kg/day, about 32 mg/kg/day, about 33 mg/kg/day, about 34 mg/kg/day, about 35 mg/kg/day, about 36 mg/kg/day, about 37 mg/kg/day, about 38 mg/kg/day, about 39 mg/kg/day, about 40 mg/kg/day, about 45 mg/kg/day, about 50 mg/kg/day, about 60 mg/kg/day, about 70 mg/kg/day, about 80 mg/kg/day, about 90 mg/kg/day, about 100 mg/kg/day, about 125 mg/kg/day, about 150 mg/kg/day, about 175 mg/kg/day, about 200 mg/kg/day, about 250 mg/kg/day, or about 350 mg/kg/day.

The amounts and dosages of therapeutic agents disclosed above may be administered in the form of a once-per day pharmaceutical composition. Thus, e.g., the disclosure of 50 mg/day is to be taken as including the disclosure of a pharmaceutical composition comprising a therapeutic agent as disclosed herein where the pharmaceutical composition comprises 50 mg of the therapeutic agent.

Routine experimentation will determine the appropriate dosage for each patient and each therapeutic agent by monitoring the therapeutic agent's effect on serum or plasma serotonin levels, which can be frequently and easily monitored. The therapeutic agent can be administered once or multiple times per day. Serum or plasma serotonin levels can be monitored before and during therapy to determine the appropriate amount of therapeutic agent to administer to lower serum or plasma serotonin levels or bring serum or plasma serotonin levels to normal and to maintain normal levels over extended periods of time. In a particular embodiment, a patient is tested to determine if his/her serum or plasma serotonin levels are significantly elevated above normal levels (e.g., about 25% above) before administering treatment with one or more TPH1 inhibitors. The frequency of administration may vary from a single dose per day to multiple doses per day. Preferred routes of administration include oral, intravenous and intraperitoneal, but other forms of administration may be chosen as well.

In certain embodiments, the therapeutic agents of the invention act selectively on peripheral serotonin or are administered in an amount that decreases serum or plasma serotonin without increasing or decreasing brain-derived serotonin.

Monitoring the therapeutic efficacy of TPH1 inhibitors is straightforward, as one can administer the TPH1 inhibitors in an amount and for a duration that reduces serum or plasma serotonin levels, and over time decreases plasma levels of cholesterol, triglycerides, glycerol, and/or free fatty acids. Serum or plasma serotonin levels and plasma levels of cholesterol, triglycerides, glycerol, and/or free fatty acids can be easily measured. Monitoring serum or plasma serotonin is simple and can be done frequently during the course of therapy to establish the appropriate dose for each patient. Any method known in the art for assaying serum or plasma serotonin can be used. Measuring plasma levels of cholesterol, triglycerides, glycerol, and/or free fatty acids may also be done by methods known in the art.

In certain embodiments, the methods of the present invention comprise the step of identifying a patient in need of therapy for hyperlipidemia or atherosclerosis. Thus, the present invention provides a method comprising:

(a) identifying a patient in need of therapy for hyperlipidemia or atherosclerosis;

(b) administering to the patient a therapeutically effective amount of a therapeutic agent that decreases serum or plasma serotonin levels in order to prevent or treat hyperlipidemia or atherosclerosis in the patient identified in step (a).

In certain embodiments, “identifying” in step (a) above may be done by measuring the patient's level of serum or plasma serotonin, e.g., by forming a detectable complex of serum or plasma serotonin and a reagent that binds to serum or plasma serotonin in order to determine the patient's level of serum or plasma serotonin, where an elevated level of serum or plasma serotonin identifies the patient as being in need of therapy for hyperlipidemia or atherosclerosis. In certain embodiments, the reagent is an antibody or antibody fragment that binds to serotonin. In certain embodiments, the antibody or antibody fragment that binds to serotonin is labeled (e.g., radioactively, antigenically, fluorescently, with peroxidase etc.) and measuring the patient's level of serum or plasma serotonin includes the step of detecting a physical transformation in the label (e.g., radioactive decay of the label) or in a substance acted upon by the label (oxidation of a substrate by a peroxidase label).

In certain embodiments, “identifying” in step (a) above includes transforming serotonin from a bodily sample from the patient into a derivative of serotonin, e.g., N-acylserotonin. In certain embodiments, “identifying” in step (a) above includes subjecting serotonin or a serotonin derivative from a bodily sample from the patient to chromatography where the serotonin is separated from the components of the blood with which it is normally found and interacts with the stationary phase used in the chromatographic process.

In certain embodiments, the patient's level of serum or plasma serotonin is determined to be elevated in comparison to a standard level of serum or plasma serotonin that has previously been determined to be a normal level. In other embodiments, the patient's level of serum or plasma serotonin is determined to be elevated in comparison to a level of serum or plasma serotonin measured in a person who is known not to be in need of therapy for hyperlipidemia or atherosclerosis. In other embodiments, the patient's level of serum or plasma serotonin is determined to be elevated in comparison to a level of serum or plasma serotonin measured in the patient at a time when the patient was known not to be in need of therapy for hyperlipidemia or atherosclerosis.

In certain embodiments, “a patient in need of therapy for hyperlipidemia or atherosclerosis” is a patient with an elevated blood level of cholesterol. In such embodiments, “identifying” in step (a) above may be done by measuring the patient's level of blood cholesterol, e.g., by forming a detectable complex of cholesterol obtained from the blood of the patient and a reagent that binds to cholesterol in order to determine the patient's level of blood cholesterol, where an elevated level of blood cholesterol identifies the patient as being in need of therapy for hyperlipidemia or atherosclerosis. In certain embodiments, the reagent is an antibody or antibody fragment that binds to cholesterol. In certain embodiments, the antibody or antibody fragment that binds to cholesterol is labeled (e.g., radioactively, antigenically, fluorescently, with peroxidase etc.) and measuring the patient's level of blood cholesterol includes the step of detecting a physical transformation in the label (e.g., radioactive decay of the label) or in a substance acted upon by the label (oxidation of a substrate by a peroxidase label).

In certain embodiments, “identifying” in step (a) above includes transforming cholesterol from a bodily sample from the patient into a derivative of cholesterol, e.g., cholesterol esters or oxidation products of cholesterol. In certain embodiments, “identifying” in step (a) above includes subjecting cholesterol or a cholesterol derivative from a bodily sample from the patient to chromatography where the cholesterol is separated from the components of the blood with which it is normally found and interacts with the stationary phase used in the chromatographic process.

In certain embodiments, the patient's level of blood cholesterol is determined to be elevated in comparison to a standard level of blood cholesterol that has previously been determined to be a normal level. In other embodiments, the patient's level of blood cholesterol is determined to be elevated in comparison to a level of blood cholesterol measured in a person who is known not to be in need of therapy for hyperlipidemia or atherosclerosis. In other embodiments, the patient's level of blood cholesterol is determined to be elevated in comparison to a level of blood cholesterol measured in the patient at a time when the patient was known not to be in need of therapy for hyperlipidemia or atherosclerosis.

In certain embodiments, “a patient in need of therapy for hyperlipidemia or atherosclerosis” is a patient with an elevated blood level of triglycerides. In such embodiments, “identifying” in step (a) above may be done by measuring the patient's level of blood triglycerides, e.g., by forming a detectable complex of triglycerides obtained from the blood of the patient and a reagent that binds to triglycerides in order to determine the patient's level of blood triglycerides, where an elevated level of blood triglycerides identifies the patient as being in need of therapy for hyperlipidemia or atherosclerosis. In certain embodiments, the reagent is an antibody or antibody fragment that binds to triglycerides. In certain embodiments, the antibody or antibody fragment that binds to triglycerides is labeled (e.g., radioactively, antigenically, fluorescently, with peroxidase etc.) and measuring the patient's level of blood triglycerides includes the step of detecting a physical transformation in the label (e.g., radioactive decay of the label) or in a substance acted upon by the label (oxidation of a substrate by a peroxidase label).

In certain embodiments, “identifying” in step (a) above includes transforming triglycerides from a bodily sample from the patient into a derivative of triglycerides, e.g., transforming the triglycerides into glycerol and free fatty acids, as by enzymatic hydrolysis by lipase. In certain embodiments, “identifying” in step (a) above includes subjecting triglycerides or triglyceride derivatives from a bodily sample from the patient to chromatography where the triglycerides are separated from the components of the blood with which they are normally found and interact with the stationary phase used in the chromatographic process.

In certain embodiments, the patient's level of blood triglycerides is determined to be elevated in comparison to a standard level of blood triglycerides that has previously been determined to be a normal level. In other embodiments, the patient's level of blood triglycerides is determined to be elevated in comparison to a level of blood triglycerides measured in a person who is known not to be in need of therapy for hyperlipidemia or atherosclerosis. In other embodiments, the patient's level of blood triglycerides is determined to be elevated in comparison to a level of blood triglycerides measured in the patient at a time when the patient was known not to be in need of therapy for hyperlipidemia or atherosclerosis.

In certain embodiments, “a patient in need of therapy for hyperlipidemia or atherosclerosis” is a patient with an elevated blood level of glycerol. In such embodiments, “identifying” in step (a) above may be done by measuring the patient's level of blood glycerol, e.g., by forming a detectable complex of glycerol obtained from the blood of the patient and a reagent that binds to glycerol in order to determine the patient's level of blood glycerol, where an elevated level of blood glycerol identifies the patient as being in need of therapy for hyperlipidemia or atherosclerosis. In certain embodiments, the reagent is an antibody or antibody fragment that binds to glycerol. In certain embodiments, the antibody or antibody fragment that binds to glycerol is labeled (e.g., radioactively, antigenically, fluorescently, with peroxidase etc.) and measuring the patient's level of blood glycerol includes the step of detecting a physical transformation in the label (e.g., radioactive decay of the label) or in a substance acted upon by the label (oxidation of a substrate by a peroxidase label).

In certain embodiments, “identifying” in step (a) above includes transforming glycerol from a bodily sample from the patient into a derivative of glycerol, e.g., by phosphorylating glycerol in a reaction catalyzed by glycerol kinase to form glycerol-1-phosphate. In certain embodiments, “identifying” in step (a) above includes subjecting glycerol or a glycerol derivative from a bodily sample from the patient to chromatography where the glycerol is separated from the components of the blood with which it is normally found and interacts with the stationary phase used in the chromatographic process.

In certain embodiments, the patient's level of blood glycerol is determined to be elevated in comparison to a standard level of blood glycerol that has previously been determined to be a normal level. In other embodiments, the patient's level of blood glycerol is determined to be elevated in comparison to a level of blood glycerol measured in a person who is known not to be in need of therapy for hyperlipidemia or atherosclerosis. In other embodiments, the patient's level of blood glycerol is determined to be elevated in comparison to a level of blood glycerol measured in the patient at a time when the patient was known not to be in need of therapy for hyperlipidemia or atherosclerosis.

In certain embodiments, “a patient in need of therapy for hyperlipidemia or atherosclerosis” is a patient with an elevated blood level of free fatty acids. In such embodiments, “identifying” in step (a) above may be done by measuring the patient's level of blood free fatty acids, e.g., by forming a detectable complex of free fatty acids obtained from the blood of the patient and a reagent that binds to free fatty acids in order to determine the patient's level of blood free fatty acids, where an elevated level of blood free fatty acids identifies the patient as being in need of therapy for hyperlipidemia or atherosclerosis. In certain embodiments, the reagent is an antibody or antibody fragment that binds to free fatty acids. In certain embodiments, the antibody or antibody fragment that binds to free fatty acids is labeled (e.g., radioactively, antigenically, fluorescently, with peroxidase etc.) and measuring the patient's level of blood free fatty acids includes the step of detecting a physical transformation in the label (e.g., radioactive decay of the label) or in a substance acted upon by the label (oxidation of a substrate by a peroxidase label).

In certain embodiments, “identifying” in step (a) above includes transforming free fatty acids from a bodily sample from the patient into a derivative of free fatty acids, e.g., esters of the free fatty acids such as methyl esters. In certain embodiments, “identifying” in step (a) above includes subjecting free fatty acids or derivatives of free fatty acids from a bodily sample from the patient to chromatography where the free fatty acids are separated from the components of the blood with which they are normally found and interact with the stationary phase used in the chromatographic process.

In certain embodiments, the patient's level of blood free fatty acids is determined to be elevated in comparison to a standard level of blood free fatty acids that has previously been determined to be a normal level. In other embodiments, the patient's level of blood free fatty acids is determined to be elevated in comparison to a level of blood free fatty acids measured in a person who is known not to be in need of therapy for hyperlipidemia or atherosclerosis. In other embodiments, the patient's level of blood free fatty acids is determined to be elevated in comparison to a level of blood free fatty acids measured in the patient at a time when the patient was known not to be in need of therapy for hyperlipidemia or atherosclerosis.

The present invention encompasses a TPH1 inhibitor for use in the prevention or treatment of hyperlipidemia or atherosclerosis in a patient in need of such prevention or treatment. The present invention also encompasses a TPH1 inhibitor for use in lowering the blood levels of cholesterol, triglycerides, glycerol, or free fatty acids in a patient in need of such lowering of blood levels of cholesterol, triglycerides, glycerol, or free fatty acids. The patient is preferably a mammal, e.g., a human. The TPH1 inhibitor may be a therapeutic agent selected from therapeutic agents (1)-(189) disclosed herein or the therapeutic agents listed in Table 1.

The present invention encompasses the use a TPH1 inhibitor for the manufacture of a medicament for preventing or treating hyperlipidemia or atherosclerosis. In certain embodiments, the present invention encompasses the use of a therapeutic agent selected from therapeutic agents (1)-(189) disclosed herein or the therapeutic agents listed in Table 1 for the manufacture of a medicament for preventing or treating hyperlipidemia or atherosclerosis.

The present invention encompasses the use a TPH1 inhibitor for the manufacture of a medicament for lowering the blood levels of cholesterol, triglycerides, glycerol, or free fatty acids in a patient in need of such lowering. In certain embodiments, the present invention encompasses the use of a therapeutic agent selected from therapeutic agents (1)-(189) disclosed herein or the therapeutic agents listed in Table 1 for the manufacture of a medicament for lowering the blood levels of cholesterol, triglycerides, glycerol, or free fatty acids in a patient in need of such lowering.

### Pharmaceutical Compositions

Therapeutic agents such as the TPH1 inhibitors described herein may be formulated into pharmaceutical compositions. The therapeutic agents may be present in the pharmaceutical compositions in the form of salts of pharmaceutically acceptable acids or in the form of bases. The therapeutic agents may be present in amorphous form or in crystalline forms, including hydrates and solvates. Preferably, the pharmaceutical compositions comprise a therapeutically effective amount of a TPH1 inhibitor.

Pharmaceutically acceptable derivatives of any of the TPH1 inhibitors described herein are within the scope of the invention. A “pharmaceutically acceptable derivative” of a TPH1 inhibitor means any non-toxic derivative of a TPH1 inhibitor described herein that, upon administration to a patient, exhibits that same or similar biological activity with respect to reducing serum or plasma serotonin expression as the TPH1 inhibitor described herein.

Pharmaceutically acceptable salts of the therapeutic agents described herein for use in treating or preventing hyperlipidemia or atherosclerosis include those salts derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate salts. Other acids, such as oxalic, while not in themselves pharmaceutically acceptable, may be employed in the preparation of salts useful as intermediates in obtaining the therapeutic agents of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. The present invention also envisions the quaternization of any basic nitrogen-containing groups of the therapeutic agents disclosed herein. Water or oil-soluble or dispersible products may be obtained by such quaternization.

The therapeutic agents disclosed herein are also meant to include all stereochemical forms of the therapeutic agents (i.e., the R and S configurations for each asymmetric center). Therefore, the use of single enantiomers, racemic mixtures, and diastereomers of the therapeutic agents is within the scope of the invention. Also within the scope of the invention is the use of steric isomers and positional isomers of the therapeutic agents. The therapeutic agents of the present invention are also meant to include compounds which differ only in the presence of one or more isotopically enriched atoms. For example, therapeutic agents in which one or more hydrogens are replaced by deuterium or tritium, or the replacement of one or more carbons by ¹³C- or ¹⁴C-enriched carbon, are within the scope of this invention.

In a particular embodiment, the therapeutic agents of the present invention are administered in a pharmaceutical composition that includes a pharmaceutically acceptable carrier, adjuvant, or vehicle. The term “pharmaceutically acceptable carrier, adjuvant, or vehicle” refers to a non-toxic carrier, adjuvant, or vehicle that does not destroy or significantly diminish the pharmacological activity of the therapeutic agent with which it is formulated. Pharmaceutically acceptable carriers, adjuvants or vehicles that may be used in the pharmaceutical compositions of this invention encompass any of the standard pharmaceutically accepted solid carriers as well as liquid carriers such as a phosphate-buffered saline solution, water, as well as emulsions such as an oil/water emulsion or a triglyceride emulsion. An example of an acceptable triglyceride emulsion useful in the intravenous and intraperitoneal administration

of the compounds is the triglyceride emulsion commercially known as INTRALIPID®. Solid carriers may include excipients such as starch, milk, sugar, certain types of clay, stearic acid, talc, gums, glycols, or other known excipients. Carriers may also include flavor and color additives or other ingredients.

In the practice of the invention, the pharmaceutical compositions of the present invention are preferably administered orally. However, the pharmaceutical compositions may be administered parenterally, by inhalation spray, topically, rectally, nasally, buccally, vaginally or via an implanted reservoir. Preferably, the pharmaceutical compositions are administered orally, intraperitoneally or intravenously. Sterile injectable forms of the pharmaceutical compositions may be aqueous or oleaginous suspensions. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solutions. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, solid forms such as capsules and tablets. In the case of tablets for oral use, carriers commonly used include microcrystalline cellulose, lactose and corn starch. Lubricating agents, such as magnesium stearate, are also typically added. When aqueous suspensions are required for oral use, the active ingredient may be combined with emulsifying and suspending agents. If desired, certain sweetening, flavoring or coloring agents may also be added.

The pharmaceutical compositions of this invention may also be administered by nasal aerosol or inhalation. Such pharmaceutical compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Should topical administration be desired, it can be accomplished using any method commonly known to those skilled in the art and includes but is not limited to incorporation of the pharmaceutical composition into creams, ointments, or transdermal patches.

The TPH1 inhibitors of the present invention can be derivatized by the formation of a reversible linkage with one or more suitable groups to yield “pro-drugs,” i.e., chemical derivatives that, after absorption by the host, are converted into the parent compound. Liberation of the parent compound may be by chemical hydrolysis or enzymatic attack. A derivative or pro-drug can have enhanced permeability for the target organ. In the case of TPH1 inhibitors, the target organ is the duodenum where 95% of peripheral serotonin is made. The prodrug has an enhanced permeability according to the present invention if, after administration of the pro-drug or derivative thereof to a living organism, a higher amount of the compound reaches the target organ, resulting in a higher level of effective therapeutic agent, as compared to administration of the base compound without derivatization.

The amount of the therapeutic agents of the present invention that may be combined with the carrier materials to produce a pharmaceutical composition in a single dosage form will vary depending upon the patient being treated and the particular mode of administration. It should be understood that a specific dosage and treatment regimen for any particular patient will depend upon a variety of factors, including the activity of the specific therapeutic agent employed, the age, body weight, general health, sex, diet, time of administration, rate of excretion, drug combination, and the judgment of the treating physician, as well as the severity of the particular condition being treated. Despite their variety, accounting for these factors in order to select an appropriate dosage or treatment regimen is routinely done in the art and thus would require no more than routine experimentation.

The dosage of TPH1 inhibitor administered may also depend on whether the TPH1 inhibitor is being administered for the prevention or for the treatment of hyperlipidemia or atherosclerosis. For prevention, preferred dose ranges include from about 5 mg/kg/day to about 250 mg/kg/day; from about 5 mg/kg/day to about 100 mg/kg/day; or from about 5 mg/kg/day to about 30 mg/kg/day; with about 10 mg/kg/day being especially preferred.

For treatment, preferred dose ranges include from about 10 mg/kg/day to about 250 mg/kg/day; from about 10 mg/kg/day to about 50 mg/kg/day; or from about 10 mg/kg/day to about 30 mg/kg/day; with about 25 mg/kg/day being especially preferred.

Additional drugs which are normally administered to treat hyperlipidemia or atherosclerosis may also be present in the pharmaceutical compositions of this invention. Those additional drugs may be administered separately from the therapeutic agents that are used to lower serum or plasma serotonin levels, as part of a multiple dosage regimen. Alternatively, those additional drugs may be part of a single dosage form, mixed together with the therapeutic agents that are used to lower serum or plasma serotonin levels in a single pharmaceutical composition. If administered as part of a multiple dosage regime, the additional drugs and the therapeutic agents used to lower serum or plasma serotonin levels may be administered simultaneously, sequentially or within a selected specified period of time from one another. The amount of both the therapeutic agent that is used to lower serum or plasma serotonin levels and the additional drug (in those compositions which comprise an additional drug) that may be combined with the carrier materials to produce a single dosage form will vary depending upon the patient treated and the particular mode of administration as well as on the nature of the therapeutic agent that is used to lower serum or plasma serotonin levels and the nature of the additional drug.

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

### EXAMPLES

### Example 1

Mouse models of atherosclerotic plaque development, administration of therapeutic agent, and analysis of aortas

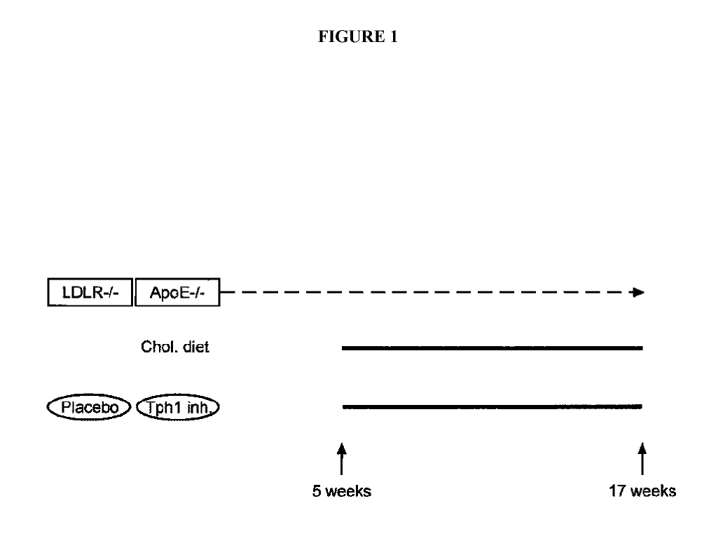

To study the effect of gut-derived serum or plasma serotonin on the development of atherosclerosis, two well-studied genetic mouse models of hypercholesterolemia and atherosclerosis were used, namely mice with deletions in both copies of the apoliprotein E gene (referred to as ApoE−/− mice) and mice carrying deletions of both copies of the low density lipoprotein receptor gene (LDLR−/− mice). These mice have elevated blood cholesterol levels due to a defect in hepatic clearance of cholesterol containing very low density lipoproteins and low density lipoproteins (Ishibashi et al., 1993, J. Clin. Invest. 92:883-893; Plump et al., 1992, Cell 71:343-353). To accelerate the process of atherosclerotic plaque development, the mice were placed on a high cholesterol rodent diet at 5 weeks of age and fed this diet for 12 weeks. During this period, the mice were orally administrated placebo (diluent) or LP-533401, an inhibitor of tryptophan hydroxylase 1 (TPH1), the key enzyme required for serum or plasma serotonin synthesis, at the dose of 200 mg per kg of body weight per day (FIG. 1).

After 12 weeks of high cholesterol diet and administration of therapeutic agent or placebo, the mice were sacrificed to determine the amount of atheromas in their aortas. The mice were given an overdose of anesthesia (ISOFLUORANE®) and blood was collected by heart puncture through the skin using an EDTA coated needle to prevent coagulation. Next, chests were opened and the heart was exposed to rinse out the blood from the vessels and clear the vessels using cold PBS. The whole aorta was then dissected and put into 4% paraformaldehyde in PBS. The aorta from the aortic arch up to the bifurcation was cut longitudinally and spread on silicon coated Petri dishes. The spread vessels were fixed over night in 4% paraformaldehyde and stained with Oil Red O (a lipid stain) to distinguish plaques from normal vessel wall. A quantitative computer-assisted image analysis (ImageJ) of the plaque staining areas was performed and correlated with the whole vessel wall areas.

### Example 2

Measurement of plasma cholesterol, triglyceride, glycerol, and free fatty acid levels

Cholesterol, triglycerides, glycerol, and free fatty acids were measured in plasma from 4 hours fasted mice using commercially available kits (Cholesterol E, Wako, Cat. No. 439-17501; Triglycerides determination kit, Sigma, Cat. No. TR0100-1KT (measures both triglycerides and glycerol), HR Series NEFA-HR, Wako, Cat. No. 999-34691) according to the manufacturer's instructions.

### Example 3

### Plasma Serotonin Determination

Serotonin content in the plasma was determined using Serotonin research ELISA (IBL, Cat. No. IB89540) according to the manufacturer's instructions.

### Example 4

### Adipose Tissue Explants—Isolation and Stimulation

Epigonadal fat pads were isolated from 8 week old wild type male mice, cut into 20 mg fragments, and placed in culture medium (DMEM low glucose, 0.1% bovine serum albumin, both from Invitrogen) in a cell incubator (37° C., 5% CO₂ atmosphere) for 2 h. After this time, the fat explants were stimulated for 2 h with different concentrations of serotonin. Concentrations of free fatty acids (FFA) and glycerol were measured in the medium. For gene expression analysis, tissue was homogenized in TRIZOL® reagent (Sigma) and RNA was isolated according to the manufacturer's instructions. The RNA was used as a template for cDNA synthesis (cDNA synthesis kit, Invitrogen). The relative gene expression was quantified using Real Time PCR.

### Example 5

### Inhibition of Gut-Derived Serotonin Synthesis Decreases Development of Atherosclerosis

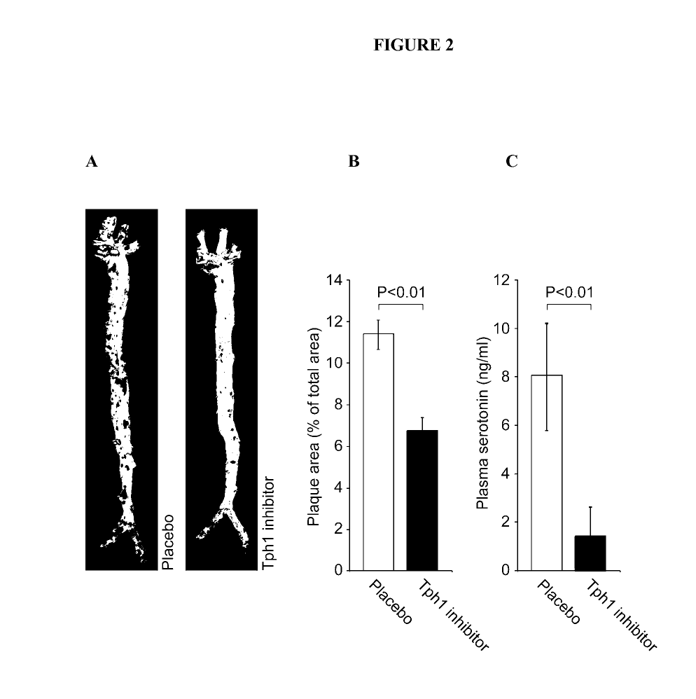

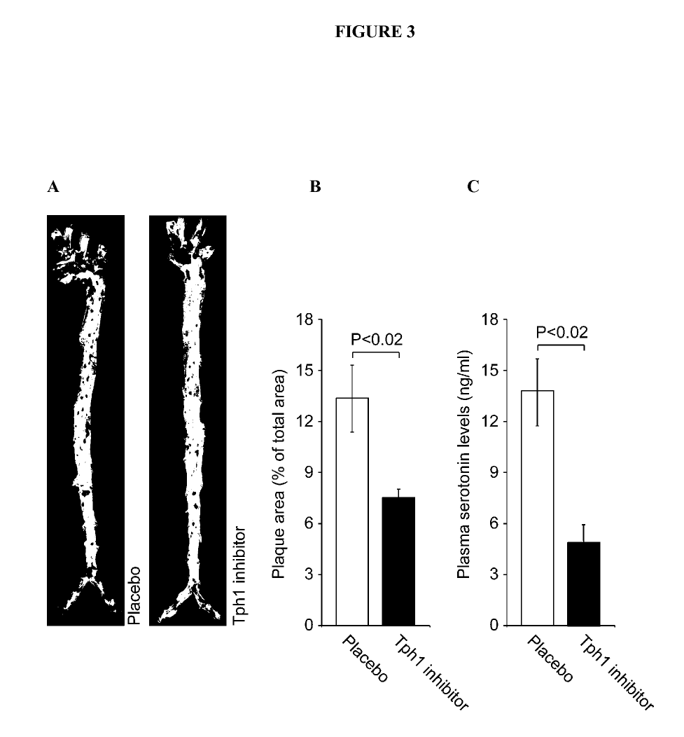

The amount of atherosclerotic plaques was determined in aortas from ApoE−/− mice treated with placebo or TPH1 inhibitor as described herein. Administration of TPH1 inhibitor resulted in a 44% reduction of the area occupied by atherosclerotic plaques (FIGS. 2A and 2B). Importantly, the dose of inhibitor used resulted in an 80% reduction of plasma serotonin levels (FIG. 2C). The same strategy was used to determine the amount of atherosclerotic plaques in LDLR−/− mice. Similarly to ApoE−/− mice, administration of TPH1 inhibitor resulted in reduction of serotonin levels in plasma (−60%, FIG. 3C) and significant (40%, FIGS. 3A and 3B) reduction in the amount of atherosclerotic plaques.

### Example 6

### Effect of Inhibition of Gut-Derived Serotonin Synthesis on Plasma Cholesterol and Triglyceride Levels

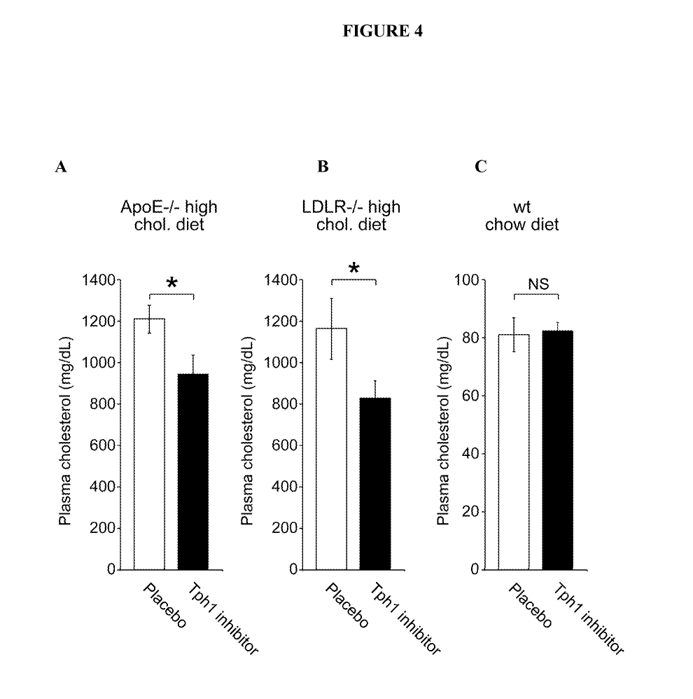

As the major factor predisposing for development of atherosclerosis is hyperlipidemia and especially high levels of plasma cholesterol, cholesterol and triglyceride levels were measured in plasma from ApoE−/− and LDLR−/− mice kept on a high cholesterol diet and treated with either placebo or TPH1 inhibitor. Strikingly, cholesterol levels in plasma of ApoE−/− mice treated with TPH1 inhibitor were 30% reduced in comparison to placebo treated control ApoE−/− mice (FIG. 4A). Similar reduction was observed in LDLR−/− mice dosed with an inhibitor of serotonin synthesis when compared with vehicle treated LDLR−/− control mice (FIG. 4B). Finally, cholesterol levels were measured in wild type mice treated with TPH1 inhibitor. Inhibition of serotonin synthesis did not alter cholesterol levels in wild type mice, suggesting that the serotonin inhibition ameliorates hypercholesterolemia but does not affect basal cholesterol levels (FIG. 4C).

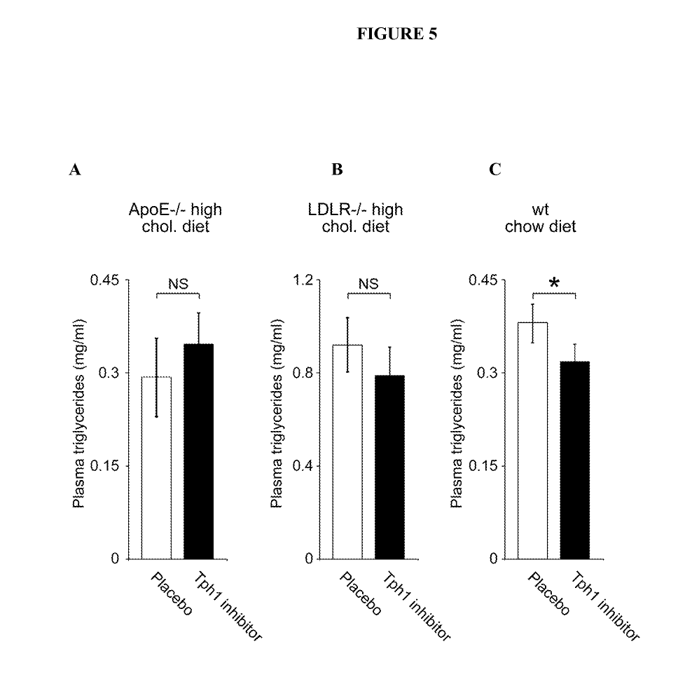

Inhibition of TPH1 did not alter triglyceride levels in ApoE−/− and LDLR−/− mice but attenuated triglyceride levels in wild type animals (FIG. 5A-C).

### Example 7

### Gut-Derived Serotonin Promotes Fat Store Mobilization from Adipose Tissue During Food Deprivation and TPH Inhibitors Reduce Plasma Levels of Glycerol and Free Fatty Acids

Mobilization of the energy deposits stored in adipose tissue under conditions of food deprivation is required for sustaining energy homeostasis. During times of undisturbed food availability, adipose tissue takes up and stores triglycerides in the form of lipid droplets. When nutritional factors are limited, adipose tissue supplies the energy to the rest of the organism in the form of highly energetic products of triglyceride breakdown—glycerol and free fatty acids (FFA)—in a process called lipolysis. Mobilization of FFA and glycerol is tightly controlled by lipolytic enzymes like hormone sensitive lipase (HSL) and adipose triglyceride lipase (ATGL), which are required for breakdown of triglycerides and therefore secretion of FFA and glycerol from adipose tissue (Zechner et al., 2005, Curr. Opin. Lipidol. 16:333-340).

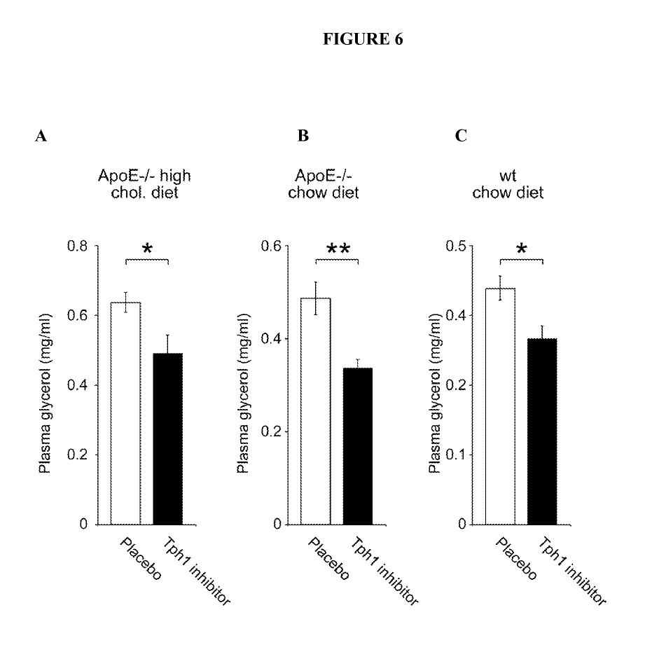

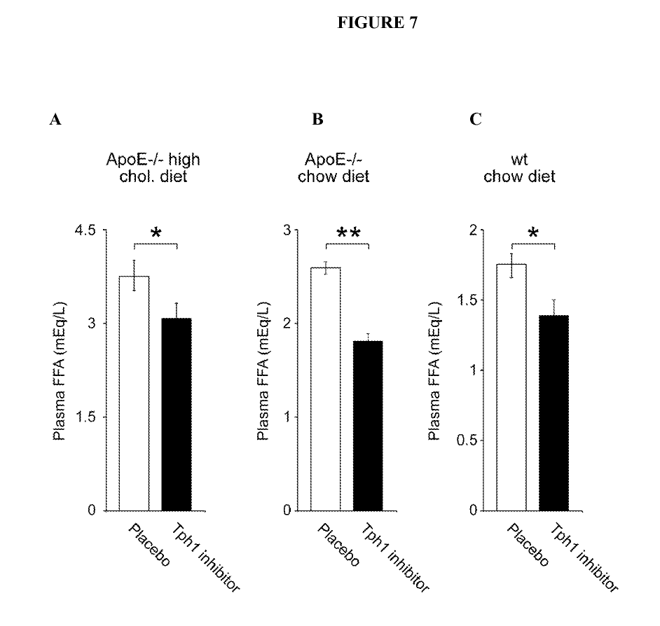

During the course of assessing lipid levels in mice treated with an inhibitor of serotonin synthesis, markedly reduced levels of glycerol were observed. Glycerol levels were reduced in ApoE−/− mice fed high cholesterol diet and normal rodent chow diet as well as in wild type mice treated with TPH1 inhibitor as compared to placebo treated control animals (FIG. 6A-C). Consistently, FFA levels were reduced in all the mouse strains mentioned above when treated with inhibitor of serotonin synthesis (FIG. 7A-C). These data indicate that gut-derived serotonin is a hormone required for efficient induction of lipolysis.

### Example 8

### Serotonin Promotes Lipolysis in Adipose Tissue

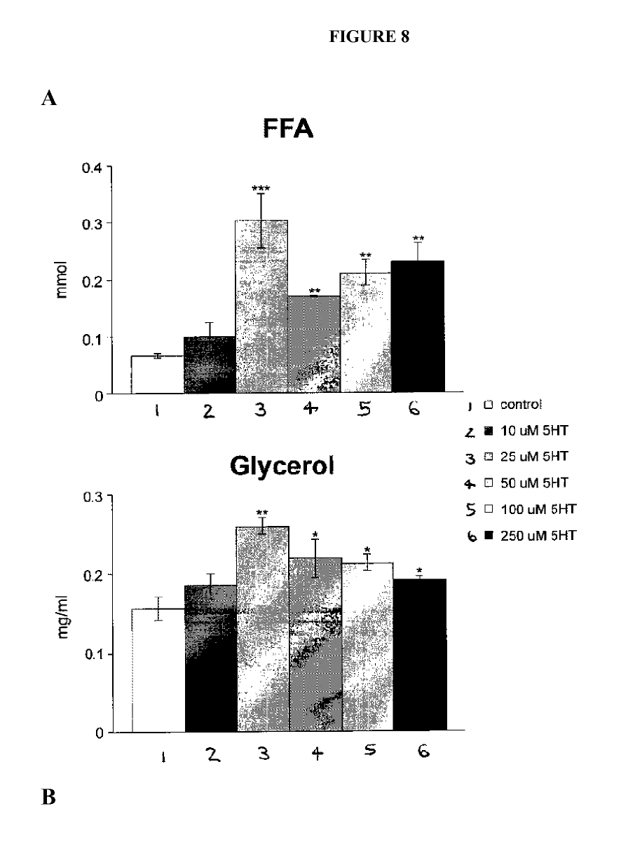

To test if serotonin stimulates triglyceride breakdown by acting directly on adipose tissue, white adipose tissue explants were prepared and stimulated ex vivo with different concentrations of serotonin. Serotonin stimulated secretion of FFA and glycerol from the adipose tissue at all tested concentrations. The highest stimulation was obtained by 25 μM serotonin (FIG. 8).

### Example 9

### Serotonin Promotes the Expression of Hormone Sensitive Lipase

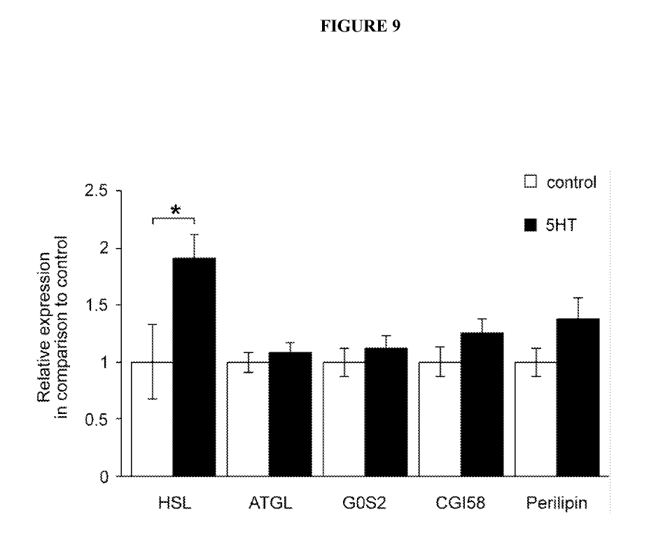

On the molecular level serotonin stimulation promotes the expression of hormone sensitive lipase (HSL) but does not affect another lipolytic enzyme, adipose triglyceride lipase (ATGL), and does not influence the expression levels of other components of the lipolytic machinery such as perilipin, GOS2 and CGI58 (FIG. 9).

### Example 10

### Serotonin Promotes Mobilization of Fat Stores

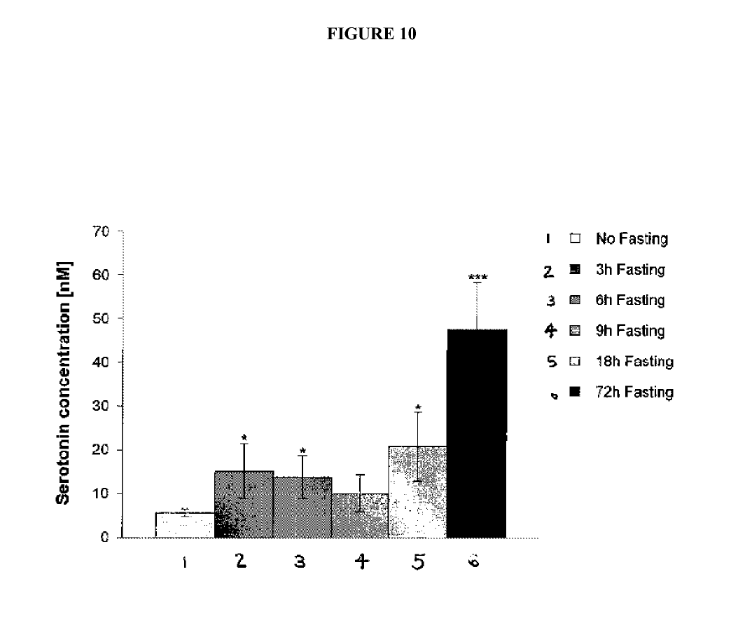

Mobilization of fat stores from adipose tissue is required during fasting. The data described herein raise the possibility that gut-derived serotonin is the hormone of the fasting stage of the organism. To test this hypothesis plasma serotonin levels in fasted mice were measured at different time points of food deprivation. Serotonin plasma levels were increased as early as 3 hours after food removal and remained elevated at all the time points tested, reaching the highest levels after 72 hours of fasting (FIG. 10).

What is claimed is:

1. A method of treating or preventing hyperlipidemia or atherosclerosis in a patient known or suspected to be in need of such treatment or prevention comprising administering to the patient known or suspected to be in need of such treatment or prevention a therapeutically effective amount of a tryptophan hydroxylase 1 (TPH1) inhibitor.

2. A method of lowering plasma cholesterol, lowering plasma triglycerides, lowering plasma glycerol, or lowering plasma free fatty acids in a patient known or suspected to be in need of such lowering of plasma cholesterol, lowering of plasma triglycerides, lowering of plasma glycerol, or lowering of plasma free fatty acids comprising administering to the patient a therapeutically effective amount of a TPH1 inhibitor.

3. The method of claim 1 or 2 wherein the TPH1 inhibitor is selected from the following or from pharmaceutically acceptable salts and/or solvates thereof: 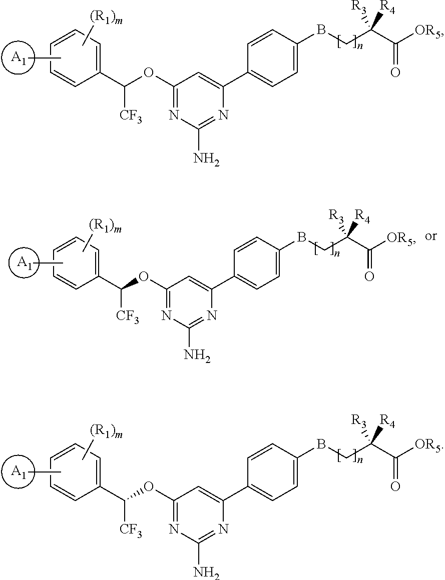
where A₁ is optionally substituted heterocycle or 3-fluorophenyl; B is O, N, or —CH₂—; each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is halogen, hydrogen, C(O)R_{A}, OR_{A}, NR_{B}R_{C}, S(O₂)R_{A}, or optionally substituted alkyl, alkyl-aryl or alkyl-heterocycle; either R₃ is NHR₆ and R₄ is hydrogen or, alternatively, R₃ and R₄ together form ═O; R₅ is hydrogen or optionally substituted alkyl, alkyl-aryl, alkyl-heterocycle, aryl, or heterocycle; R₆ is hydrogen, C(O)R_{A}, C(O)OR_{A}, or optionally substituted alkyl, alkyl-aryl, alkyl-heterocycle, aryl, or heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; m is 0-4; and n is 0 or I.

4. The method of claim 1 or 2 wherein the TPH1 inhibitor is

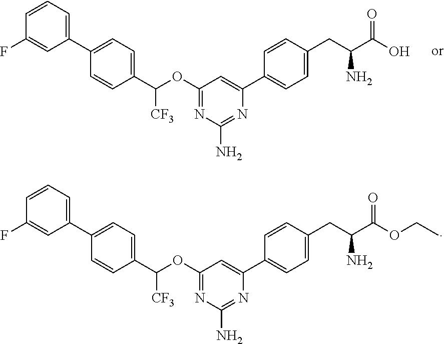 [SMILES: Nc1nc(OC(c2ccc(-c3cccc(F)c3)cc2)C(F)(F)F)cc(-c2ccc(C[C@H](N)C(=O)O)cc2)n1 ; "or" ; CCOC(=O)[C@@H](N)Cc1ccc(-c2cc(OC(c3ccc(-c4cccc(F)c4)cc3)C(F)(F)F)nc(N)n2)cc1]

5. The method of claim 1 or 2 wherein the TPH1 inhibitor does not cross the blood brain barrier or the TPH1 inhibitor does not significantly inhibit tryptophan hydroxylase 2 (TPH2).

6. The method of claim 1 or 2 wherein the patient's level of serum or plasma serotonin is measured prior to administering the TPH1 inhibitor.

7. The method of claim 1 or 2 wherein the patient's level of serum or plasma serotonin is measured after administering the TPH1 inhibitor.

8. The method of claim 1 or 2 wherein the patient is a mammal.

9. The method of claim 1 or 2 wherein the patient is a human.

10. The method of claim 1 or 2 wherein the TPH1 inhibitor is administered with another compound that is known to prevent or treat hyperlipidemia or atherosclerosis.

11. The method of claim 1 or 2 wherein the TPH1 inhibitor is administered with an inhibitor of hormone sensitive lipase.

12. A method for preventing or treating hyperlipidemia or atherosclerosis comprising:
(a) identifying a patient in need of therapy for hyperlipidemia or atherosclerosis;
(b) administering to the patient a therapeutically effective amount of a TPH1 inhibitor that decreases serum or plasma serotonin levels in order to prevent or treat hyperlipidemia or atherosclerosis in the patient identified in step (a).

13. A method for identifying a patient having hyperlipidemia or atherosclerosis or at risk of developing hyperlipidemia or atherosclerosis and treating the patient, comprising:
a) determining the level of serum or plasma serotonin in a biological sample taken from the patient and in a biological sample taken from a normal subject;
b) administering to the patient a therapeutically effective amount of a TPH1 inhibitor if the level of serum or plasma serotonin in the sample from the patient is elevated above the serum or plasma serotonin level in the sample from the normal subject;
whereby the patient's serum or plasma serotonin level is lowered and hyperlipidemia or atherosclerosis is thereby treated.
